(19) United States
(12) Patent Application Publication
(10) Pub. No.: US 2025/0283153 A1
(43) Pub. Date: Sep. 11, 2025

(54) MULTIPLEX METHODS OF DETECTING MOLECULES USING NANOPORES

(71) Applicants: Oxford Nanopore Technologies PLC, Oxford (GB); Imperial College Innovations Limited, London (GB)

(72) Inventors: Andrew John Heron, Oxford (GB); Richard Alexander Gutierrez, Oxford (GB); Joshua Edel, London (GB); Aleksandar Ivanov, London (GB); Caroline Koch, London (GB); Liang Xue, London (GB); Benedict Reilly-O'Donnell, London (GB)

(73) Assignees: Oxford Nanopore Technologies PLC, Oxford (GB); Imperial College Innovations Limited, London (GB)

(21) Appl. No.: 18/561,853

(22) PCT Filed: May 19, 2022
(86) PCT No.: PCT/GB2022/051268

§ 371 Date: Nov. 17, 2023

(30) Foreign Application Priority Data
May 19, 2021 (GB) 2107192.3

(51) Int. Cl. C12Q 1/6825; C12Q 1/6869

(52) U.S. Cl. CPC C12Q 1/6825; C12Q 1/6869

(57) ABSTRACT

A method for detecting multiple molecules in a sample, the method comprising: (a) contacting the sample with a carrier and a nanopore, wherein the carrier comprises a single-stranded leader, an identifier region and a molecule-binding region specific for a molecule to be detected, and wherein a motor protein is bound to the carrier such that it can control the movement of the identifier region within the nanopore; (b) taking one or more optical or electrical measurements as a carrier moves within the nanopore to characterise the identifier region and to determine whether or not the molecule is bound to the molecule-binding region.

### RELATED APPLICATIONS

This application is a national stage filing under 35 U.S.C. § 371 of international application number PCT/GB2022/051268, filed May 19, 2022, which claims the benefit of United Kingdom application number GB 2107192.3, filed May 19, 2021, each of which is herein incorporated by reference in its entirety.

### REFERENCE TO AN ELECTRONIC SEQUENCE LISTING

The contents of the electronic sequence listing (0036670147US00-SUBSEQ-CBD.txt; Size: 98,433 bytes; and Date of Creation: Jun. 6, 2024) is herein incorporated by reference in its entirety.

### FIELD

The present invention relates to multiplex methods of detecting molecules in a sample using nanopore technology. The invention also relates to carriers for binding and identifying molecules, and populations of such carriers, and kits and systems comprising such carriers.

### BACKGROUND

Biological sensors are a vital part of medical diagnostics and are part of a rapidly growing industry. Current state-of-the-art detection techniques used for biomarker (e.g. protein) sensing are usually coupled with a quantitative optical readout through ELISA assays, or a qualitative colour readout and are limited by low concentrations, that mask rare events. Antibody-based detection techniques are usually limited in scope to polypeptide targets and suffer from low sensitivity at low concentrations. Mass-spectrometry (MS)-based technologies generally require extensive sample preparation and may also suffer from low sensitivity at low concentrations, whilst targeted-MS techniques require large sample sizes if multiple targets are to be analysed.

Nanopore technology has previously been used to detect non-polynucleotide molecules. WO 2013/121201 describes a method for determining the presence or absence of one or more molecules using probes comprising aptamers and transmembrane pore technology, and exemplifies the detection of thrombin. DNA carriers have been used previously to enable selective, label-free detection of targets, yet are limited to single analytes and cannot be easily expanded (see, for example, Sze et al. “Nature comms 8.1 (2017): 1-10, and Cai et al. Nature comms 10.1 (2019): 1-9).

Furthermore, some populations of biomarkers, e.g. miRNA populations, have short lifetimes and are present at low concentrations, which means that they are currently clinically inaccessible.

Accordingly, there is a need for analytical methods that can achieve simultaneous detection of multiple soluble proteins, miRNAs and other molecules such as biomarkers in complex samples, such as biological fluids. Furthermore, there is also a need for such technologies to be able to detect very low concentrations of the target molecules. A technology that can achieve this holds the promise of far-reaching impact, for example in healthcare for diagnostics and monitoring disease progression. Such technologies could also find application in distinct fields, such as studying water samples for the presence of pollutants or other contaminants.

### SUMMARY

The disclosure relates to a method of utilizing nanopore technology, and the like, to detect proteins, miRNA and other biomarkers, as well as other types of molecule. The technology has the potential for highly multiplexed detection, directly in unprocessed samples, with high sensitivity and a rapid read-out.

Accordingly, provided herein is:
a method for detecting multiple molecules in a sample, the method comprising:
(a) contacting the sample with a carrier and a nanopore, wherein the carrier comprises a single-stranded leader, an identifier region and a molecule-binding region specific for a molecule to be detected, and wherein a motor protein is bound to the carrier such that it can control the movement of the identifier region within the nanopore;
(b) taking one or more optical or electrical measurements as a carrier moves within the nanopore to characterise the identifier region and to determine whether or not the molecule is bound to the molecule-binding region;
a carrier comprising a single-stranded leader, an identifier region and a molecule-binding region specific for a molecule to be detected, wherein a motor protein is bound to the carrier at a position between the single-stranded leader and the identifier region;
a population of carriers for multiple molecules, wherein each carrier comprises a single-stranded leader, an identifier region and a molecule-binding region specific for a molecule to be detected, wherein a motor protein is bound to the carrier at a position between the single-stranded leader and the identifier region, and different carriers in the population comprise different identifier regions and different molecule-binding regions;
a kit for detecting multiple molecules in a sample, comprising:
(i) a population of carriers, wherein each carrier comprises an identifier region and a molecule-binding region specific for a molecule to be detected, and different carriers in the population comprise different identifier regions and different molecule-binding regions;
(ii) an adaptor comprising a single-stranded leader; and
(ii) a motor protein; and
a system for detecting multiple molecules in a sample, comprising:
(i) a population of carriers, wherein each carrier comprises a single-stranded leader, an identifier region and a molecule-binding region specific for a molecule to be detected, wherein a motor protein is bound to the carrier at a position between the single-stranded leader and the identifier region, and different carriers in the population comprise different identifier regions and different molecule-binding regions; and
(iii) a nanopore.

### BRIEF DESCRIPTION OF THE FIGURES

FIG. 1. Schematic illustration of the barcode sequencing and the detection of biomarkers. Overall a custom designed strand mainly consisting of a barcode and a binding region (e.g. cDNA, aptamer, antibody) with targeted analyte bound is tethered to the membrane. The target-bound strand is detected by translocating through a biological nanopore (CsgG).

FIG. 2. (A) Schematic of an exemplary complete carrier including (i) a leader for facilitating threading into the nanopore, (ii) a tether with a cholesterol linker to enhance capture rate, (iii) a motor protein which provides squiggle signals for sequencing the barcode when coupling with the nanopore under an applied voltage, (iv) a polynucleotide identifier section (e.g. a barcode or multiple barcodes that may be repeated), (v) a spacer(s) that connects the barcode and (vi) a molecule-binding region, such as an aptamer/c-miRNA/antibody, which selectively targets, for example, miRNAs, proteins or neurotransmitters. (B) Schematic of an exemplary complete carrier including an adapter that consists of (i) a leader for facilitating threading into the nanopore, (ii) a tether with a cholesterol linker to enhance the capture rates, (iii) a motor protein which provides squiggle signals for sequencing the barcode when coupling with the nanopore under an applied voltage. The adapter is ligated to a DNA strand consisting of (i) the adapter ligation part which is hybridised to the complementary strand to with a single A overhang, (ii) a polynucleotide identifier section (a barcode or multiple barcodes that may be repeated), (iii) spacer(s) which connect the barcode to (iv) one or multiple molecule-binding regions, such as an aptamer/c-miRNA/antibody which selectively binds, for example, target miRNAs, proteins or neurotransmitters.

FIG. 3. Illustration of carriers and method for determining presence or absence of a molecule on a molecule-binding region. Enzymatic digestion is used to remove/digest any molecule-binding region that is not bound to the molecule for which it is specific. (a) A site for an nicking enzyme or endonuclease is included in the carrier such that it is hidden from the nicking enzyme or endonuclease when the molecule-binding region is bound to the molecule for which it is specific, but exposed when the molecule is not bound to the molecule-binding region. After contacting the sample with the carrier under conditions suitable for the molecule-binding region to the molecule for which it is specific, a nicking enzyme or endonuclease may be added such that a nick is introduced in any carriers in which the molecule-binding region is not bound to the molecule for which it is specific. After such digestion, the carriers that are bound to the target molecule can be distinguished from carriers that are bound to the target molecule based on the presence or absence of the current signal after the signal produced by the identifier region (barcode sequence). (b) After contacting the sample with the carrier under conditions suitable for the molecule-binding region to the molecule for which it is specific, an exoonuclease may be added such the molecule-binding region is digested. After such digestion, the carriers that are bound to the target molecule can be distinguished from carriers that are bound to the target molecule based on the presence or absence of the current signal after the signal produced by the identifier region (barcode sequence).

FIG. 4. Barcode sequencing and demultiplexing. (a) All sequences are basecalled with the basecalling algorithm (see slide 19-25) and aligned to the reference sequences, which are the barcode sequences. The max alignment score is used to classify the barcode. If the max alignment score and the second highest are too close together, the event won't be classified. Furthermore, the p-value is used to classify events and remove false positive classifications. From top to bottom, SEQ ID NOs: 144-154 are shown. (b) With this method an accuracy of 99.95% is achieved with 86% of all recorded events being used and classified.

FIG. 5. Sequencing, alignment and barcode classification. (a) Barcode sequencing has accuracy of >90%, with a chance of 0.0001% for false positives. (b) A confusion matrix showing very low preference for the wrong barcode classification.

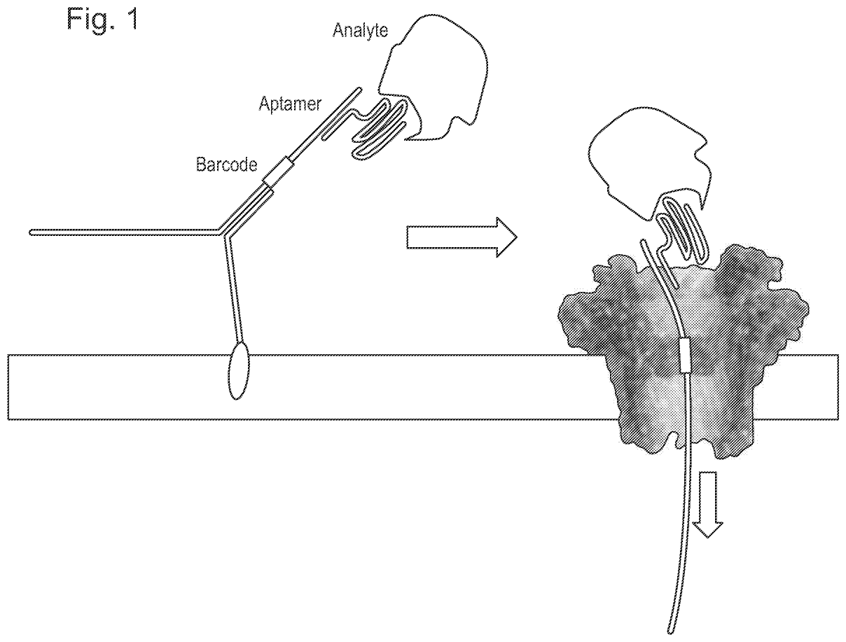

Barcode 1:
(SEQ ID NO: 9)
TGCTACTCTCCTCATAAGCAGTCCGGTGTATCGAT,
Barcode 2:
(SEQ ID NO: 10)
ATCGCTACGCCTTCGGCTCGTAATCATAGTCGAGT,
Barcode 3:
(SEQ ID NO: 11)
AGCTCAGAGCAGGTCACTCAAGATACGAGCTGCGT,
Barcode 4:
(SEQ ID NO: 12)
GTAAGTCTGCATCAGCGCGCGGCTGTGCGAGGATA,
Barcode 5:
(SEQ ID NO: 13)
CTACGACAGTACGCTAGCAAGGATAGACACTACGA,
Barcode 6:
(SEQ ID NO: 14)
TACTGAACACAAGTTCGTCGTCGAGCAATCACAAT,
Barcode 7:
(SEQ ID NO: 15)
AGTCTACCATTACTTGGATCGGATTAGCCTCACTC,
Barcode 8:
(SEQ ID NO: 16)
TGCACGAGTGCGTGTCAACCGTCCAGATGCTCGTG,
Barcode 9:
(SEQ ID NO: 17)
CTAGTGCGCAGTIGTCTCGGCGGAGTTGAGACTGA,
Barcode 10:
(SEQ ID NO: 18)
GATCATGGTAGTCTTCAAGATCGAGTATGTCTGTC.

FIG. 6. 10 barcoded carriers were discriminated in a complex mixture. At the same concentrations no bias was observed in barcoded carrier detection rates. The barcodes used were barcodes 1-10 above.

FIG. 7. Stalling analysis was used to determine whether a target has bound to the barcoded strand or not. (a) If the target analyte is not bound to the barcoded strand the current signal does not indicate stalling (in dwell time, current amplitude). (b) Bound analytes (here an example is given with a complementary miRNA) stall the carrier which results an unique current profile.

FIG. 8. Stalling of carrier (Barcode 6) without miRNA is significantly less (6.4%), than when miRNA is added (62.18%).

FIG. 9. Multiplexed detection of miRNAs with concentration dependence. 10 different barcodes enable the detection and discrimination of 10 different miRNAs. There is some observed heterogeneity in capture rate but dynamic range remained similar at 0-5 nM miRNA. The barcodes used were barcodes 1-10 above.

miRNA 1:
(SEQ ID NO: 19)
CAGCAGCACACUGUGGUUUGU,
miRNA 2:
(SEQ ID NO: 20)
AGAGCUUAGCUGAUUGGUGAAC,
miRNA 3:
(SEQ ID NO: 21)
UAGCUUAUCAGACUGAUGUUG,
miRNA 4:
(SEQ ID NO: 22)
ACCUGGCAUACAAUGUAGAUUU,
miRNA 5:
(SEQ ID NO: 23)
UGUAAACAUCCCCGACUGGAAG,
miRNA 6:
(SEQ ID NO: 24)
UGUAAACAUCCUACACUCUCAGC,
miRNA 7:
(SEQ ID NO: 25)
AGCUGGUAAAAUGGAACCAAAU,
miRNA 8:
(SEQ ID NO: 26)
GAGCUUUUGGCCCGGGUUAUAC,
miRNA 9:
(SEQ ID NO: 27)
AACAUUCAUUGCUGUCGGUGGGU,
miRNA 10:
(SEQ ID NO: 28)
UAGCACCAUCUGAAAUCGGUUA.

FIG. 10. Detection of binding between thrombin and a 15-mer thrombin binding aptamer. Upon binding with thrombin, the squiggle events showed much longer dwell time, with significant increase in stalling found upon binding with 400 nM thrombin in terms of current flipping, corresponding to the unwinding of G-quadruplex and aptamer-protein interactions. The first thrombin carrier provided in Example 1 was used in this experiment.

FIG. 11. Concentration dependence of the binding between thrombin and a 15-mer thrombin binding aptamer. The binding was verified by increasing thrombin concentration from 0 nM to 400 nM. As the thrombin concentration increased, an increase in stalling of the carrier was observed as more squiggle events with longer dwell time. The first thrombin carrier provided in Example 1 was used in this experiment.

FIG. 12. Detection of serotonin using the stem-loop aptamer. The barcode sequence associated with the serotonin aptamer provided in Example 1 was used. The structure of serotonin and the aptamer in the carrier are shown. An example current trace showing the signal produced as the barcode interacts with the pore and the signal produced as the aptamer interacts with the pore is provided. The average delay caused by unfolding of the aptamer increases with serotonin concentration as shown in the table and graph. From left to right, SEQ ID NOs: 155, 227, and 162 are shown.

FIG. 13. Detection of serotonin using the stem-loop aptamer. (A) Current traces in the absence of serotonin and in the presence of 40 mM serotonin. The dwell time increases in the presence of serotonin as shown in the current vs dwell time plots. (B) Shows the current vs dwell time plots for 0 mM, 2.5 mM, 5 mM, 10 mM, 20 mM and 40 mM serotonin. A concentration dependent increase in stalling events was observed.

FIG. 14. Detection of acetylcholine using a stem-loop aptamer. The barcode and aptamer sequences used are shown. Example measurements of the carrier without and with acetylcholine are provided. A correlation between concentration of acetylcholine and delay percentage was observed. From left to right, SEQ ID NOs: 48 and 175 are shown.

FIG. 15. Detection of molecules without motor protein. (A) Detection of increasing concentrations of thrombin using a thrombin-binding aptamer (TBA). Shaded regions indicate the signal of the TBA. Events with a signal at a lower nA indicate TBA-bound thrombin. SEQ ID NO: 142 is shown. (B) Detection of increasing serotonin concentrations using a serotonin-binding aptamer (SBA). Shaded regions relate to signals observed for SBA alone of SBA-bound serotonin. SEQ ID NO: 143 is shown. (C) Detection of barcodes in a multiplexed sample. Barcodes are distinguished based of the amplitude of the signal of the barcode region.

FIG. 16. Multiplexed screening and detection strategy example. A population of carriers may be used to generate a biological passport for screening and diagnostics.

FIG. 17. Sequences of carrier strands—miRNAs. From top to bottom, the sequences to the left of /iSpC3//iSpC3/ are SEQ ID NOs: 50-89. From top to bottom, the sequences to the right of /iSpC3//iSpC3/ are SEQ ID NOs: 176-215.

FIG. 18. Sequences of carrier strands—proteins and neurotransmitters. From top to bottom, the sequences to the left of /iSpC3//iSpC3/ are SEQ ID NOs: 90-98. From top to bottom, the sequences to the right of /iSpC3//iSpC3/ are SEQ ID NOs: 216-224.

FIG. 19. Confusion matrix of barcode classification.

FIG. 20. Detection of multiple miRNAs A. Increase in translocation time of barcoded sample (Barcode 13) with 10 nM miRNA, compared to control (0 nM). B. (TOP) Characteristic current trace of a barcode event with associated moving standard deviation plot. If moving standard deviation drops below a certain threshold (0.003), the event was classified as delayed. (BOTTOM) Characteristic current trace of a delayed event and its associated moving standard deviation plot. C. Single barcode (Barcode 38) titration curve (n=5). D. Multiplexed titration curves of 40 different barcodes with increasing (respective) miRNA concentrations in the same sample (n=5). E. Boxplot showing the delays detected for 10 nM miRNA added in a multiplexed experiment (n=5), overlayed with a scatter of an individual experiment for each barcode with 10 nM miRNA added (n=1, dots).

FIG. 21. Quantification of unknown miRNA concentrations in multiplexed experiment. (A) Titration curves for the 40 barcodes multiplexed experiment were plotted individually. Each curve was fitted with the Hill fit function. True concentration of added miRNA (dark grey) and the predicted concentration of added miRNA (light grey) determined based on the standard curves. The results show very high overlap between the predicted and actual concentration. (B) Residual analysis showing the difference between the predicted value minus the actual value (n=12).

FIG. 22. Detection of cTnI. A: cTnI aptamer sequence (SEQ ID NO: 49). B: Comparison of event times+/−30 ng/ml cTnI B Total event time. C: Event time to C3 peak. D: Event time from C3 peak to end. Concentration-delay relationship of cTnI, events are ‘delayed’ where t>95% ile of control events. E: Total event time. F: Event time to C3 peak. G: Event time from C3 peak to end.

### DETAILED DESCRIPTION

### Molecular Carriers and Multiplex Methods

The disclosure relates to methods of detecting multiple molecules in a sample. The methods comprise contacting the sample with a carrier, wherein the carrier comprises an identifier region associated with a molecule-binding region specific for a molecule to be detected. When the carrier moves within a detector, such as a pore, under the control of a motor protein, the identifier region is characterized and whether or not a molecule is bound to the molecule-binding region is determined. The method therefore identifies whether the molecule to be detected is present or absent from the characterization of the associated identifier region and the determination of whether or not a molecule is bound to the molecule-binding region. Multiple molecules within the sample can be detected. As explained in more detail below, the method allows for multiple molecules in a sample to be correctly detected and identified.

As discussed above, methods for identifying molecules in a sample are known in the art. One method that is known in the art involves a probe comprising an aptamer and a tail (see WO 2013/121201). Different probes with different aptamers and different tails are provided, and each tail may have a different effect on the current flowing through a pore, depending on whether or not an analyte is bound to the aptamer. In this way, the method may detect multiple analytes in a sample. The presence or absence of an analyte bound to an aptamer is detected by a stalling of the movement of the tail through the pore when the analyte is bound to the aptamer, when compared to the a probe without an analyte bound to the aptamer.

However, the methods of the prior art are limited by the diversity and the number of distinct tails of the probes that can be generated. Different analytes are distinguished from one another merely by varying the lengths of the probes, the presence or absence of double-stranded regions in the tails of the probes, and the different binding affinities of the different aptamers in each carrier. These limitations limit the number of different analytes in the sample that can be distinguished from one another.

The inventors have devised a way to distinguish between large numbers of different analytes and/or carriers within a sample. The inventors have devised a carrier comprising a motor protein located on the carrier such that it can control the movement of a molecule-specific identifier region within a pore. The controlled movement allows for an accurate characterisation of the identifier region, for example by sequencing. The differences between the identifier regions in carriers comprising a motor protein that are designed to detect different molecules can be much less when the movement of the carrier through the pore is controlled in this way. The carriers devised by the present inventors therefore allow for the label-free identification of large numbers of distinct identifier regions. A highly multiplexed method for identifying multiple analytes in a single sample can therefore be performed using the carriers. By including alternative identifier regions for carriers contacted with different samples, the methods of the invention also allow for the simultaneous measurement of multiples analytes from multiple samples.

The improved methods disclosed herein perform well at detecting very low levels of analytes in a sample (i.e. have a high sensitivity). The methods disclosed herein enable efficient detection and screening of, for example, rare protein and miRNA molecules using ultra-dilute samples (sub pico-molar levels) and can achieve single-molecule sensitivity in a high throughput manner. The method disclosed herein may, for example, be used to detect molecules, such as proteins or polynucleotides such as miRNA, present in a sample at concentrations as low as from about 1 pM to about 1 fM. Standard aptamer-based methods of detecting molecules require higher concentrations of analytes in order to produce a detectable signal. The methods disclosed herein enable detection at the single-molecule level, and can be used to determine the concentration of molecules in a sample. The specific binding of molecules to the molecule-binding regions of the carriers allows the effective concentration of the molecule to be increased, for example by localization to the membrane comprising a pore via a membrane anchors on the carrier. The carriers disclosed herein also provide the benefit of allowing multiplex detection of molecules in unprocessed samples, such as, for example, native clinical samples.

Accordingly, provided herein is a method for detecting multiple molecules in a method for detecting multiple molecules in a sample, the method comprising:
(a) contacting the sample with a carrier and a nanopore, wherein the carrier comprises a single-stranded leader, an identifier region and a molecule-binding region specific for a molecule to be detected, and wherein a motor protein is bound to the carrier such that it can control the movement of the identifier region within the nanopore;
(b) taking one or more optical or electrical measurements as a carrier moves within the nanopore to characterise the identifier region and to determine whether or not the molecule is bound to the molecule-binding region.

### Characterizing the Identifier Regions

The carrier comprises one or more identifier region(s). The methods require that the carrier interacts with a detector, e.g. moves within a pore, to characterize the identifier region. Suitable measurements that can be taken to characterize the identifier region are discussed below.

Preferably, the identifier region comprises, or is, a polynucleotide. Preferably, the polynucleotide sequence of the identifier region is determined. Any suitable technique may be used. The disclosure is particularly suited to single-molecule characterisation and the detection of low concentrations of molecule. Exemplary suitable sequencing techniques are discussed in more detail herein. For example, in some preferred embodiments, the sequencing technique is a nanopore sensing method. Nanopore sensing methods are described in detail here. However, the methods disclosed herein are not limited to nanopore sensing. Other single molecule sequencing technologies are amenable to the methods disclosed herein.

In nanopore strand sequencing, the identifier region moves within the pore. The signal recorded as the identifier region moves within the pore allows the sequence of the identifier region to be determined. Other characteristics of the identifier region, for example (i) the length of the polynucleotide, (ii) the identity of the polynucleotide, (iii) the secondary structure of the polynucleotide and (iv) whether or not the polynucleotide is modified, can alternatively or additionally be determined to characterise the identifier region.

The presence of the carrier molecule in the channel of a nanopore has an effect on the open-channel ion flow through the pore. This is the essence of “molecular sensing” of pore channels. Variation in the open-channel ion flow can be measured using suitable measurement techniques, e.g. by the change in electrical current (for example, WO 2000/28312 and D. Stoddart et al., Proc. Natl. Acad. Sci., 2010, 106, 7702-7 or WO 2009/077734). The degree of reduction in ion flow, as measured by the reduction in electrical current, is related to the size of the obstruction within, or in the vicinity of, the pore. Analogous information can be obtained using optical methods, for example as disclosed in Huang et al., Nature Nanotechnology 10, 986-991(2015). Binding of a molecule of interest (e.g. the target polynucleotide) in or near the pore therefore provides a detectable and measurable event, thereby forming the basis of a “biological sensor”.

As a nucleic acid molecule, or an individual base, moves within a pore (e.g. as it passes through the channel of a nanopore), the size differential between the bases causes a directly correlated reduction in the ion flow through the channel. The variation in ion flow may be recorded. Suitable electrical measurement techniques for recording ion flow variations are described in, for example, WO 2000/28312 and D. Stoddart et al., Proc. Natl. Acad. Sci., 2010, 106, pp 7702-7 (single channel recording equipment); and, for example, in WO 2009/077734 (multi-channel recording techniques). Through suitable calibration, the characteristic reduction in ion flow can be used to identify the particular nucleotide and associated base traversing the channel in real-time. In typical nanopore nucleic acid sequencing, the open-channel ion flow is reduced as the individual nucleotides of the nucleic sequence of interest sequentially pass through the channel of the nanopore due to the partial blockage of the channel by the nucleotide. It is this reduction in ion flow that is measured using the suitable recording techniques described above. The reduction in ion flow may be calibrated to the reduction in measured ion flow for known nucleotides through the channel resulting in a means for determining which nucleotide is passing through the channel, and therefore, when done sequentially, a way of determining the nucleotide sequence of the nucleic acid passing through the nanopore. For the accurate determination of individual nucleotides, the reduction in ion flow through the channel is typically required to be directly correlated to the size of the individual nucleotide passing through the constriction (or “reading head”). It will be appreciated that sequencing may be performed upon an intact nucleic acid polymer that is ‘threaded’ through the pore via the action of an associated motor protein, such as a polymerase or helicase, for example. Suitable motor proteins are described in more detail herein.

### Determining Molecule Binding

The methods determine whether or not a molecule is bound to the molecule-binding region of the carrier. In some embodiments, the method is used to detect the presence or absence of the molecules specifically bound to the carrier. The presence of absence of molecules specifically bound to the carrier is indicative of the presence of absence of the molecules in the sample(s).

The method may also be used to determine the concentration of the molecules, such as the relative-concentration of molecule-bound-carrier to free-carrier, or the absolute concentration of the molecules in the sample. The relative-concentration and absolute concentration may be calculated in any suitable way from the measurements obtained. Examples of methods of determining the relative-concentration and absolute concentration are provided in the Examples and the Figures.

The interaction of the molecule-binding region of the carrier with a detector is used to determine if a molecule is bound to the molecule-binding region of the carrier.

When using nanopore detection, the molecule-binding region effects the current flowing through the pore depending on whether or not the molecule-binding region is specifically bound to a molecule. The molecule-binding region affects the current flowing through the pore in one way when the molecule is not bound and affects the current flowing through the pore in a different way when the molecule is bound. This is important because it allows the presence or absence of a molecule specifically bound to the molecule-binding region and hence the presence or absence of a molecule in a sample to be determined using the method.

The signal produced by the molecule-specific identifier region (i.e. an identifier region present only in a carrier which carrier also comprises a molecule-binding region specific for a given molecule of interest) in the carrier is used to identify the molecule that is bound to, or not bound to, the carrier and hence to identify the molecule that is present, or absent, in the sample.

The effects of the molecule-binding region on the current flowing through the pore depending on whether or not the molecule is bound to a molecule-binding region can be measured based on the time it takes for the carrier, or the molecule-binding region of the carrier, to move within the pore. For example, when the molecule-binding region is an aptamer, the secondary and tertiary structure of the aptamer may detectably slow or temporarily stall the progression of the carrier within the pore. When the aptamer is bound to its cognate molecule, the carrier may have to overcome a higher energy barrier to progress within the pore and the speed of the progression of the aptamer through the pore may be detectably slower (such as an extended interaction) than when the aptamer is not bound to a molecule.

In some embodiments, the molecule-binding region may be a polynucleotide complementary to a target molecule, such as an miRNA. In such embodiments, when the molecule-binding region is not bound to the molecule, the carrier may progress within the pore at a “normal” speed. When the molecule-binding region is bound to its target molecule, such as an miRNA, the now double-stranded section of the molecule-binding region may affect the progression of the carrier within the pore such that the progressions is detectably slowed or stalled. In this way, the presence or absence of a molecule specifically bound to the molecule-binding region the carrier may be determined.

In some embodiments, the molecule-binding region may be an antibody, antibody-fragment, nanobody or affibody. Whilst the presence of such molecule-binding regions may prevent movements of the whole carrier through the pore, the presence or absence of the molecule may be determined. Without wishing to be bound by theory, such molecule-binding regions of the carrier act as a “leaky plug” to the pore. Nanopore measurements are extremely sensitive to small changes in the system, being able to discriminate between individual nucleotide bases, and so can detect differences in the “leakiness” of the plug dependent on whether or not a molecule is bound to the molecule-binding region.

Control experiments may be carried out to determine the effect the molecule-binding regions have on the current flowing through the pore and/or the progression of the carrier when the carrier is specifically bound to a the molecule compared to when the molecule is not bound. Results from carrying out the method described herein on a test sample can then be compared with those derived from such control experiments in order to determine whether a particular molecule is present or absent in the test sample. This is described in more detail in WO 2013/121201.

Alternative methods may be used to determine if a molecule is bound to the molecule-binding region or not. For example, digestion-based methods may be used, that rely on differences in the digestion of the molecule-binding region when a molecule is bound compared to when a molecule is not. Generally, a molecule bound to the molecule-binding region of the carrier may protect the molecule-binding region from digestion.

For instance, when the molecule-binding region is a polynucleotide, a site-specific endonuclease, such as a restriction enzyme or a single-stranded nicking enzyme, may be used. When the molecule is not bound, the polynucleotide molecule-binding region is digested and the absence thereof is detected as the carrier moves within the pore. When the molecule is bound, the polynucleotide molecule-binding region is not digested, or simply “nicked”, leading to a slowing or stalling of the progression of the carrier within the pore, as described above, and optionally the characterisation of the molecule-binding region.

Digestion methods may also be applied to instances wherein the molecule-binding region is a polynucleotide, such as an antibody, antibody-fragment, nanobody or affibody. Binding of a molecule to the molecule-binding region may lead to the protection of a protease target site and thus the molecule-binding region is not digested. In the absence of the molecule, the molecule-binding region may be digested. The difference in signal may be used to determine the presence or absence of the molecule on the carrier.

In some embodiments, a spacer as described herein may be positioned on the leader-sequence side of the molecule-binding region of the carrier. The spacer causes the progression of the carrier within the pore to slow or stall, thus providing an indicator of the position of the molecule-binding region in the measured signal. Furthermore, the presence of a spacer also allows slowing or stalling of the progression of the carrier within the pore to be exaggerated when a molecule is bound, thus providing a clearer signal to determine the presence or absence of a molecule on the carrier.

The methods also allow for the concentration of the molecules to be determined, as described in more detail in the Examples. In some embodiments, the relative concentration of molecule-bound-carrier compared to free-carrier in the sample is determined. This may be useful for the determination of relative changes in the levels of molecule between samples. In some embodiments, the absolute concentration of the molecule within a sample may be determined. This can be performed using a standard curve as a reference, as exemplified in the Examples.

The method is preferably a multiplex method allowing detection of multiple molecules simultaneously. For example, the method may be for detecting 2 or more, such as 5 or more, 10 or more, 50 or more, 100 or more, for example from 200 to 500, 500 or more, for example from 600 to 1000, or at least 1000, for example from 1000 to 10000, different molecules.

### Motor Proteins

As those skilled in the art will appreciate, any suitable motor protein can be used in the methods and products provided herein. A motor protein may be any protein that is capable of binding to a polynucleotide and controlling its movement with respect to a detector, such as a nanopore, e.g. through the pore. In some embodiments, more than one motor protein is bound to the carrier.

In some embodiments, a motor protein is or is derived from a polynucleotide handling enzyme. A polynucleotide handling enzyme is a polypeptide that is capable of interacting with and modifying at least one property of a polynucleotide. The enzyme may modify the polynucleotide by orienting it or moving it to a specific position.

In some embodiments, the motor protein is derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31.

Typically, the motor protein is a helicase, a polymerase, an exonuclease, a topoisomerase, or a variant thereof.

The motor protein is typically stalled on the carrier when the carrier is in solution. In some embodiments, the motor protein on carrier is modified to prevent the motor protein disengaging from the carrier (other than by passing off the end of the spacer). The motor protein can be adapted in any suitable way. For example, the motor protein can be loaded on the carrier and then modified in order to prevent it from disengaging from the spacer. Alternatively, the motor protein can be modified to prevent it from disengaging from the carrier before it is loaded onto the carrier. Modification of a motor protein in order to prevent it from disengaging from a carrier can be achieved using methods known in the art, such as those discussed in WO 2014/013260, which is hereby incorporated by reference in its entirety, and with particular reference to passages describing the modification of motor proteins such as helicases in order to prevent them from disengaging with polynucleotide strands.

For example, the motor protein may have a polynucleotide-unbinding opening; e.g. a cavity, cleft or void through which a polynucleotide strand may pass when the motor protein disengages from the strand. In some embodiments, the polynucleotide-unbinding opening is the opening through which a spacer may pass when the motor protein disengages from the spacer. In some embodiments, the polynucleotide-unbinding opening for a given motor protein can be determined by reference to its structure, e.g. by reference to its X-ray crystal structure. The X-ray crystal structure may be obtained in the presence and/or the absence of a polynucleotide substrate. In some embodiments, the location of a polynucleotide-unbinding opening in a given motor protein may be deduced or confirmed by molecular modelling using standard packages known in the art. In some embodiments, the polynucleotide-unbinding opening may be transiently produced by movement of one or more parts e.g. one or more domains of the motor protein.

The motor protein may be modified by closing the polynucleotide-unbinding opening. Closing the polynucleotide-unbinding opening may therefore prevent the motor protein from disengaging from the spacer. For example, the motor protein may be modified by covalently closing the polynucleotide-unbinding opening. In some embodiments, a preferred motor protein for addressing in this way is a helicase. In one embodiment, the motor protein is an exonuclease. Suitable enzymes include, but are not limited to, exonuclease I from E. coli (SEQ ID NO: 1), exonuclease III enzyme from E. coli (SEQ ID NO: 2), RecJ from T. thermophilus (SEQ ID NO: 3) and bacteriophage lambda exonuclease (SEQ ID NO: 4), TatD exonuclease and variants thereof. Three subunits comprising the sequence shown in SEQ ID NO: 3 or a variant thereof interact to form a trimer exonuclease.

In one embodiment, the motor protein is a polymerase. The polymerase may be PyroPhage® 3173 DNA Polymerase (which is commercially available from Lucigen® Corporation), SD Polymerase (commercially available from Bioron®), Klenow from NEB or variants thereof. In one embodiment, the enzyme is Phi29 DNA polymerase (SEQ ID NO: 5) or a variant thereof. Modified versions of Phi29 polymerase that may be used in the disclosure are disclosed in U.S. Pat. No. 5,576,204.

In one embodiment the motor protein is a topoisomerase. In one embodiment, the topoisomerase is a member of any of the Moiety Classification (EC) groups 5.99.1.2 and 5.99.1.3. The topoisomerase may be a reverse transcriptase, which are enzymes capable of catalysing the formation of cDNA from a RNA template. They are commercially available from, for instance, New England Biolabs® and Invitrogen®.

In one embodiment, the motor protein is a helicase. Any suitable helicase can be used in accordance with the methods provided herein. For example, the or each motor protein used in accordance with the present disclosure may be independently selected from a Hel308 helicase, a RecD helicase, a TraI helicase, a TrwC helicase, an XPD helicase, and a Dda helicase, or a variant thereof. Monomeric helicases may comprise several domains attached together. For instance, TraI helicases and TraI subgroup helicases may contain two RecD helicase domains, a relaxase domain and a C-terminal domain. The domains typically form a monomeric helicase that is capable of functioning without forming oligomers. Particular examples of suitable helicases include Hel308, NS3, Dda, UvrD, Rep, PcrA, Pif1 and TraI. These helicases typically work on single stranded DNA. Examples of helicases that can move along both strands of a double stranded DNA include FtfK and hexameric enzyme complexes, or multi-subunit complexes such as RecBCD. Hel308 helicases are described in publications such as WO 2013/057495, the entire contents of which are incorporated by reference. RecD helicases are described in publications such as WO 2013/098562, the entire contents of which are incorporated by reference. XPD helicases are described in publications such as WO 2013/098561, the entire contents of which are incorporated by reference. Dda helicases are described in publications such as WO 2015/055981 and WO 2016/055777, the entire contents of each of which are incorporated by reference.

In one embodiment the helicase comprises the sequence shown in SEQ ID NO: 6 (Trwc Cba) or a variant thereof, the sequence shown in SEQ ID NO: 7 (Hel308 Mbu) or a variant thereof or the sequence shown in SEQ ID NO: 8 (Dda) or a variant thereof. Variants may differ from the native sequences in any of the ways discussed herein. An example variant of SEQ ID NO: 8 comprises E94C/A360C. A further example variant of SEQ ID NO: 8 comprises E94C/A360C and then (ΔM1)G1G2 (i.e. deletion of M1 and then addition of G1 and G2).

In some embodiments a motor protein (e.g. a helicase) can control the movement of polynucleotides in at least two active modes of operation (when the motor protein is provided with all the necessary components to facilitate movement, e.g. fuel and cofactors such as ATP and Mg2+ discussed herein) and one inactive mode of operation (when the motor protein is not provided with the necessary components to facilitate movement).

When provided with all the necessary components to facilitate movement (i.e. in the active modes), the motor protein (e.g. helicase) moves along the polynucleotide in a 5′ to 3′ or a 3′ to 5′ direction (depending on the motor protein). In embodiments in which the motor protein is used to control the movement of a polynucleotide strand with respect to a nanopore, the motor protein can be used to either move the polynucleotide away from (e.g. out of) the pore (e.g. against an applied field) or the polynucleotide towards (e.g. into) the pore (e.g. with an applied field). For example, when the end of the polynucleotide towards which the motor protein moves is captured by a pore, the motor protein works against the direction of the field resulting from the applied potential and pulls the threaded polynucleotide out of the pore (e.g. into the cis chamber). However, when the end away from which the motor protein moves is captured in the pore, the motor protein works with the direction of the field resulting from the applied potential and pushes the threaded polynucleotide into the pore (e.g. into the trans chamber).

When the motor protein (e.g. helicase) is not provided with the necessary components to facilitate movement (i.e. in the inactive mode) it can bind to the polynucleotide and act as a brake slowing the movement of the polynucleotide when it is moved with respect to a nanopore, e.g. by being pulled into the pore by a field resulting from an applied potential. In the inactive mode, it does not matter which end of the polynucleotide is captured, it is the applied field which determines the movement of the polynucleotide with respect to the pore, and the motor protein acts as a brake. When in the inactive mode, the movement control of the polynucleotide by the motor protein can be described in a number of ways including ratcheting, sliding and braking.

In the active mode, motor proteins typically consume fuel molecules. Fuel is typically free nucleotides or free nucleotide analogues. The free nucleotides may be one or more of, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP). The free nucleotides are usually selected from AMP, TMP, GMP, CMP, UMP, dAMP, dTMP, dGMP or dCMP. The free nucleotides are typically adenosine triphosphate (ATP).

A cofactor for a motor protein is a factor that allows the motor protein to function. The cofactor is preferably a divalent metal cation. The divalent metal cation is preferably Mg²⁺, Mn²⁺, Ca²⁺ or Co²⁺. The cofactor is most preferably Mg²⁺.

In the methods described herein, the motor protein is bound to the carrier such that it can control the movement of the identifier region within a detector, such as a transmembrane pore.

The movement of the carrier within the detector may be controlled by any suitable means. In some embodiments, the movement of the construct is driven by a physical or chemical force (potential). In some embodiments the physical force is provided by an electrical (e.g. voltage) potential or a temperature gradient, etc.

In some embodiments, the detector is a nanopore and the construct moves with respect to the nanopore as an electrical potential is applied across the nanopore. Polynucleotides are negatively charged, and so applying a voltage potential across a nanopore will cause the polynucleotides to move with respect to the nanopore under the influence of the applied voltage potential. For example, if a positive voltage potential is applied to the trans side of the nanopore relative to the cis side of the nanopore, then this will induce a negatively charged analyte to move from the cis side of the nanopore to the trans side of the nanopore. Similarly, if a positive voltage potential is applied to the trans side of the nanopore relative to the cis side of the nanopore then this will impede the movement of a negatively charged analyte from the trans side of the nanopore to the cis side of the nanopore. The opposite will occur if a negative voltage potential is applied to the trans side of the nanopore relative to the cis side of the nanopore. Apparatuses and methods of applying appropriate voltages are described in more detail herein. In some embodiments the chemical force is provided by a concentration (e.g. pH) gradient.

### Sample

The sample may be any suitable sample. The sample may be a biological sample. Any of the methods described herein may be carried out in vitro on a sample obtained from or extracted from any organism or microorganism. The organism or microorganism is typically archaean, prokaryotic or eukaryotic and typically belongs to one of the five kingdoms: plantae, animalia, fungi, monera and protista. In some embodiments, the methods of various aspects described herein may be carried out in vitro on a sample obtained from or extracted from any virus.

The sample is preferably a fluid sample. The sample may be a complex biofluid. The sample typically comprises a body fluid. The body fluid may be obtained from a human or animal. The human or animal may have, be suspected of having or be at risk of a disease. The sample may be urine, lymph, saliva, mucus, seminal fluid, cerebrospinal fluid or amniotic fluid, whole blood, plasma or serum. Typically, the sample is human in origin, but alternatively it may be from another mammal such as from commercially farmed animals such as horses, cattle, sheep or pigs or may alternatively be pets such as cats or dogs.

Alternatively a sample of plant origin is typically obtained from a commercial crop, such as a cereal, legume, fruit or vegetable, for example wheat, barley, oats, canola, maize, soya, rice, bananas, apples, tomatoes, potatoes, grapes, tobacco, beans, lentils, sugar cane, cocoa, cotton, tea or coffee.

The sample may be a non-biological sample. The non-biological sample is preferably a fluid sample. Examples of non-biological samples include surgical fluids, water such as drinking water, sea water or river water, and reagents for laboratory tests.

The sample may be processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

In some embodiments, the sample may comprise genomic DNA. The genomic DNA may be fragmented or any of the methods described herein may further comprise fragmenting the genomic DNA. The DNA may be fragmented by any suitable method. For example, methods of fragmenting DNA are known in the art. Such methods may use a transposase, such as a MuA transposase, or a commercially available G-tube.

The method disclosed herein can be used to detect one or more molecule(s) from one or more sample(s), and to determine which sample the molecule is detected in, using a single assay. This can be achieved when a carrier comprising both an identifier region associated with a particular molecule binding region and an identifier region associated with a particular sample is used. The one or more sample(s), may be 2 or more, such as at least 3, 4, 5, 10, 20, 50 or 100 samples. The samples may be, for example, be taken from different patients, different types of tissue within a patient, or at different time points. The different time points may be separated by seconds, minutes, days, months or years. The samples may include one or more control sample(s).

The sample may be interrogated with no or minimal sample preparation, or the sample may be processed, for example to remove impurities or concentrate the type of molecule to be detected prior to use in the method. The ability to use an unprocessed or minimally processed sample leads to a rapid turnover from sample collection to analysis.

### Molecules

The carriers described herein comprise molecule-binding regions specific for a molecule to be detected. The methods disclosed herein are for detecting multiple molecules. The term “molecule” as provided herein may be used interchangeably with the term “analyte”.

The molecule may be any molecule that can be specifically bound by a molecule-binding region. For instance, the molecule may be metal ions, inorganic salts, polymers, amino acids, peptides, polypeptides, proteins, nucleotides, oligonucleotides, polynucleotides, dyes, bleaches, pharmaceuticals, diagnostic agents, recreational drugs, explosives and/or environmental pollutants. The molecules may be biomarkers. The method may comprise detecting two or more molecules of the same type, such as two or more proteins, two or more nucleotides or two or more pharmaceuticals. The method may comprise detecting two or more molecules of different types, such as one or more proteins, one or more nucleotides and one or more pharmaceuticals.

The molecules may be secreted from cells. Alternatively, the molecules may be present inside cells such that the molecules must be extracted from the cells before the method can be carried out.

In one embodiment, the molecules are selected from amino acids, peptides, polypeptides, proteins, nucleotides, oligonucleotides and/or polynucleotides.

In one embodiment, the molecules are selected from amino acids, peptides, polypeptides and/or proteins. The amino acids, peptides, polypeptides or proteins can be naturally-occurring or non-naturally-occurring. The polypeptides or proteins can include within them synthetic or modified amino acids. A number of different types of modification to amino acids are known in the art. Suitable amino acids and modifications thereof are discussed below with reference to the transmembrane pore. For the purposes of the disclosure, it is to be understood that the molecules can be modified by any method available in the art.

The proteins can be enzymes, antibodies, hormones, biomarkers, growth factors or growth regulatory proteins, such as cytokines. The cytokines may be selected from interleukins, such as IL-1, IL-1, IL-2, IL-4, IL-5, IL-6, IL-10, IL-12 and IL-13, interferons, such as IFN-γ, and other cytokines such as TNF-α. The proteins may be bacterial proteins, fungal proteins, virus proteins or parasite-derived proteins.

In one embodiment, the molecules are selected from nucleotides, oligonucleotides and/or polynucleotides. A nucleotide typically contains a nucleobase, a sugar and at least one phosphate group. The nucleobase is typically heterocyclic. Nucleobases include, but are not limited to, purines and pyrimidines and more specifically adenine, guanine, thymine, uracil and cytosine. The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, TMP, GMP, UMP, dAMP, dTMP, dGMP or dCMP. The nucleotides may be abasic (i.e. lack a nucleobase). The nucleotides may contain additional modifications. In particular, suitable modified nucleotides include, but are not limited to, 2′amino pyrimidines (such as 2′-amino cytidine and 2′-amino uridine), 2′-hydroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

Oligonucleotides are short nucleotide polymers which typically have 50 or fewer nucleotides, such 40 or fewer, 30 or fewer, 20 or fewer, 10 or fewer or 5 or fewer nucleotides. The oligonucleotides may comprise any of the nucleotides discussed above, including the abasic and modified nucleotides.

The polynucleotides may be single stranded or double stranded. At least a portion of the polynucleotide may be double stranded. The polynucleotides can be nucleic acids, such as deoxyribonucleic acid (DNA) or ribonucleic acid (RNA). The polynucleotides can comprise one strand of RNA hybridized to one strand of DNA. The polynucleotides may be any synthetic nucleic acid known in the art, such as peptide nucleic acid (PNA), glycerol nucleic acid (GNA), threose nucleic acid (TNA), locked nucleic acid (LNA) or other synthetic polymers with nucleotide side chains. The polynucleotides may comprise any of the nucleotides discussed above, including the modified nucleotides.

The polynucleotides can be any length. For example, the polynucleotides can be at least 10, at least 50, at least 100, at least 150, at least 200, at least 250, at least 300, at least 400 or at least 500 nucleotides or nucleotide pairs in length. The polynucleotides can be 1000 or more nucleotides or nucleotide pairs, 5000 or more nucleotides or nucleotide pairs in length or 100000 or more nucleotides or nucleotide pairs in length.

In one embodiment, the molecules are microRNAs (miRNAs). MiRNAs are single-stranded RNA polynucleotide molecules that play a role in post-transcriptional regulation of gene expression.

The molecules may be associated with a particular phenotype or with a particular type of cell. For instance, the molecules may be indicative of a bacterial cell. The molecules may be indicative of a virus, a fungus or a parasite. The molecules may be a specific panel of recreational drugs (such as the SAMHSA 5 panel test), of explosives or of environmental pollutants.

In one embodiment, the molecules are biomarkers that can be used to diagnose or prognose a disease or condition. The biomarkers may be any of the molecules mentioned above, such as proteins or polynucleotides. Suitable panels of biomarkers are known in the art, for example as described in Edwards, A. V. G. et al. (2008) Mol. Cell. Proteomics 7, p 1824-1837; Jacquet, S. et al. (2009), Mol. Cell. Proteomics 8, p 2687-2699; Anderson N. L. et al (2010) Clin. Chem. 56, 177-185. The disease or condition is preferably cancer, coronary heart disease, cardiovascular disease or sepsis.

In one embodiment, the molecules are neurotransmitters. Neurotransmitters are molecules that transmit signals between cells across a synapse. Examples of neurotransmitters include acetylcholine, dopamine, epinephrine, norepinephrine, nucleotides such as ATP, amino acids such as glutamate, aspartate and 6-aminobutyric acid, and enkephalins.

### Leader Sequence

The carriers of the disclosure comprise a single-stranded leader sequence. A leader sequence typically comprises a polymer such as a polynucleotide, for instance DNA or RNA, a modified polynucleotide (such as abasic DNA), PNA, LNA, polyethylene glycol (PEG) or a polypeptide. In some embodiments, the leader sequence comprises a single strand of DNA, such as a poly-dT section. The leader sequence can be any length, but is typically from 10 to 150 nucleotides in length, such as from 20 to 120, 30 to 100, 40 to 80 or 50 to 70 nucleotides in length.

### Identifier Region

The carriers of the disclosure comprise an identifier region. The carriers may comprise more than one identifier region, such as 2 or more, 3, 4, 5 or more, such as for example about 10 identifier regions. In some embodiments, the different identifier regions on a carrier may be associated with different molecule-binding regions, so that when an identifier region passes within the detector, the identity of the associated molecule-binding region(s) may be determined. Accordingly, a carrier may comprise a series of identifier regions and molecule-binding regions. The carrier is arranged such that the movement of the identifier region within the detector is controlled by a motor protein bound to the carrier.

In some embodiments, the presence of more than one identifier region on a carrier may be used to distinguish carriers from different samples. In some embodiments, a carrier comprising more than one identifier region may be used in a method for detecting multiple molecules in multiple samples, and so a carrier may comprise, for example, one identifier region unique to the sample and one identifier region unique to the molecule to which the carrier binds. The identifier region of the carrier is positioned such that when the carrier contacts a transmembrane pore, the motor protein bound to the carrier controls the movement of the identifier region within the transmembrane pore.

The purpose of the identifier region is to act as a unique signal of the identity of the molecule(s) that is bound to the carrier. A further identifier region may act as a unique signal of the source of the carrier, for example to identify the sample with which the carrier has been contacted. In some embodiments, the identifier region is a polynucleotide or comprises a polynucleotide sequence. The nucleotides may be any of those discussed below. The identifier polynucleotide may be from 2 to 300 nucleotides in length, such as 2 to 200, 2 to 100, 2 to 75, 2 to 50, 2 to 40, 2 to 30, 2 to 25, 4 to 100, 4 to 75, 4 to 50, 4 to 40, 4 to 30, 4 to 25, 4 to 20, 4 to 15, 4 to 10, 6 to 100, 6 to 75, 6 to 50, 6 to 40, 6 to 30, 6 to 25, 6 to 20, 6 to 15, 6 to 10, 8 to 100, 8 to 75, 8 to 50, 8 to 40, 8 to 30, 8 to 25, 8 to 20 or 8 to 15 nucleotides in length.

In some embodiments, the molecule-binding region or a part thereof is the identifier region. For example, the identifier region may overlap the molecule-binding region. In such embodiments, the molecule-binding region and the identifier region are preferably polynucleotides. When the molecule-binding region is a polynucleotide, such as an aptamer or a polynucleotide that hybridises to a target polynucleotide, the polynucleotide sequence of the molecule-binding region is unique to the bound molecule and thus acts to identify the molecule. In some embodiments, the identifier region and molecule-binding region do not overlap.

In some embodiments, the identifier region comprises a barcode sequence. Polynucleotide barcodes are well-known in the art (Kozarewa, I. et al, (2011), Methods Mol. Biol. 733, p 279-298). A barcode is a specific sequence of polynucleotide that affects the current flowing through the pore in a specific and known manner. The barcode sequence is typically 2 or more nucleotides in length, such as 4 or more, 8 or more, or 12 or more nucleotides in length. In some embodiments, the barcode sequence is 2 to 50 nucleotides in length, such as 2 to 45, 2 to 40, 2 to 35, 2 to 30, 2 to 25, 4 to 50, 4 to 45, 4 to 40, 4 to 35, 4 to 30, 4 to 25, 4 to 20, 4 to 15, 4 to 10, 6 to 50, 6 to 45, 6 to 40, 6 to 35, 6 to 30, 6 to 25, 6 to 20, 6 to 15, 6 to 10, 8 to 50, 8 to 45, 8 to 40, 8 to 35, 8 to 30, 8 to 25, 8 to 20, or 8 to 15 nucleotides in length. In some embodiments, the barcode sequence is 10 to 50, 10 to 45, 10 to 40, 10 to 35, 10 to 30, 10 to 25, or 10 to 20 nucleotides in length. In some embodiments, the barcode sequence is 15 to 50, 15 to 45, 15 to 40, 15 to 35, 15 to 30, or 15 to 25 nucleotides in length. In some embodiments, the barcode sequence is 20 to 50, 20 to 45, 20 to 40, 20 to 35, or 20 to 30 nucleotides in length. In some embodiments, the barcode sequence is 25 to 50, 25 to 45, 25 to 40, or 25 to 35 nucleotides in length. In some embodiments, the barcode sequence is about 10, 15, 20, 25, 30, 35, 40, 45 or 50 nucleotides in length.

The greater the length of the barcode, the greater the number of unique combinations that can be used. To increase the accuracy of barcode sequencing, the barcode may be repeated in the carrier to enable the barcode to be proof-read. Hence the identifier region may comprise 2 or more, such as 3 to 10, for example 3 or more, 4 or more, 5 or more, or 6 or more copies of the barcode.

Barcoding allows for highlight multiplexed detection. For example, a 4 base barcode will generate (4⁴=256) unique configurations which would allow for up to 256 protein or miRNA targets. Number of bases can be increased generating for example 4⁸, 4¹² unique combinations. For example, an 8 base sequence can be used to generate 65,536 unique barcodes. This is a huge advance in sensing and diagnostics in general where typically only one or a handful of molecules, such as up to about 5 or about 10 molecules, can be selectively probed at any one time.

In some embodiments, the identifier region may comprise a spacer or a series of spacers, as described herein. The series of spacers may comprise 2 or more, for example 3 or more, 4 or more, 5 or more, or 6 or more, 7 or more, 8 or more, 9 or more or 10 or more spacers. The series of spacers may comprise 20 or more, 50 or more, or 100 or more spacers. The series of spacers may comprise 2 to 1000 spacers, such as 2 to 100, 2 to 50, 2 to 20, or 2 to 10 spacers. The spacers in the series of spacers may be the same or different. As the identifier region moves within the pore, the characteristic signal of the spacer may be measured. The type and number of spacers in different carrier molecules may be distinguished based on the signals measured. The spacer or spacers may be any of the spacers described herein, for example, iSp9 and iSp18 spacers.

### Molecule-Binding Region

The carriers of the disclosure comprise a molecule-binding region specific for a molecule to be detected. In some embodiments, the carrier may comprise more than one molecule-binding region. A carrier may comprise one or more molecule-binding regions of the same type, and/or may comprise two or more different molecule-binding regions. The two or more molecule-binding regions may be 3 or more, such as 4, 5, 6 or more, such as for example about 10 molecule-binding regions. The different molecule-binding regions in the carrier typically bind specifically to different molecules. This allows the detection of multiple molecules (analytes) using a single carrier.

In the carrier an identifier region may be associated with each molecule-binding region. Typically the identifier region will be positioned such that it passes through the detector, such as a pore, prior to its associated molecule binding region interacting with the detector.

In the carrier, one identifier region may be associated with one or more molecule binding regions, such as 2 or more, 3 or more, for example from 4 to 10, molecule binding regions. In this situation, the 2 or more molecule binding regions typically bind to the same molecule. The 2 or more molecule binding regions may be specific for different molecules. The 2 or more molecule binding regions may be separated by a spacer or a series of spacers, such as those defined herein. The spacer may separate the molecule binding region from an associated identifier region.

Any molecule-binding region may be used, provided that it binds specifically to a molecule of interest such that when the carrier is contacted with the pore, the presence or absence of the molecule bound to the molecule-binding region can be determined. For example, a molecule-binding region may be: an aptamer; a complementary DNA sequence; a peptide or protein, such as an antibody, antibody fragment, nanobody or affibody; a click chemistry reactive group; biotin or streptavidin; or the like.

In one embodiment, the molecule-binding region is an aptamer. Aptamers are small molecules that bind to one or more molecules. Suitable aptamers and methods of producing aptamers are known in the art and are described, for example, in provided in WO 2013/121201, which is incorporated herein by reference. Aptamers can be produced using SELEX (Stoltenburg, R. et al., (2007), Biomolecular Engineering 24, p 381-403; Tuerk, C. et al., Science 249, p 505-510; Bock, L. C. et al., (1992), Nature 355, p 564-566) or NON-SELEX (Berezovski, M. et al. (2006), Journal of the American Chemical Society 128, p 1410-1411). The aptamer may be a peptide aptamer or an oligonucleotide aptamer. In one embodiment, the aptamer is a peptide aptamer. The peptide aptamer may comprise any amino acids. The amino acids may be any of those discussed below. In one embodiment, the aptamer is an oligonucleotide aptamer. The oligonucleotide aptamer may comprise any nucleotides. The nucleotides may be any of those discussed above. The aptamer can be any length. The aptamer is typically at least 15 amino acids or nucleotides in length, such as from about 15 to about 50, from about 20 to about 40 or from about 25 to about 30 amino acids or nucleotides in length.

In one embodiment, the molecule-binding region is a polynucleotide. In one embodiment, the polynucleotide is an aptamer. In one embodiment, the polynucleotide comprises a sequence complementary to a polynucleotide molecule to be detected (a target polynucleotide). A target polynucleotide can be any length. For example, the target polynucleotide can be at least 10, at least 50, at least 100, at least 150, at least 200, at least 250, at least 300, at least 400 or at least 500 nucleotides or nucleotide pairs in length. A target polynucleotide may be an oligonucleotide. Oligonucleotides are short nucleotide polymers which typically have 50 or fewer nucleotides, such 40 or fewer, 30 or fewer, 20 or fewer, 10 or fewer or 5 or fewer nucleotides. The molecule-binding region is preferably complementary to an miRNA. An miRNA is a short non-coding RNA with a role in post-transcriptional gene regulation, and is usually 21 to 23 nucleotides in length, but may be from 18 to 30 nucleotides in length, such as from 20 to 25 nucleotides in length.

Where the molecule to be detected is a polynucleotide, the molecule binding region may comprise a sequence that is 90% or more, such as at least 97%, 98% or 99% identical to the complement of the target polynucleotide. In such a molecule binding region, one or more, for example 2, 3, 4 or 5, nucleotides in the complement may be replaced with a non-canonical nucleotide that can base pair with the corresponding nucleotide in the target polypeptide. Preferably the molecule binding region comprises the complement of the target polynucleotide.

In one embodiment, the molecule binding region is an antibody, antibody fragment, nanobody or affibody. The term “antibody” as used herein may relate to whole antibodies (comprising two heavy chains and two light chains) as well as antigen-binding fragments thereof. Antibodies may include, but are not limited to, polyclonal, monoclonal, chimeric, dAb (domain antibody), single chain, Fab, Fab′ and F(ab′)2 fragments, and scFvs. Nanobodies are single-domain antibodies, such as V_{H}H fragments or V_{N}AR fragments. Affibodies are antibody mimetics comprising a three helix scaffold domain with amino acid substitutions on two of the three helices allowing for a large diversity in amino acid sequence and potential antigen binding. Affibodies are discussed in Frejd, Fredrik Y., and Kyu-Tae Kim. (Experimental & molecular medicine 49.3 (2017): e306-e306) and Löfblom, John, et al. (FEBS letters 584.12 (2010): 2670-2680). Suitable antibodies, antibody fragments, nanobodies and affibodies are known in the art or can be prepared by standard methods.

Methods of attaching a polypeptide are well known in the art. For example, site-specific C-terminal, N-terminal or internal loop labelling of proteins using sortase-mediated reactions may be used, as described in Guimaraes et al. Nature protocols 8.9 (2013): 1787, Theile et al. Nature protocols 8.9 (2013): 1800, and Koussa et al. Methods 67.2 (2014): 134-141, all of which are herein incorporated by reference. The skilled person can utilise suitable techniques to incorporate a protein, such as an antibody, into the carrier.

A molecule-binding region specific for a molecule to be detected, is able to bind to its intended target molecule (the molecule it is intended to detect) with greater affinity than it binds to an unrelated molecule. The unrelated molecule may be an unrelated control protein, such as bovine serum albumin, when the molecule to be detected is a protein. The unrelated molecule may be a scrambled control polynucleotide (e.g. a random polynucleotide sequence with the same numbers and types of nucleotides as the intended target molecule) when the molecule to be detected is a polynucleotide. The molecule to be detected preferably binds to the molecule-binding region with an affinity that is at least 10, at least 50, at least 100, at least 500, or at least 1000 times greater than the control. Affinity may be determined by methods known in the art. For example, affinity may be determined by ELISA assay, biolayer interferometry, surface plasmon resonance, kinetic methods or equilibrium/solution methods. The skilled person will recognize which molecules specifically bind a molecule-binding region.

Some cross-reactivity may occur, for example, with an miRNA polynucleotide with a similar sequence to the intended target miRNA molecule, or, with proteins sharing closely related domains. Preferably, the molecule binding region binds to its target molecule with greater affinity, for example, an affinity that is at least 10, at least 50, at least 100, at least 500, or at least 1000 times greater, than to a related molecule, such as a related polynucleotide, e.g. an miRNA polynucleotide with a similar sequence, or a related protein, such as a homologue.

### Spacers

In some embodiments of the methods provided herein, the carrier comprises a spacer. The spacer is preferably positioned between the bound motor protein and the molecule-binding region. The movement of the carrier within the pore is stalled (or, in other words, slowed or delayed) when the motor protein interacts with the spacer. The spacer is more preferably positioned immediately adjacent to the molecule-binding region. When the carrier moves within a detector, the movement of the motor protein is stalled by the spacer prior to the molecule-binding region interacting with the pore. When the motor protein is stalled at the spacer an exaggerated optical or electrical signal may be produced when a molecule is bound to the molecule-binding regions, as compared to a similar carrier without a spacer. Where the identifier region is separate from the molecule-binding region, the spacer is preferably located in the carrier between the identifier region and the molecule binding region.

As the carrier moves through the detector, e.g. as the carrier moves with respect to a nanopore, a distinctive electrical or optical signal is produced when the motor protein encounters the spacer. For example, a spacer positioned between the bound motor protein and the molecule-binding region may act as a distinctive signal to allow the signal produced when the molecule-binding region interacts with the detector to be clearly identified, e.g. located in the signal/trace/squiggle produced as the carrier moves within a nanopore. The spacer can thus be used as a marker to locate the signal produced as the molecule-binding region moves within the detector, facilitating the determination of the presence or absence of a molecule specifically bound to the molecule-binding region.

A spacer may provide an energy barrier which impedes movement of a motor protein. For example, a spacer may stall a motor protein by reducing the traction of the motor protein on the polynucleotide. This may be achieved for instance by using an abasic spacer i.e. a spacer in which the bases are removed from one or more nucleotides in the carrier.

A spacer may physically block movement of a motor protein, for instance by introducing a bulky chemical group to physically impede the movement of the motor protein. The spacer may be a double-stranded region of a polynucleotide.

The spacer may comprise a linear molecule, such as a polymer. Typically, the linear spacer has a different structure from the target polynucleotide. For instance, if the target polynucleotide is DNA, the or each spacer typically does not comprise DNA. In particular, if the target polynucleotide is deoxyribonucleic acid (DNA) or ribonucleic acid (RNA), the or each spacer preferably comprises peptide nucleic acid (PNA), glycerol nucleic acid (GNA), threose nucleic acid (TNA), locked nucleic acid (LNA) or a synthetic polymer with nucleotide side chains. In some embodiments, a spacer may comprise one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methylcytidines, one or more 5-hydroxymethylcytidines, one or more 2′-O-Methyl RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(O)NHCH₂—C₆H₃NO₂—CH(CH₃)OPO₃] groups, one or more hexandiol groups, one or more spacer 9 (iSp9) [(OCH₂CH₂)₃OPO₃] groups, or one or more spacer 18 (iSp18) [(OCH₂CH₂)₆OPO₃] groups; or one or more thiol connections. A spacer may comprise any combination of these groups. Many of these groups are commercially available from IDT® (Integrated DNA Technologies®). For example, C3, iSp9 and iSp18 spacers are all available from IDT®. A spacer may comprise any number of the above groups as spacer units.

In some embodiments, a spacer may comprise one or more chemical groups which cause a motor protein to stall. In some embodiments, suitable chemical groups are one or more pendant chemical groups. The one or more chemical groups may be attached to one or more nucleobases in the carrier. The one or more chemical groups may be attached to the backbone of the carrier. Any number of appropriate chemical groups may be present, such as 2, 3, 4, 5, 6, 7, 8, 9, 10, 11, 12 or more. Suitable groups include, but are not limited to, fluorophores, streptavidin and/or biotin, cholesterol, methylene blue, dinitrophenols (DNPs), digoxigenin and/or anti-digoxigenin and dibenzylcyclooctyne groups. In some embodiments, a spacer may comprise a polymer. In some embodiments the spacer may comprise a polymer which is a polypeptide or a polyethylene glycol (PEG).

In some embodiments, a spacer may comprise one or more abasic nucleotides (i.e. nucleotides lacking a nucleobase), such as 2, 3, 4, 5, 6, 7, 8, 9, 10, 11, 12 or more abasic nucleotides. The nucleobase can be replaced by —H (idSp) or —OH in the abasic nucleotide. Abasic spacers can be inserted into target polynucleotides by removing the nucleobases from one or more adjacent nucleotides. For instance, polynucleotides may be modified to include 3-methyladenine, 7-methylguanine, 1,N6-ethenoadenine inosine or hypoxanthine and the nucleobases may be removed from these nucleotides using Human Alkyladenine DNA Glycosylase (hAAG). Alternatively, polynucleotides may be modified to include uracil and the nucleobases removed with Uracil-DNA Glycosylase (UDG). In one embodiment, the one or more spacers do not comprise any abasic nucleotides.

One or more spacers may be present elsewhere in the carrier. The spacer may comprise any suitable number of spacers. For example, the carrier may comprise two or more, 3 or more or 5 or more spacers, such as from one to about 20 spacers, e.g. from 1 to about 10 spacers.

### Stall Region

In some embodiments, the carrier comprises a stall region. The stall region of the carrier provides a position for the motor protein to localise on the carrier when the carrier is in solution (i.e. before being contacted with, and moving within, the pore). The stall region is typically present between the leader and the identifier region. This enables the leader to interact with a detector, such as to thread into a pore. It also positions the motor protein on the carrier such that it is poised to control the movement of the identifier region through the detector, e.g. pore, upon interaction of the carrier with the detector.

In some embodiments, the stall region is a spacer, as described herein and in WO 2020/234612. The carrier may further comprise a blocking moiety, which prevents the motor protein from moving off the spacer.

A blocking moiety is typically a moiety which prevents the movement of the motor protein in the direction opposite to that in which the motor protein naturally processes a polynucleotide. For example, if the motor protein naturally processes a polynucleotide strand in the 5′ to 3′ direction, then a suitable blocking moiety may be a moiety which prevents the motor protein from moving in the 3′ to 5′ direction. Similarly, if the motor protein naturally processes a polynucleotide strand in the 3′ to 5′ direction, then a suitable blocking moiety may be a moiety which prevents the motor protein from moving in the 5′ to 3′ direction.

The blocking moiety is typically bound to carrier so as to prevent the movement of the motor protein off the spacer. Preventing the motor protein from moving off the spacer can be achieved by providing a steric block to physically prevent the movement of the motor protein. Preventing the movement of the motor protein from off the spacer can be achieved by using a chemical blocking moiety over or past which the motor protein cannot move. In some embodiments, the blocking moiety comprises one or more of the spacer groups discussed herein. In other embodiments, the blocking moiety may comprise a polynucleotide strand.

The carrier may also comprise a loading site connected to the stall region or spacer. A loading site is a site for loading the motor protein onto the polynucleotide adapter. Suitable loading sites are described in more detail in WO 2020/234612.

Methods of loading a motor protein onto a polynucleotide, stalling the motor protein on the polynucleotide in solution, suitable spacers and suitable blocking moieties are described in more detail in WO 2020/234612, incorporated herein by reference, and in WO 2014/135838, incorporated herein by reference.

### Anchor

In some embodiments, the carrier comprises a membrane anchor or a transmembrane pore anchor attached to the carrier. The anchor may be covalently or non-covalently attached to the carrier. For example, the anchor may be attached to an oligonucleotide hybridised to a polynucleotide region of the carrier. The polynucleotide region of the carrier to which the anchor-oligonucleotide is hybridised is distinct from the molecule binding region in the sense that it does not prevent specific binding of a molecule to the molecule binding region.

In some embodiments, the anchor aids in characterisation of a target polynucleotide in accordance with the methods disclosed herein. For example, in methods which comprise contacting the carrier with a transmembrane pore, a membrane anchor or transmembrane pore anchor may promote localisation of the selected carriers around the transmembrane pore. The term anchor and tether are used interchangeably herein.

The anchor may be a polypeptide anchor and/or a hydrophobic anchor that can be inserted into the membrane. In one embodiment, the hydrophobic anchor is a lipid, fatty acid, sterol, carbon nanotube, polypeptide, protein or amino acid, for example cholesterol, palmitate or tocopherol. The anchor may comprise thiol, biotin or a surfactant. In one aspect the anchor may be biotin (for binding to streptavidin), amylose (for binding to maltose binding protein or a fusion protein), Ni-NTA (for binding to poly-histidine or poly-histidine tagged proteins) or peptides (such as an antigen).

In one embodiment, the anchor comprises a linker, or 2, 3, 4 or more linkers. Preferred linkers include, but are not limited to, polymers, such as polynucleotides, polyethylene glycols (PEGs), polysaccharides and polypeptides. These linkers may be linear, branched or circular. For instance, the linker may be a circular polynucleotide. The adapter may hybridise to a complementary sequence on a circular polynucleotide linker. The one or more anchors or one or more linkers may comprise a component that can be cut or broken down, such as a restriction site or a photolabile group. The linker may be functionalised with maleimide groups to attach to cysteine residues in proteins. Suitable linkers are described in WO 2010/086602.

In one embodiment, the anchor is cholesterol or a fatty acyl chain. For example, any fatty acyl chain having a length of from 6 to 30 carbon atom, such as hexadecanoic acid, may be used. Examples of suitable anchors and methods of attaching anchors to adapters are disclosed in WO 2012/164270 and WO 2015/150786. The same methods may be used to attach anchors to the carriers.

### Detector

Any suitable detector can be used in the methods described herein. The detector may be any detector useful in sequencing methods. For example, nanopore sequencing or single-molecule real-time sequencing, e.g. sequencing by synthesis, technology. Preferably, the detector in the methods used herein is a nanopore. Any suitable nanopore can be used in the methods described herein. In one embodiment a nanopore is a transmembrane pore.

A transmembrane pore is a structure that crosses a membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

Any transmembrane pore may be used in the methods provided herein. The pore may be biological or artificial. Suitable pores include, but are not limited to, protein pores, polynucleotide pores and solid state pores. The pore may be a DNA origami pore (Langecker et al., Science, 2012; 338: 932-936). Suitable DNA origami pores are disclosed in WO 2013/083983, WO 2018/011603 and WO 2020/025974.

In one embodiment, the nanopore is a transmembrane protein pore. A transmembrane protein pore is a polypeptide or a collection of polypeptides that permits hydrated ions, such as polynucleotide, to flow from one side of a membrane to the other side of the membrane. In the methods provided herein, the transmembrane protein pore is capable of forming a pore that permits hydrated ions driven by an applied potential to flow from one side of the membrane to the other. The transmembrane protein pore preferably permits polynucleotides to flow from one side of the membrane, such as a triblock copolymer membrane, to the other. The transmembrane protein pore allows a polynucleotide to be moved through the pore.

In one embodiment, the nanopore is a transmembrane protein pore which is a monomer or an oligomer. The pore is preferably made up of several repeating subunits, such as at least 6, at least 7, at least 8, at least 9, at least 10, at least 11, at least 12, at least 13, at least 14, at least 15, or at least 16 subunits. The pore is preferably a hexameric, heptameric, octameric or nonameric pore. The pore may be a homo-oligomer or a hetero-oligomer.

In one embodiment, the transmembrane protein pore comprises a barrel or channel through which the ions may flow. The subunits of the pore typically surround a central axis and contribute strands to a transmembrane β-barrel or channel or a transmembrane α-helix bundle or channel.

Typically, the barrel or channel of the transmembrane protein pore comprises amino acids that facilitate interaction with an analyte, such as a target polynucleotide (as described herein). These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides or nucleic acids.

In one embodiment, the nanopore is a transmembrane protein pore derived from β-barrel pores or α-helix bundle pores. β-barrel pores comprise a barrel or channel that is formed from β-strands. Suitable β-barrel pores include, but are not limited to, -toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. Suitable α-helix bundle pores include, but are not limited to, inner membrane proteins and a outer membrane proteins, such as WZA and ClyA toxin.

In one embodiment the nanopore is a transmembrane pore derived from or based on Msp, α-hemolysin (α-HL), lysenin, CsgG, ClyA, Sp1 or haemolytic protein fragaceatoxin C (FraC).

In one embodiment, the nanopore is a transmembrane protein pore derived from CsgG, e.g. from CsgG from E. coli Str. K-12 substr. MC4100. Such a pore is oligomeric and typically comprises 7, 8, 9 or 10 monomers derived from CsgG. The pore may be a homo-oligomeric pore derived from CsgG comprising identical monomers. Alternatively, the pore may be a hetero-oligomeric pore derived from CsgG comprising at least one monomer that differs from the others. Examples of suitable pores derived from CsgG are disclosed in, for example, WO 2016/034591, WO 2017/149316, WO 2017/149317, WO 2017/149318, WO 2018/211241 and WO 2019/002893.

In one embodiment, the nanopore is a transmembrane pore derived from lysenin. Examples of suitable pores derived from lysenin are disclosed in WO 2013/153359.

In one embodiment, the nanopore is a transmembrane pore derived from or based on α-hemolysin (α-HL). The wild type α-hemolysin pore is formed of 7 identical monomers or sub-units (i.e., it is heptameric). An α-hemolysin pore may be α-hemolysin-NN or a variant thereof. The variant preferably comprises N residues at positions E111 and K147.

In one embodiment, the nanopore is a transmembrane protein pore derived from Msp, e.g. from MspA. Examples of suitable pores derived from MspA are disclosed in WO 2012/107778.

In one embodiment, the nanopore is a transmembrane pore derived from or based on ClyA. Examples of suitable pores derived from ClyA are disclosed in WO 2014/153625.

In one embodiment, the detector is a nanopipette. A nanopipette typically has a diameter of about 10 nm, such as from about 10 nm to about 12, about 15, about 18 or about 20 nm. The nanopipette may be made from a quartz capillary, glass and/or carbon, such glass coated with a carbon layer. Suitable nanopipettes are known in the art.

### Membrane

In embodiments, which comprise the use of a nanopore, the nanopore is typically present in a membrane, for example the nanopore crosses the membrane and/or provides a channel through the membrane. Any suitable membrane may be used and suitable membranes are known in the art.

The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450).

The block copolymer may be a diblock (consisting of two monomer sub-units), but may also be constructed from more than two monomer sub-units to form more complex arrangements that behave as amphipiles. The copolymer may be a triblock, tetrablock or pentablock copolymer. The membrane is preferably a triblock copolymer membrane.

The membrane may, for example, be one of the membranes disclosed in International Application No. WO2014/064443 or WO2014/064444.

The amphiphilic molecules may be chemically-modified or functionalised to facilitate coupling of the anchor. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two dimensional fluids with lipid diffusion rates of approximately 10⁻⁸ cm s⁻¹. This means that the pore and an anchored carrier can typically move within an amphiphilic membrane.

The membrane may be a lipid bilayer. The lipid bilayer may be any lipid bilayer. Suitable lipid bilayers include, but are not limited to, a planar lipid bilayer, a supported bilayer or a liposome. The lipid bilayer is preferably a planar lipid bilayer. Suitable lipid bilayers are disclosed in WO 2008/102121, WO 2009/077734 and WO 2006/100484.

A lipid bilayer may be formed from dried lipids as described in WO 2009/077734. The lipid bilayer may be formed across an opening as described in WO2009/077734.

Any lipid composition that forms a lipid bilayer may be used. The lipid composition is chosen such that a lipid bilayer having the required properties, such surface charge, ability to support membrane proteins, packing density or mechanical properties, is formed. The lipid composition can comprise one or more different lipids. For instance, the lipid composition can contain up to 100 lipids. The lipid composition preferably contains 1 to 10 lipids. The lipid composition may comprise naturally-occurring lipids and/or artificial lipids.

The membrane may comprise a solid state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid state layer may be formed from graphene. Suitable graphene layers are disclosed in WO 2009/035647. If the membrane comprises a solid state layer, the pore is typically present in an amphiphilic membrane or layer contained within the solid state layer, for instance within a hole, well, gap, channel, trench or slit within the solid state layer. The skilled person can prepare suitable solid state/amphiphilic hybrid systems. Suitable systems are disclosed in WO 2009/020682 and WO 2012/005857. Any of the amphiphilic membranes or layers discussed above may be used.

The methods disclosed herein may be carried out using (i) an artificial amphiphilic layer comprising a pore, (ii) an isolated, naturally-occurring lipid bilayer comprising a pore. The artificial amphiphilic layer is typically an artificial triblock copolymer layer. The layer may comprise other transmembrane and/or intramembrane proteins as well as other molecules in addition to the pore. Suitable apparatus and conditions are discussed below. The method of the disclosure is typically carried out in vitro.

Characterising

The methods of the present disclosure comprise characterising the identifier region of a carrier, and determining whether or not the molecule is bound to the molecule-binding region, as described in more detail herein.

The characterisation, and the determining of whether or not the molecule is bound to the molecule-binding region, may be carried out using any suitable detector system. The characterisation, and the determining of whether or not the molecule is bound to the molecule-binding region, may for example be carried out using any apparatus that is suitable for investigating a membrane/pore system in which a pore is inserted into a membrane. The method may be carried out using any apparatus that is suitable for transmembrane pore sensing. For example, the apparatus may comprise a chamber comprising an aqueous solution and a barrier that separates the chamber into two sections. The barrier may have an aperture in which a membrane containing a transmembrane pore is formed. Transmembrane pores are described herein.

The characterisation methods may be carried out using the apparatus described in WO 2008/102120, WO 2010/122293 or WO 2000/028312.

The characterisation methods may involve taking one or more optical or electrical measurements as a carrier moves within the detector, for example a nanopore. The electrical measurement may be measuring the ion current flow through the nanopore, typically by measurement of a current. Possible electrical measurements include: current measurements, impedance measurements, tunneling or electron tunneling measurements (Ivanov A P et al., Nano Lett. 2011 Jan. 12; 1 l(1):279-85), and FET measurements (International Application WO 2005/124888), e.g., voltage FET measurements. In some embodiments, the signal may be electron tunneling across a solid state nanopore or a voltage FET measurement across a solid state nanopore.

Alternatively, ion flow through a nanopore may be measured optically, such as disclosed by Heron et al: J. Am. Chem. Soc. 9 Vol. 131, No. 5, 2009. Methods for optical polymer sequencing using nanopores are described in WO 2016/009180.

The apparatus may also comprise an electrical circuit capable of applying a potential and measuring an electrical signal across the membrane and pore. The characterisation methods may be carried out using a patch clamp or a voltage clamp. The characterisation methods preferably involve the use of a voltage clamp.

The characterisation methods may be carried out on a silicon-based array of wells where each array comprises 128, 256, 512, 1024, 2000, 3000, 4000, 6000, 10000, 12000, 15000 or more wells.

The characterisation methods may involve the measuring of a current flowing through the pore. The method is typically carried out with a voltage applied across the membrane and pore. The voltage used is typically from +2 V to −2 V, typically −400 mV to +400 mV. The voltage used is preferably in a range having a lower limit selected from −400 mV, −300 mV, −200 mV, −150 mV, −100 mV, −50 mV, −20 mV and 0 mV and an upper limit independently selected from +10 mV, +20 mV, +50 mV, +100 mV, +150 mV, +200 mV, +300 mV and +400 mV. The voltage used is more preferably in the range 100 mV to 240 mV and most preferably in the range of 120 mV to 220 mV. It is possible to increase discrimination between different nucleotides by a pore by using an increased applied potential.

The characterisation methods are typically carried out in the presence of any charge carriers, such as metal salts, for example alkali metal salts, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In the exemplary apparatus discussed above, the salt is present in the aqueous solution in the chamber. Potassium chloride (KCl), sodium chloride (NaCl) or caesium chloride (CsCl) is typically used. KCl is preferred. The salt may be an alkaline earth metal salt such as calcium chloride (CaCl₂)). The salt concentration may be at saturation. The salt concentration may be 3M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The characterisation method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of binding/no binding to be identified against the background of normal current fluctuations.

The characterisation methods are typically carried out in the presence of a buffer. In the exemplary apparatus discussed above, the buffer is present in the aqueous solution in the chamber. Any suitable buffer may be used. Typically, the buffer is HEPES. Another suitable buffer is Tris-HCl buffer. The methods are typically carried out at a pH of from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The characterisation methods may be carried out at from 0° C. to 100° C., from 15° C. to 95° C., from 16° C. to 90° C., from 17° C. to 85° C., from 18° C. to 8⁰° C., 19° C. to 70° C., or from 20° C. to 60° C. The characterisation methods are typically carried out at room temperature. The characterisation methods are optionally carried out at a temperature that supports enzyme function, such as about 37° C.

### Carriers, Populations of Carriers, Kits and Systems

Also provided herein are carriers, populations of carriers, kits and systems.

A carrier of the disclosure comprises a single-stranded leader, an identifier region and a molecule-binding region specific for a molecule to be detected, wherein a motor protein is bound to the carrier at a position between the single-stranded leader and the polynucleotide identifier. The leader, identifier region, molecule-binding region and motor protein as described herein above may be applied in any of the embodiments of the carriers, populations of carriers, kits and systems discussed. The carrier may further comprise any of the additional features discussed above.

A population of carriers as described herein, for multiple molecules is also provided. The different carriers in the population may comprise different identifier regions and different molecule-binding regions. For example, each carrier in the population may comprise a unique identifier region and a unique molecule-binding region. There may be multiple copies of each carrier in the population. In other words, the identifier region associated with a molecule-binding region of a carrier may differ from the identifier regions associated with every other molecule-binding region that binds to a different molecule in the population. In some embodiments, the identifier region of a carrier differs from the identifier region of other carriers in the population that bind to different molecules.

The term “associated with” means that the identifier region and one or more molecule-binding regions are present on the same carrier, such that the identifier region may be used to uniquely identify one or more molecule-binding regions on the same carrier. Typically the identifier region is positioned in the carrier such it interacts with the detector under the control of the motor protein prior to the molecule-binding region interacting with the detector. The identifier region may be immediately adjacent to the molecule-binding region or may be separated by a linker. The linker is typically between 2 and about 50, such as between 3 and about 20, preferably between about 5 and about 10 bases in length. The linker may comprise any nucleotides as described herein. One or more spacers may also be positioned between the identifier region and its associated molecule-binding region. The spacer may be present with or without a linker on one or both sides of the spacer.

Also provided is a kit for detecting multiple molecules in a sample, comprising (i) a population of carriers as described herein, and (ii) a motor protein.

Further provided is a system for detecting multiple molecules in a sample, comprising (i) a population of carriers as described herein, (ii) a motor protein, and (iii) a transmembrane pore.

The kit or system may comprise more than one population of carriers as defined herein. In addition to an identifier region associated with a molecule-binding region, each carrier in the population may comprise a further identifier region that is common to all carriers in that population and not present in the carriers of any other population in the kit or system, i.e. the further identifier region is unique to the carriers of that population. Thus the kit or system may comprise two or more populations of carriers, such as 3, 4, 5 or more, for example 10 or more, 20 or more or 50 or more populations of carriers, wherein the carriers in each population comprise a further identifier region that is unique to the carriers of that population. Such a kit or system may be used to analyse multiple samples simultaneously. The molecules present in each of the samples to be detected using the same detector and the further identifier region may be used to determine in which sample(s) a given molecule is present or absent.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016) for definitions and terms of the art. The definitions provided herein should not be construed to have a scope less than understood by a person of ordinary skill in the art.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Nucleotide sequence”, “DNA sequence” or “nucleic acid molecule(s)” as used herein refers to a polymeric form of nucleotides of any length, either ribonucleotides or deoxyribonucleotides. This term refers only to the primary structure of the molecule. Thus, this term includes double- and single-stranded DNA, and RNA. The term “nucleic acid” as used herein, is a single or double stranded covalently-linked sequence of nucleotides in which the 3′ and 5′ ends on each nucleotide are joined by phosphodiester bonds. The polynucleotide may be made up of deoxyribonucleotide bases or ribonucleotide bases. Nucleic acids may be manufactured synthetically in vitro or isolated from natural sources. Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA). Sizes of nucleic acids, also referred to herein as “polynucleotides” are typically expressed as the number of base pairs (bp) for double stranded polynucleotides, or in the case of single stranded polynucleotides as the number of nucleotides (nt). One thousand bp or nt equal a kilobase (kb). Polynucleotides of less than around 40 nucleotides in length are typically called “oligonucleotides” and may comprise primers for use in manipulation of DNA such as via polymerase chain reaction (PCR).

The term “amino acid” in the context of the present disclosure is used in its broadest sense and is meant to include organic compounds containing amine (NH₂) and carboxyl (COOH) functional groups, along with a side chain (e.g., a R group) specific to each amino acid. In some embodiments, the amino acids refer to naturally occurring L α-amino acids or residues. The commonly used one and three letter abbreviations for naturally occurring amino acids are used herein: A=Ala; C=Cys; D=Asp; E=Glu; F=Phe; G=Gly; H=His; I=Ile; K=Lys; L=Leu; M=Met; N=Asn; P=Pro; Q=Gln; R=Arg; S=Ser; T=Thr; V=Val; W=Trp; and Y=Tyr (Lehninger, A. L., (1975) Biochemistry, 2d ed., pp. 71-92, Worth Publishers, New York). The general term “amino acid” further includes D-amino acids, retro-inverso amino acids as well as chemically modified amino acids such as amino acid analogues, naturally occurring amino acids that are not usually incorporated into proteins such as norleucine, and chemically synthesised compounds having properties known in the art to be characteristic of an amino acid, such as 3-amino acids. For example, analogues or mimetics of phenylalanine or proline, which allow the same conformational restriction of the peptide compounds as do natural Phe or Pro, are included within the definition of amino acid. Such analogues and mimetics are referred to herein as “functional equivalents” of the respective amino acid. Other examples of amino acids are listed by Roberts and Vellaccio, The Peptides: Analysis, Synthesis, Biology, Gross and Meiehofer, eds., Vol. 5 p. 341, Academic Press, Inc., N.Y. 1983, which is incorporated herein by reference.

The terms “polypeptide”, and “peptide” are interchangeably used herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. Polypeptides can also undergo maturation or post-translational modification processes that may include, but are not limited to: glycosylation, proteolytic cleavage, lipidization, signal peptide cleavage, propeptide cleavage, phosphorylation, and such like. A peptide can be made using recombinant techniques, e.g., through the expression of a recombinant or synthetic polynucleotide. A recombinantly produced peptide it typically substantially free of culture medium, e.g., culture medium represents less than about 20%, more preferably less than about 10%, and most preferably less than about 5% of the volume of the protein preparation.

The term “protein” is used to describe a folded polypeptide having a secondary or tertiary structure. The protein may be composed of a single polypeptide, or may comprise multiple polypeptides that are assembled to form a multimer. The multimer may be a homooligomer, or a heterooligmer. The protein may be a naturally occurring, or wild type protein, or a modified, or non-naturally, occurring protein. The protein may, for example, differ from a wild type protein by the addition, substitution or deletion of one or more amino acids.

A “variant” of a protein encompass peptides, oligopeptides, polypeptides, proteins and enzymes having amino acid substitutions, deletions and/or insertions relative to the unmodified or wild-type protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

For all aspects and embodiments of the present invention, a “variant” has at least 50%, 60%, 70%, 80%, 90%, 95% or 99% complete sequence identity to the amino acid sequence of the corresponding wild-type protein. Sequence identity can also be to a fragment or portion of the full length polynucleotide or polypeptide. Hence, a sequence may have only 50% overall sequence identity with a full length reference sequence, but a sequence of a particular region, domain or subunit could share 80%, 90%, or as much as 99% sequence identity with the reference sequence.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified”, “mutant” or “variant” refers to a gene or gene product that displays modifications in sequence (e.g., substitutions, truncations, or insertions), post-translational modifications and/or functional properties (e.g., altered characteristics) when compared to the wild-type gene or gene product. It is noted that naturally occurring mutants can be isolated; these are identified by the fact that they have altered characteristics when compared to the wild-type gene or gene product. Methods for introducing or substituting naturally-occurring amino acids are well known in the art. For instance, methionine (M) may be substituted with arginine (R) by replacing the codon for methionine (ATG) with a codon for arginine (CGT) at the relevant position in a polynucleotide encoding the mutant monomer. Methods for introducing or substituting non-naturally-occurring amino acids are also well known in the art. For instance, non-naturally-occurring amino acids may be introduced by including synthetic aminoacyl-tRNAs in the IVTT system used to express the mutant monomer. Alternatively, they may be introduced by expressing the mutant monomer in E. coli that are auxotrophic for specific amino acids in the presence of synthetic (i.e. non-naturally-occurring) analogues of those specific amino acids. They may also be produced by naked ligation if the mutant monomer is produced using partial peptide synthesis. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

As described in more detail herein, a mutant or modified protein, monomer or peptide can be chemically modified in any way and at any site. A mutant or modified monomer or peptide is preferably chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The mutant of modified protein, monomer or peptide may be chemically modified by the attachment of any molecule. For instance, the mutant of modified protein, monomer or peptide may be chemically modified by attachment of a dye or a fluorophore.

The present invention is described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Similarly, it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

It should be appreciated that “embodiments” of the disclosure can be specifically combined together unless the context indicates otherwise. The specific combinations of all disclosed embodiments (unless implied otherwise by the context) are further disclosed embodiments of the claimed invention.

In addition as used in this specification and the appended claims, the singular forms “a”, “an”, and “the” include plural referents unless the content clearly dictates otherwise. Thus, for example, reference to “a polynucleotide” includes two or more polynucleotides, reference to “a motor protein” includes two or more such proteins, reference to “a helicase” includes two or more helicases, reference to “a monomer” refers to two or more monomers, reference to “a pore” includes two or more pores and the like.

All publications, patents and patent applications cited herein, whether supra or infra, are hereby incorporated by reference in their entirety.

It is to be understood that although particular embodiments, specific configurations as well as materials and/or molecules, have been discussed herein for methods according to the present invention, various changes or modifications in form and detail may be made without departing from the scope and spirit of this invention. The following examples are provided to better illustrate particular embodiments, and they should not be considered limiting the application. The application is limited only by the claims.

### EXAMPLES

In the examples below, the carrier was synthesized in two parts and the parts ligated together. It is also envisaged that the carrier in its entirety may be synthesized as a single unit without need for ligation.

### Example 1

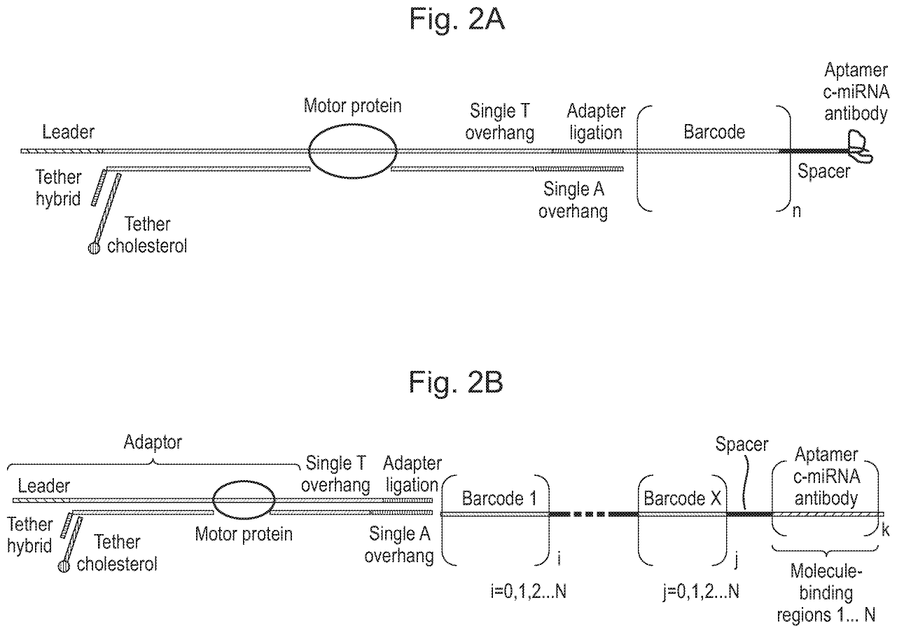

This example describes some of the carriers designed to date. The provided sequences do not include the leader. The leader may be added to the provided sequences through the use of a ligation C-strand (e.g. CCCAGCGGAACTAGGA (SEQ ID NO: 157)), which also comprises a region complementary to the 3′ end of a polynucleotide comprising the leader and the stall region for the motor protein, as shown in FIG. 2A.

The provided carrier sequences may bind to molecules including proteins (such as thrombin, and the SARS-CoV-2 spike protein), neurotransmitters (such as serotonin and dopamine) and miRNAs. The identifier region for each carrier differs from the carrier for every other carrier. It is foreseen that different molecule-binding regions on different carriers that bind to the same molecule may use the same barcode.

The sequences comprise a 5′ ligation strand, an identifier sequence or barcode (underlined), optionally a spacer region comprising iSpC3 or iSp18 spacers, and a molecule-binding region (italics). The molecule-binding regions in this example are aptamers or complementary sequences of miRNAs.

Thrombin
(SEQ ID NO: 29)
/5Phos/CCTAGTTCCGCTGGGGAGCTAGCATACGTGTCTAACACTGCACAGATGAT
(SEQ ID NO: 158)
/iSpC3/iSpC3/GGTTGGTGTGGTTGG
(SEQ ID NO: 30)
/5Phos/CCTAGTTCCGCTGGGATATAGGCATAAAGTAAAATCGTACCAACTCAATC
(SEQ ID NO: 159)
/iSpC3/iSpC3/GGTTGGTGTGGTTGG
(SEQ ID NO: 31)
/5Phos/CCTAGTTCCGCTGGGCTCGGCGTTGTGTGTCAAATGGCGTAGATCTGGAT
(SEQ ID NO: 160)
/iSpC3/iSpC3/GGTTGGTGTGGTTGG
(SEQ ID NO: 32)
/5Phos/CCTAGTTCCGCTGGGCACAGCCCCATGTAACCCAT
(SEQ ID NO: 161)
/iSp18/iSp18/AGTCCGTGGTAGGGCAGGTTGGGGTGACT
Serotonin
(SEQ ID NO: 33)
/5Phos/CCTAGTTCCGCTGGGACTAGATAAAAGGAAGGGAGCACAGTAACGTCGTT
(SEQ ID NO: 162)
/iSpC3/iSpC3/CGACTGGTAGGCAGATAGGGGAAGCTGATTCGATG
Dopamine
(SEQ ID NO: 34)
/5Phos/CCTAGTTCCGCTGGGTCTCCCGTATCCGTGGCTAAACGCCTTCAATCTTA
(SEQ ID NO: 163)
/iSpC3/iSpC3/GGATATTGCGCGATTCCGGTCGGCAGCTTAGGAAGTGCGGTGTC
SARS-CoV-2,SPROTEIN
(SEQ ID NO: 35)
/5Phos/CCTAGTTCCGCTGGGACCTTTTCTAGGATGGAACATTCTA
(SEQ ID NO: 164)
/iSpC3/iSpC3/CACGCATAACGTCTTGCGGGGCGGCGGGTTGAGAGGATGTCGGG
TGGTTATGCGTG
miRNAs(barcodes11-20)
Adapter_barcode11_c-has-miR-497-5p
(SEQ ID NO: 36)
/5Phos/CCTAGTTCCGCTGGGTGCTACTCTTCCTCATAAGCAGTCCGGTGTATCGAT
(SEQ ID NO: 165)
/iSpC3/iSpC3/ACAAACCACAGTGTGCTGCTG
Adapter_barcode12_c-has-miR-27b-5p
(SEQ ID NO: 37)
/5Phos/CCTAGTTCCGCTGGGATCGCTACGCCTTCGGCTCGTAATCATAGTCGAGT
(SEQ ID NO: 166)
/iSpC3/iSpC3/GTTCACCAATCAGCTAAGCTCT
Adapter_barcode13_c-has-miR-21-5p
(SEQ ID NO: 38)
/5Phos/CCTAGTTCCGCTGGGAGCTCAGAGCAGGTCACTCAAGATACGAGCTGCGT
(SEQ ID NO: 167)
/iSpC3/iSpC3/TCAACATCAGTCTGATAAGCTA
Adapter_barcode14_c-has-miR-221-5p
(SEQ ID NO: 39)
/5Phos/CCTAGTTCCGCTGGGGTAAGTCTGCATCAGCGCGCGGCTGTGCGAGGATA
(SEQ ID NO: 168)
/iSpC3/iSpC3/AAATCTACATTGTATGCCAGGT
Adapter_barcode15_c-has-miR-30d-5p
(SEQ ID NO: 40)
/5Phos/CCTAGTTCCGCTGGGCTACGACAGTACGCTAGCAAGGATAGACACTACGA
(SEQ ID NO: 169)
/iSpC3/iSpC3/CTTCCAGTCGGGGATGTTTACA
Adapter_barcode16_c-has-miR-30c-5p/
(SEQ ID NO: 41)
5Phos/CCTAGTTCCGCTGGGTACTGAACACAAGTTCGTCGTCGAGCAATCACAAT
(SEQ ID NO: 170)
/iSpC3/iSpC3/GCTGAGAGTGTAGGATGTTTACA
Adapter_barcode17_c-has-miR-133a-5p/
(SEQ ID NO: 42)
5Phos/CCTAGTTCCGCTGGGAGTCTACCATTACTTGGATCGGATTAGCCTCACTC
(SEQ ID NO: 171)
/iSpC3/iSpC3/ATTTGGTTCCATTTTACCAGCT
Adapter_barcode18_c-has-miR-208a-5p/
(SEQ ID NO: 43)
5Phos/CCTAGTTCCGCTGGGTGCACGAGTGCGTGTCAACCGTCCAGATGCTCGTG
(SEQ ID NO: 172)
/iSpC3/iSpC3/GTATAACCCGGGCCAAAAGCTC
Adapter_barcode19_c-has-miR-181b-5p/
(SEQ ID NO: 44)
5Phos/CCTAGTTCCGCTGGGCTAGTGCGCAGTTGTCTCGGCGGAGTTGAGACTGA
(SEQ ID NO: 173)
/iSpC3/iSpC3/ACCCACCGACAGCAATGAATGTT
Adapter_barcode20_c-has-miR-29a-3p/
(SEQ ID NO: 45)
5Phos/CCTAGTTCCGCTGGGGATCATGGTAGTCTTCAAGATCGAGTATGTCTGTC
(SEQ ID NO: 174)
/iSpC3/iSpC3/TAACCGATTTCAGATGGTGCTA
miRNAS(barcodes 21-22)
without C3 spacer
Adapter_barcode21_c-has-miR-30c-5p/
(SEQ ID NO: 46)
5Phos/CCTAGTTCCGCTGGGTACTGAACACAAGTTCGTCGTCGAGCAATCACAAT
GCTGAGAGTGTAGGATGTTTACA
Adapter_barcode22_c-has-miR-29a-3p/
(SEQ ID NO: 47)
5Phos/CCTAGTTCCGCTGGGCGTGAAGAGAGTTTCATAATACGTCCAGCCGCAT
GTAACCGATTTCAGATGGTGCTA

### Example 2

This example describes the identification of individual carriers by sequencing and demultiplexing of their unique barcodes. This example also describes the identification of the carriers that are bound to an analyte by stalling analysis.

### Methods

Barcodes as described in Example 1 (total concentration 30 nM, equal concentration for each) were incubated with ligation c-strand in a molar ratio of 1:3 in nuclease-free water for 1 h. The hybridization was initiated by centrifuging at 4° C. for 3 minutes, followed by incubating at room temperature for 1 hour. The resultant mixture was mixed with 10 nM adapter, and the ligation was performed by adding an equal volume of TA ligase master mix (New England Biolabs), centrifuged at 4° C. for 3 minutes to thoroughly mix different components while preserving the ligase at low temperature. After incubating at room temperature for 20 minutes, 1.4 times of the total volume of Ampure XP beads (Beckmann Coutler) was added to absorb nucleic acids for further purification. The beads were washed with short fragment buffer (Oxford Nanopore Technologies) to selectively remove excess amount of unhybridised ligation c-strands and barcodes that were not ligated. After purification, the beads were washed with nuclease-free water to wash off purified nucleic acids containing ligated motor protein-barcode complexes. The final solution of sequencing experiments was made up by the eluent, sequencing buffer, tether (100 nM), nuclease-free water, and incubated for at least 30 minutes with certain concentrations of targeted analytes.

All experiments were analysed with the Nanopore App, which is a pre-existing custom-written MATLAB code (developed by Joshua Edel, between 2006-2021). FAST5 sequencing files were uploaded into the app, where they were further processed. The app allowed all 512 channels of the MinION to be uploaded and analysed individually or in bulk.

Translocation events were detected by using a thresholding algorithm. First, a linear baseline was set between −0.16 and −2 nA, depending on the experiment. A step offset of 1.8 was used to define the start of the events (green line). If the events cross the threshold of std 50 (black line), they are detected by the algorithm. Those events are further analysed by filtering for events between 0.025 s and 10 s. All events within this time frame are defined as events. In addition, the algorithm is used to locate the C3 or other spacer elements within the strand design, that are used as alignment markers. In this way, the presence of the spacer is not essential for the detection of the presence or absence of a molecule on the carrier.

GUPPY or/and MinKNOW software (Oxford Nanopore Technologies) were used to sequence and basecall the events detected in the previous step.

After basecalling the raw read signals, the sequenced events were aligned to the reference sequences, e.g. barcode sequences 1-10. The algorithm assigns a barcode to the event of the highest alignment score, if the following points are true:
1) alignment score needs to be higher than 50,
2) the difference of the max alignment score to the second highest score needs to be at least 5,
3) the difference of the max alignment score to the mean alignment score of other barcodes needs to be at least 10,
4) p-value has to be <0.0001.

Only when all criteria were fulfilled, the max alignment was classified as barcode. If not, the event was removed and not considered for further analysis. False positives were removed by calculating the p-value of the highest alignment score compared to the rest of the population.

For the stalling analysis, the C3 parts, as well as the start and end of the event were defined. This was important to calculate the mean translocation time of all sequenced reads. If the translocation time of an event was significantly longer (typically used moving std 100 bins) compared to the mean, the event was classified as stalled. Moreover, each stalling needed to be greater than 20 bins to be considered as stalling.

Identification of Individual Carriers by Sequencing and Demultiplexing of their Unique Barcodes.

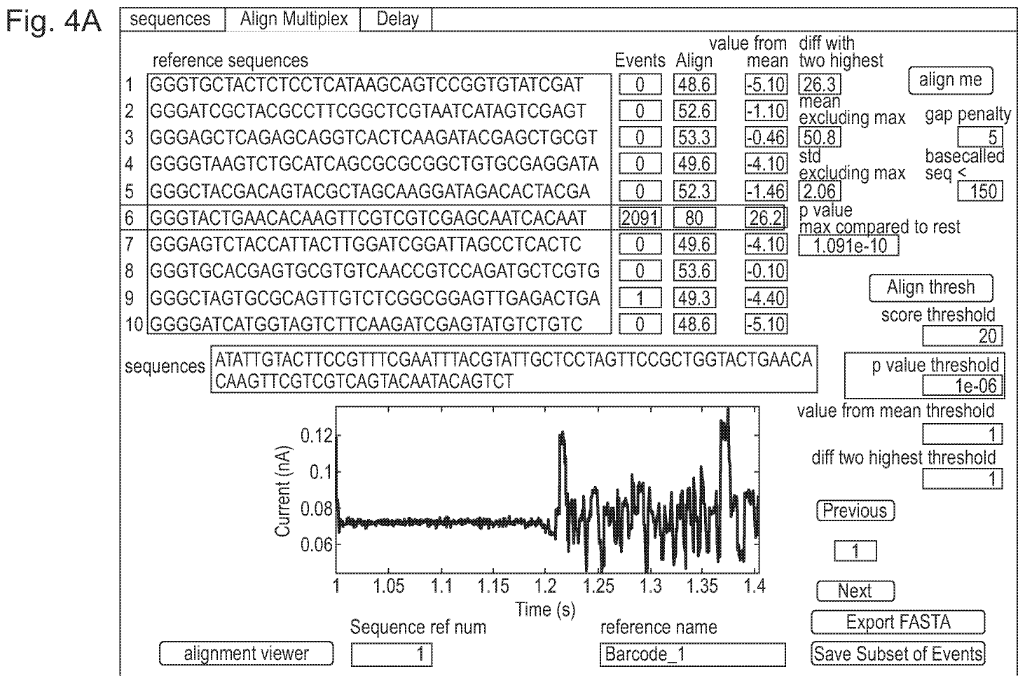

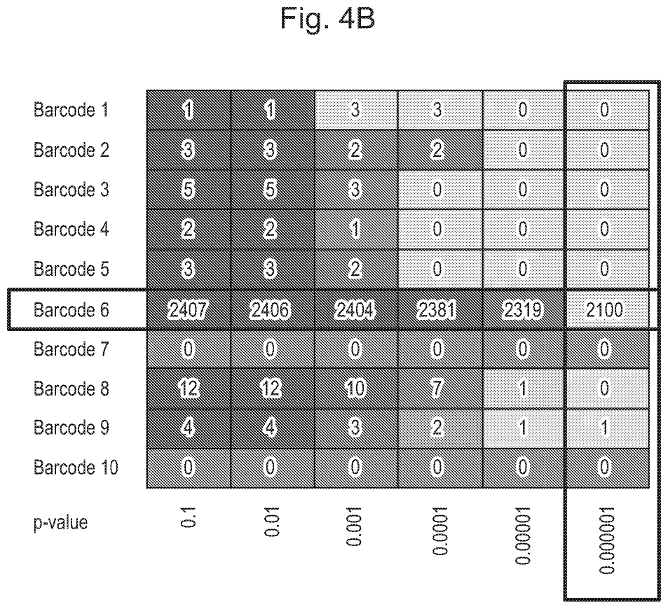

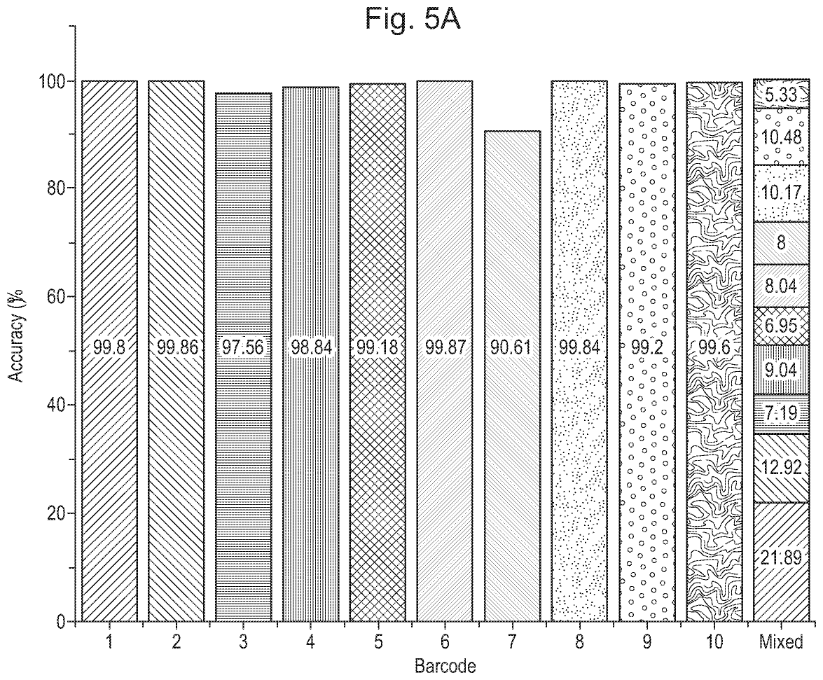

All sequences were basecalled was described above and aligned to reference sequences, which are the barcode sequences. The max alignment score was used to classify the barcode. If the max alignment score and the second highest were too close together, the event wouldn't be classified. Furthermore, the p-value was used to classify events and remove false positive classifications. With this method, an accuracy of 99.95% is achieved at identifying individual barcodes, with 86% of all recorded events being used and classified, meaning that relatively little data was wasted when compared to previous methods in the art (FIG. 4).

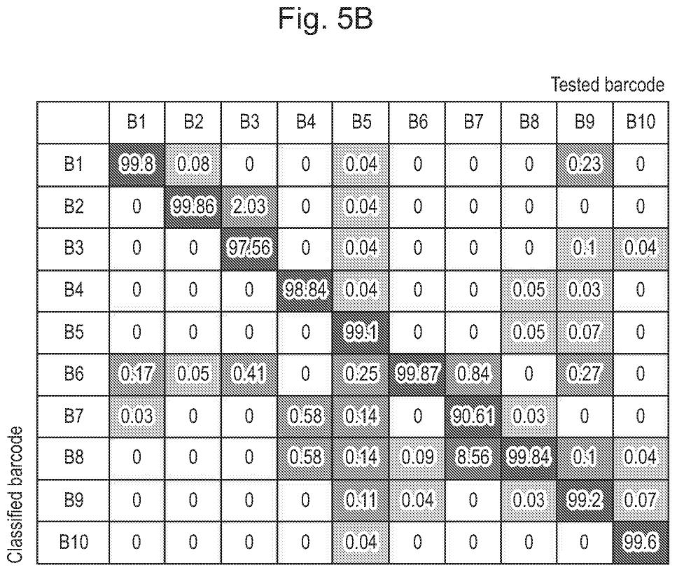

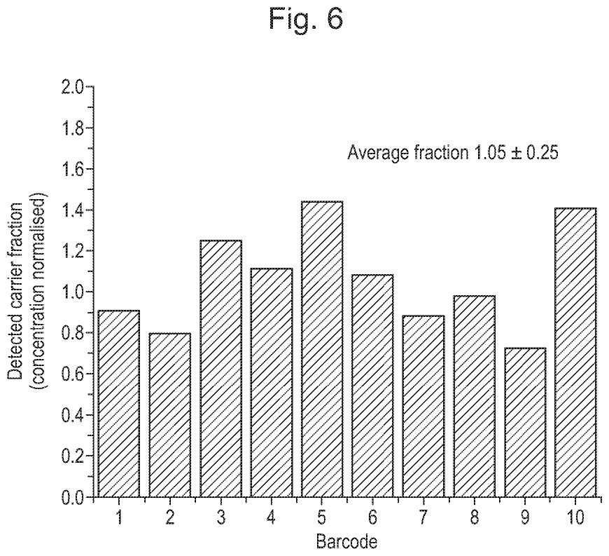

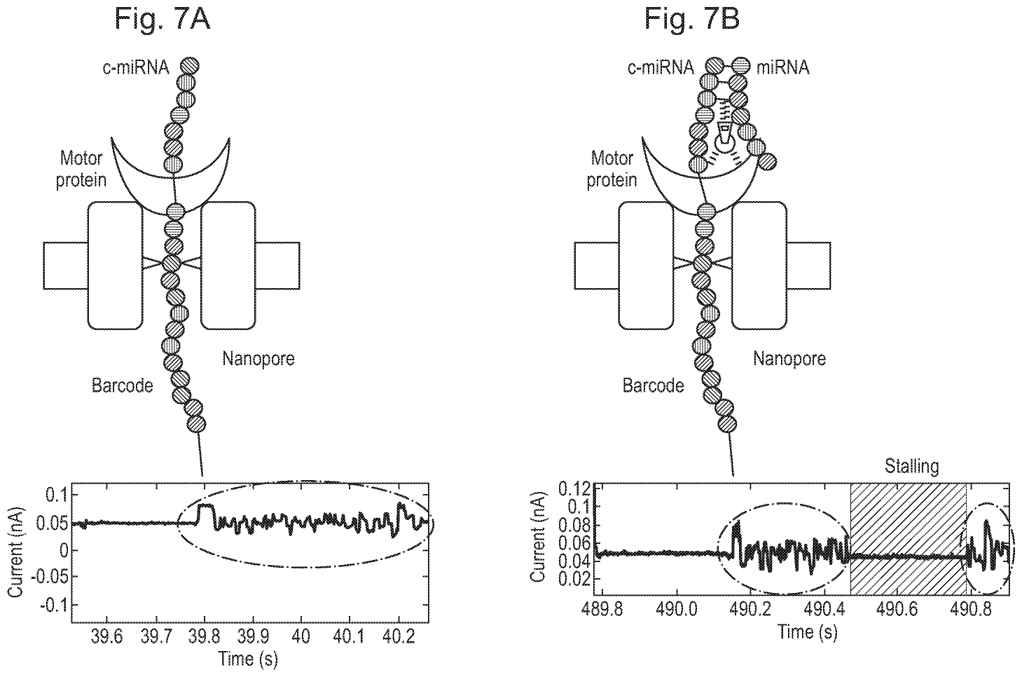

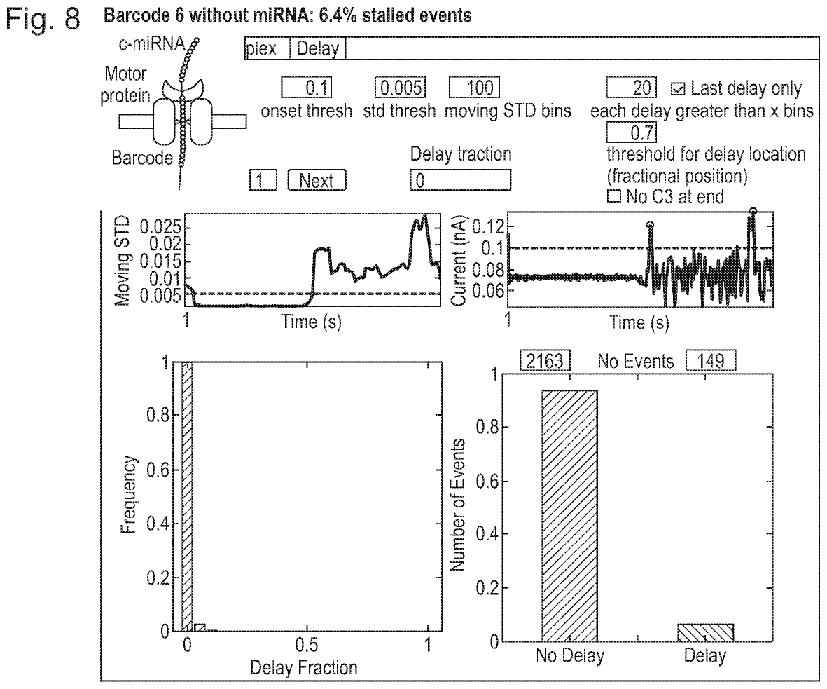

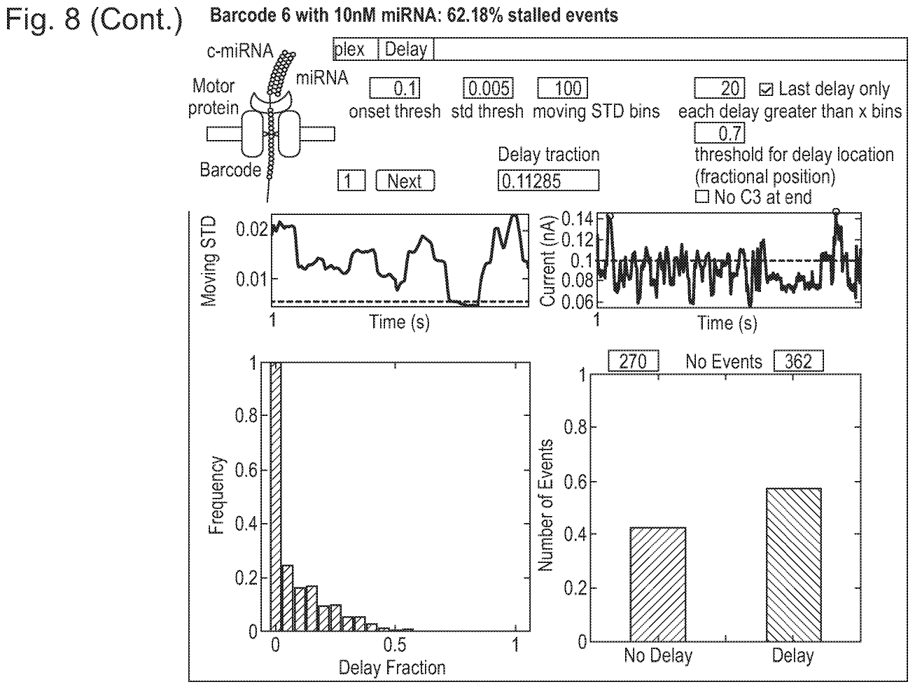

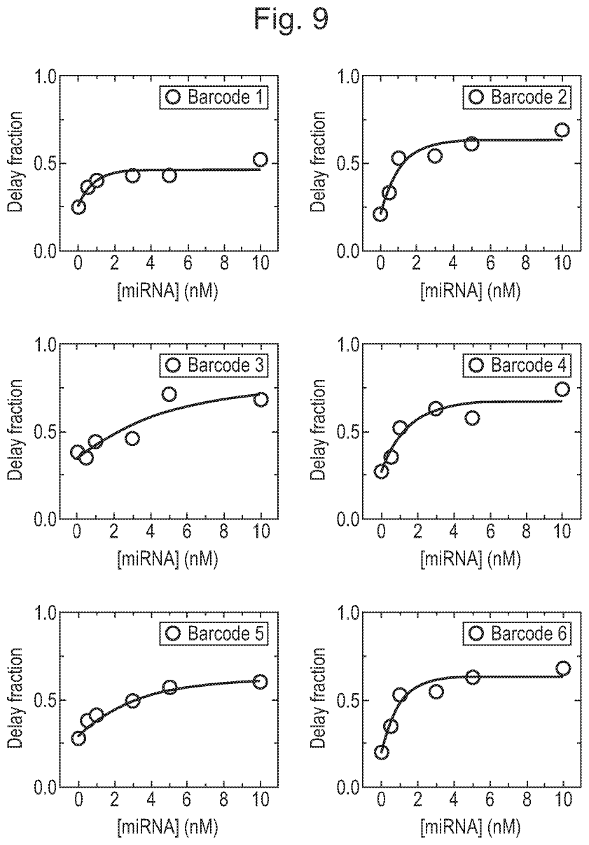

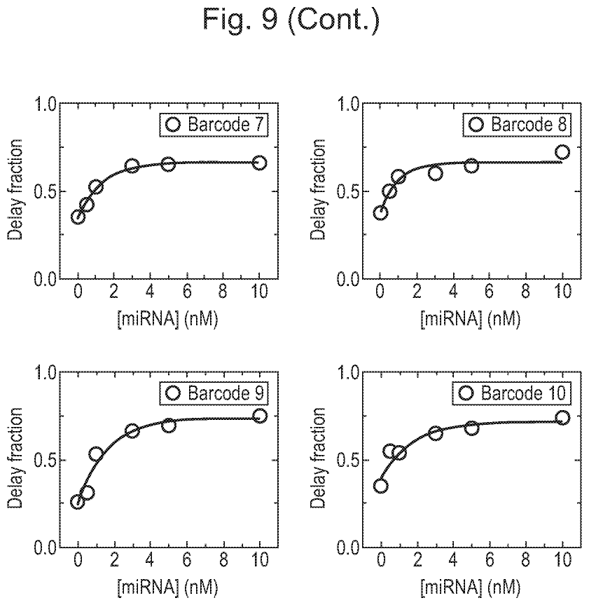

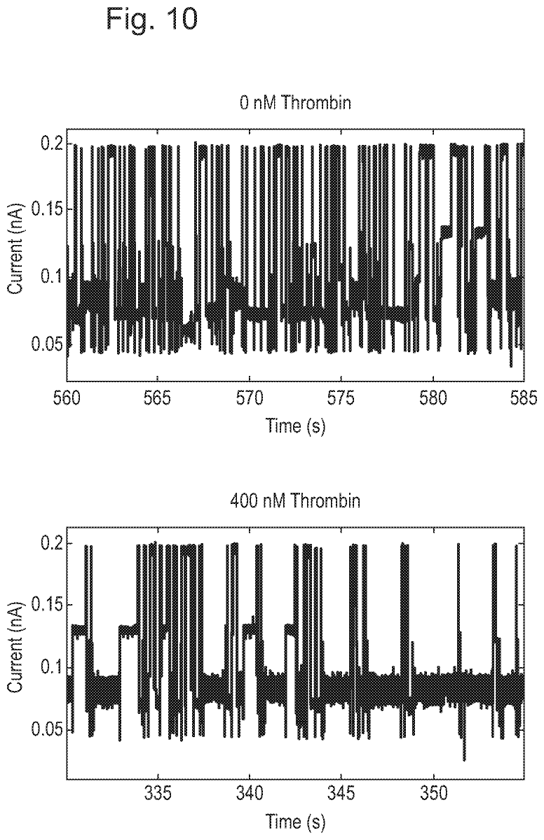

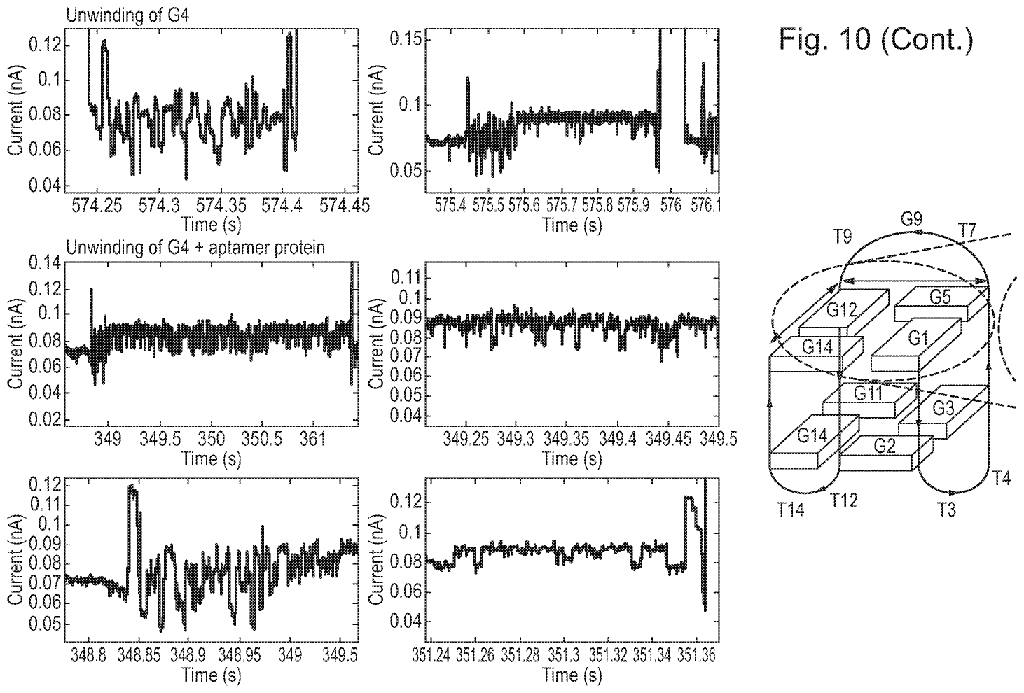

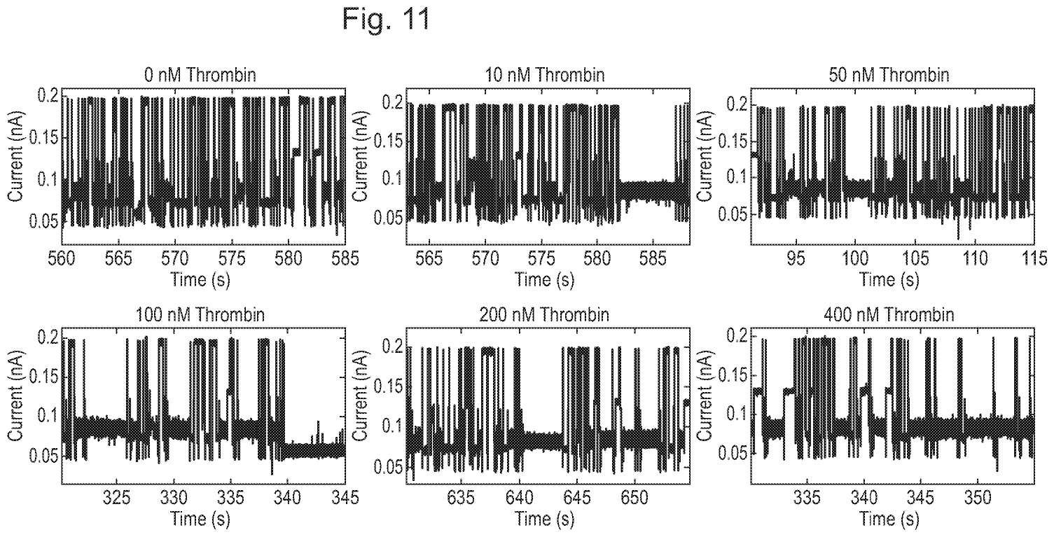

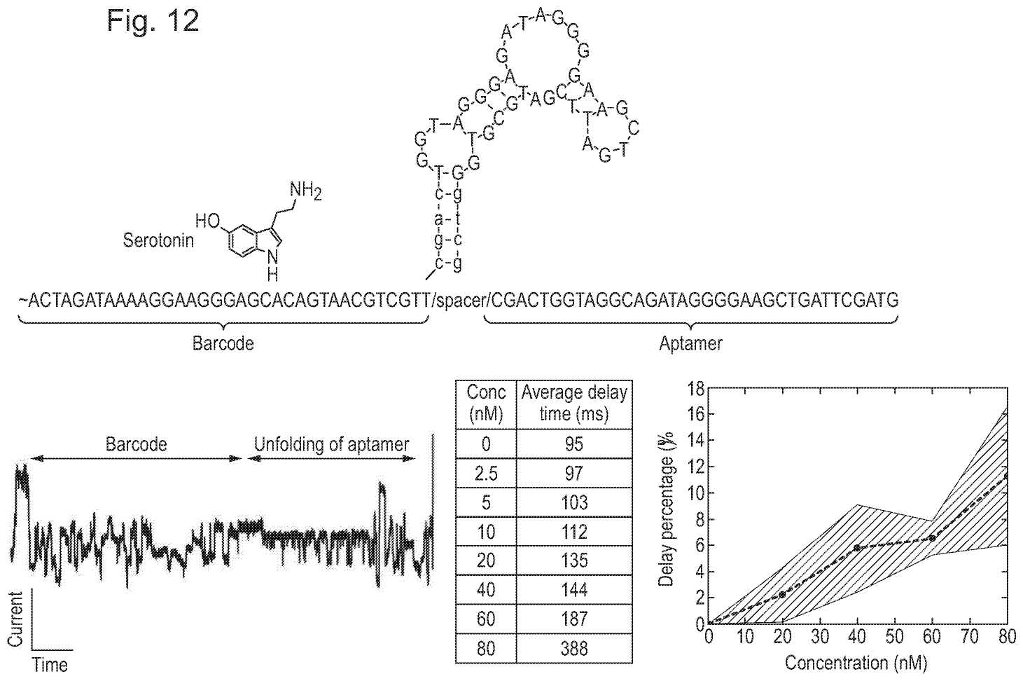

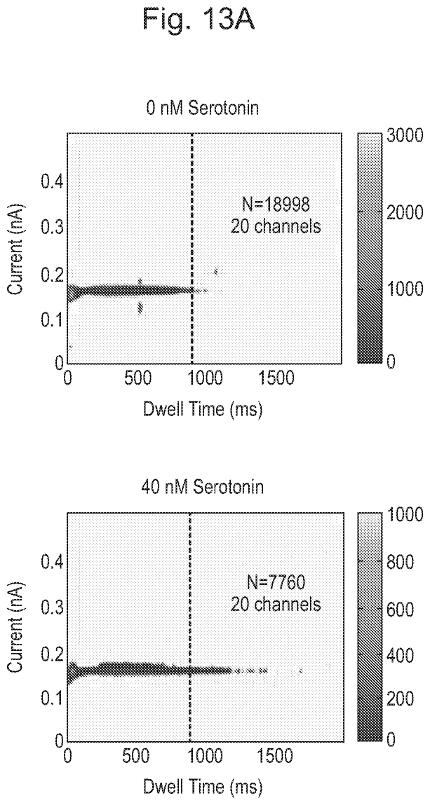

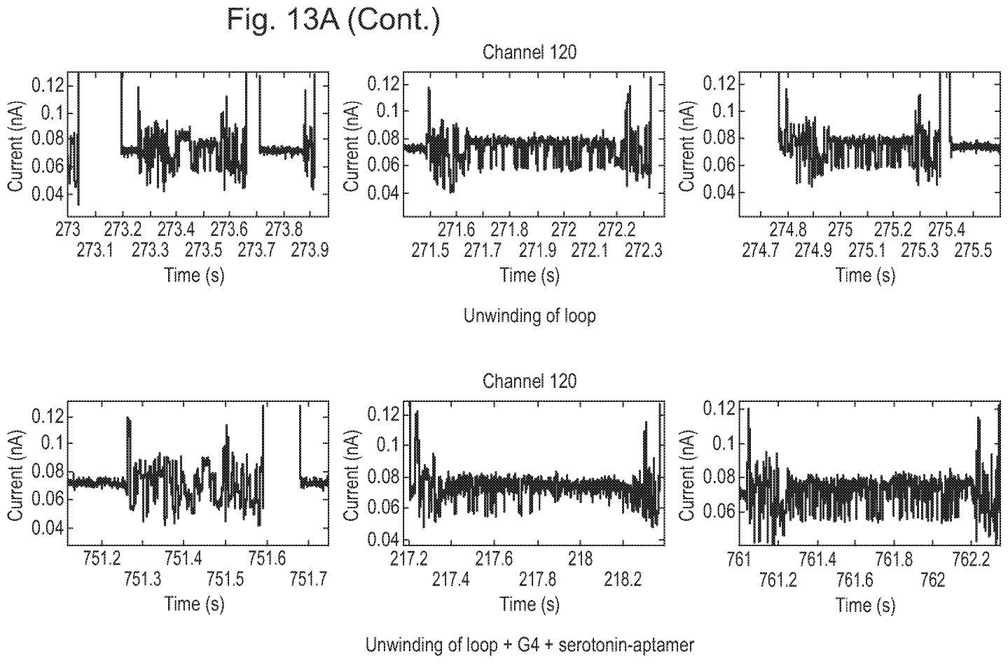

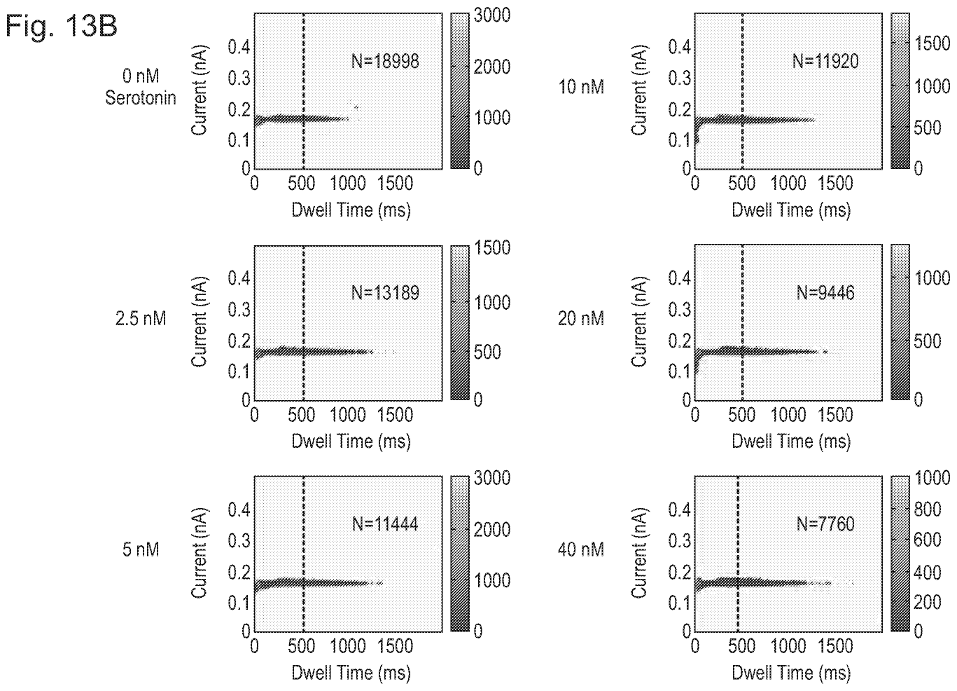

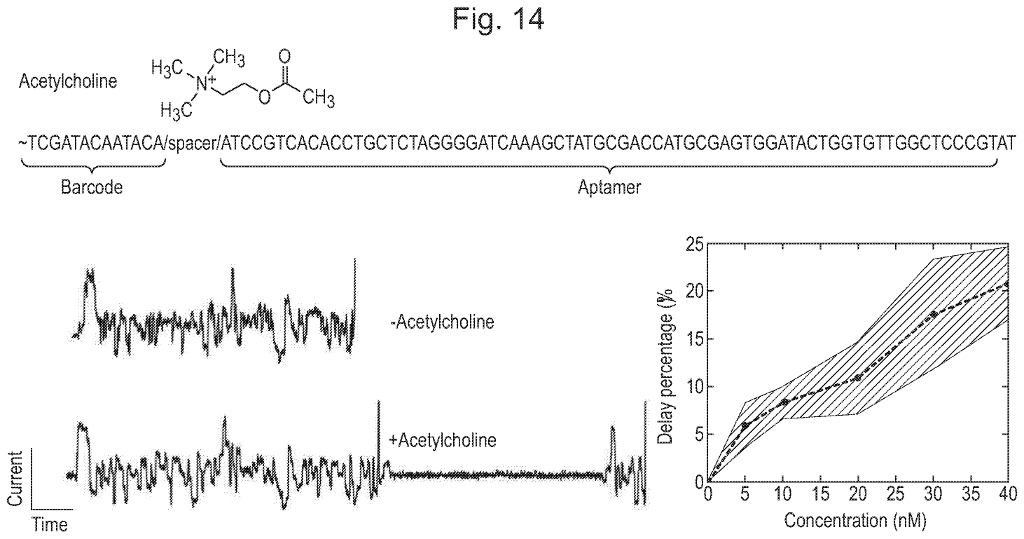

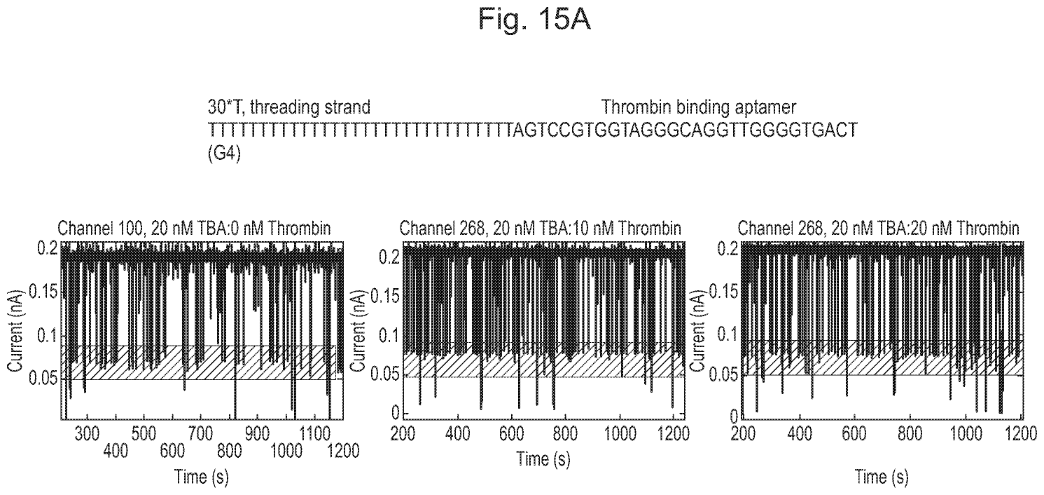

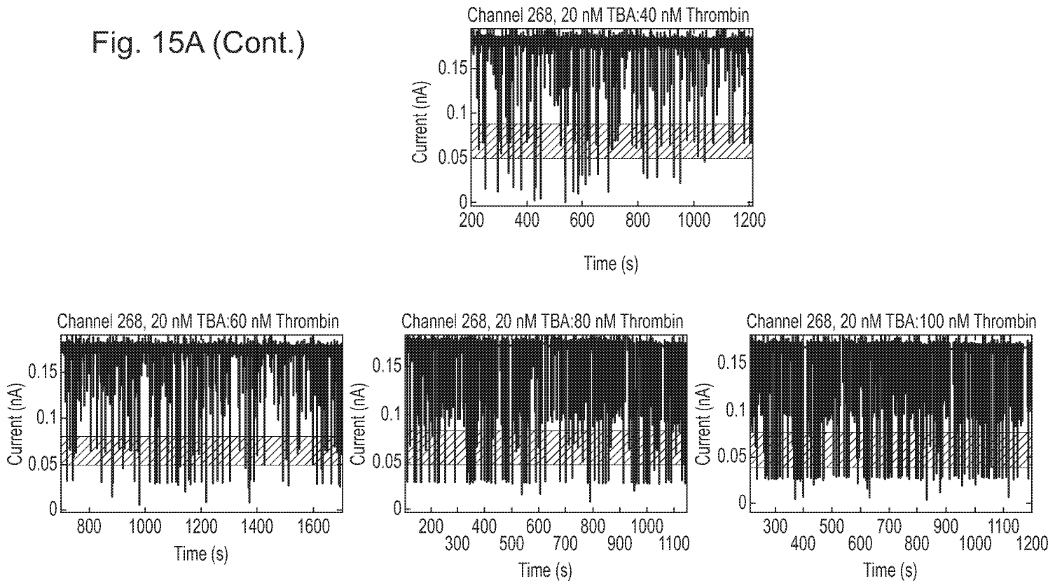

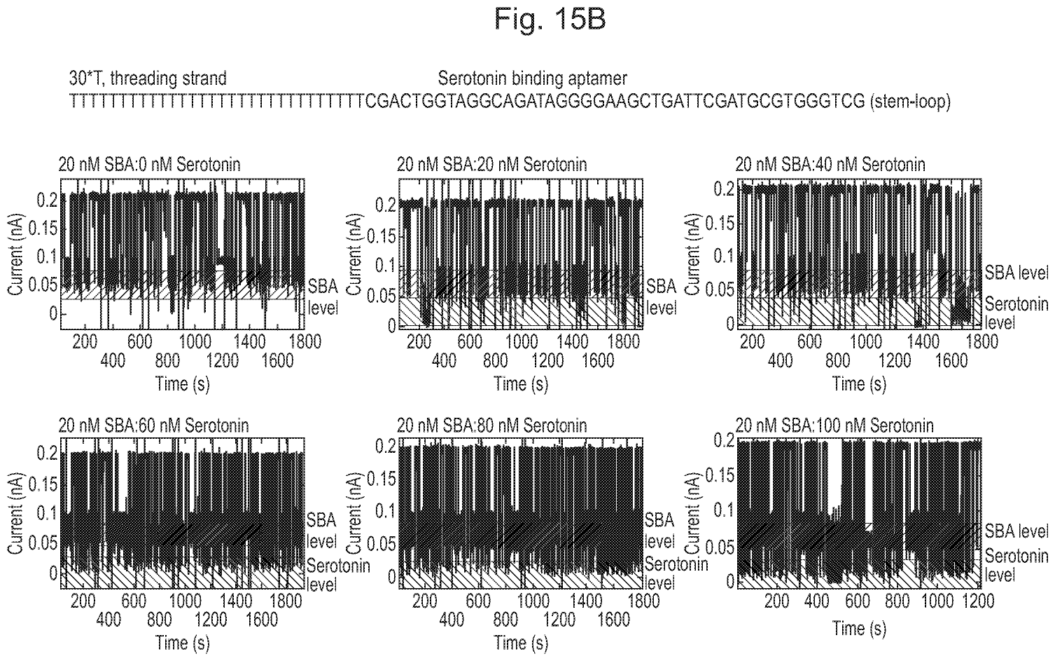

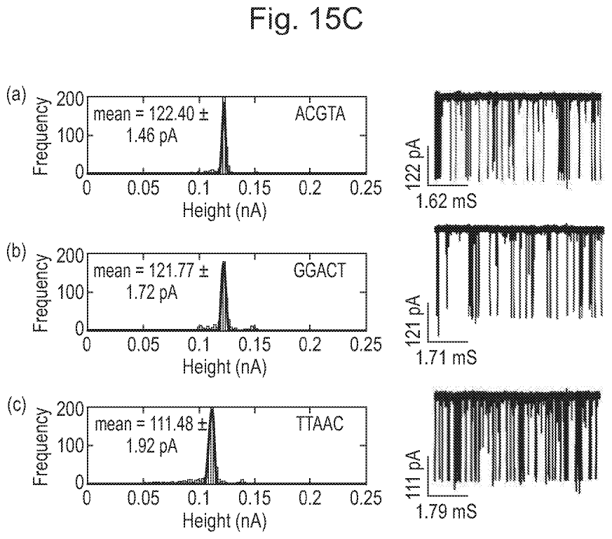

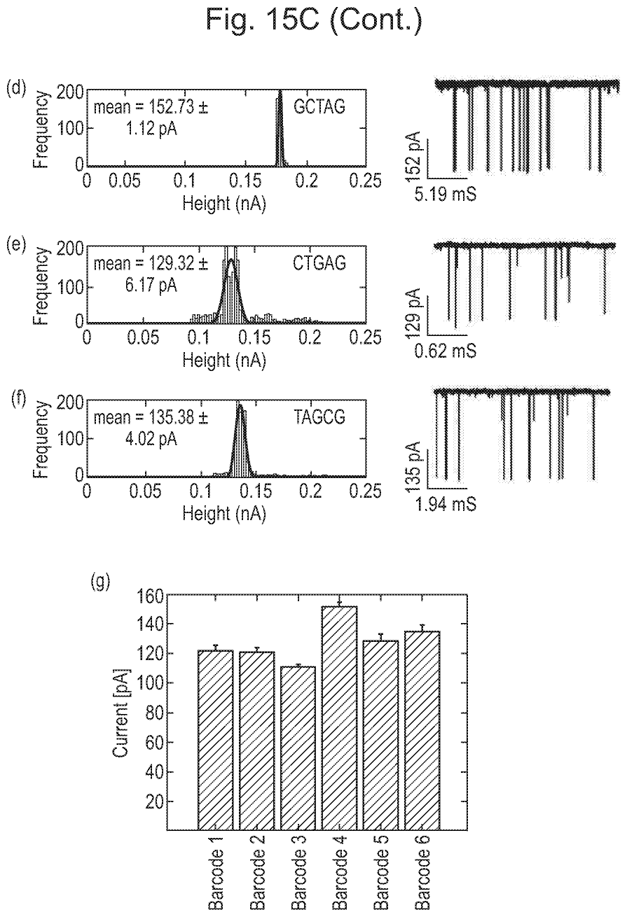

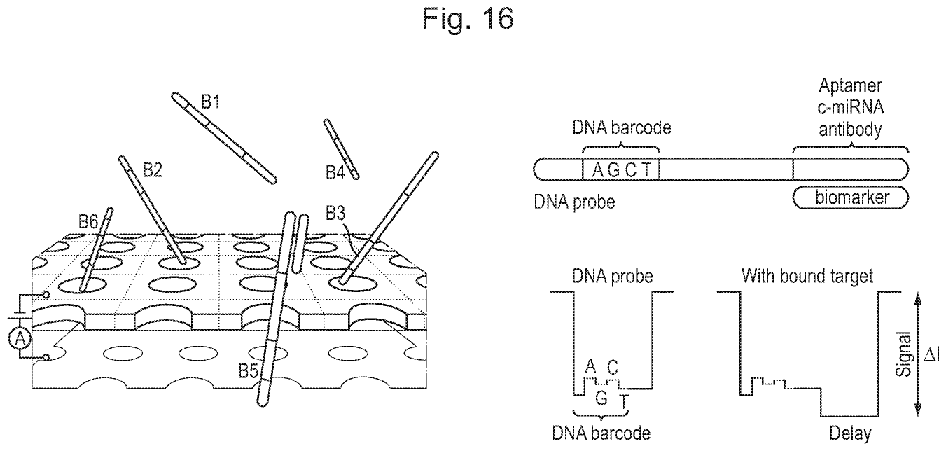

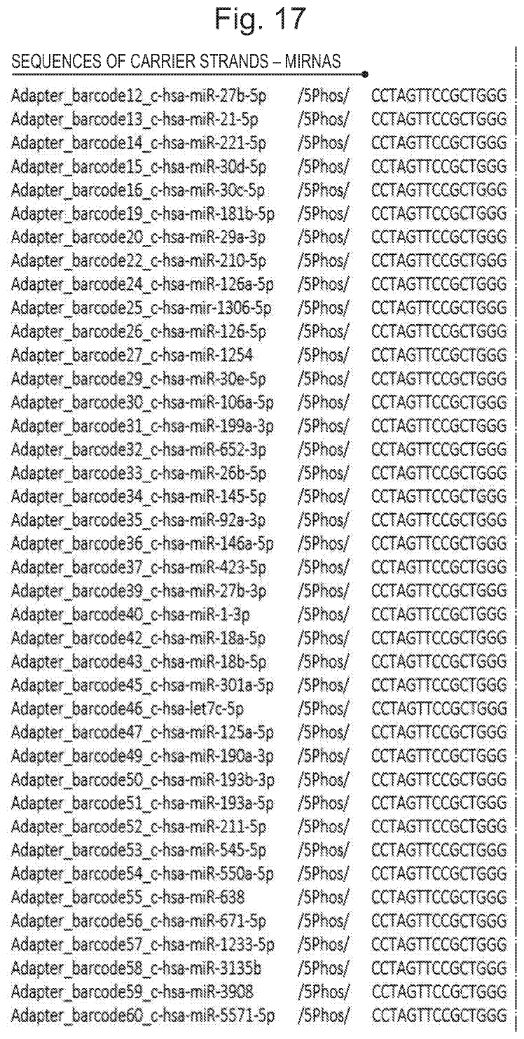

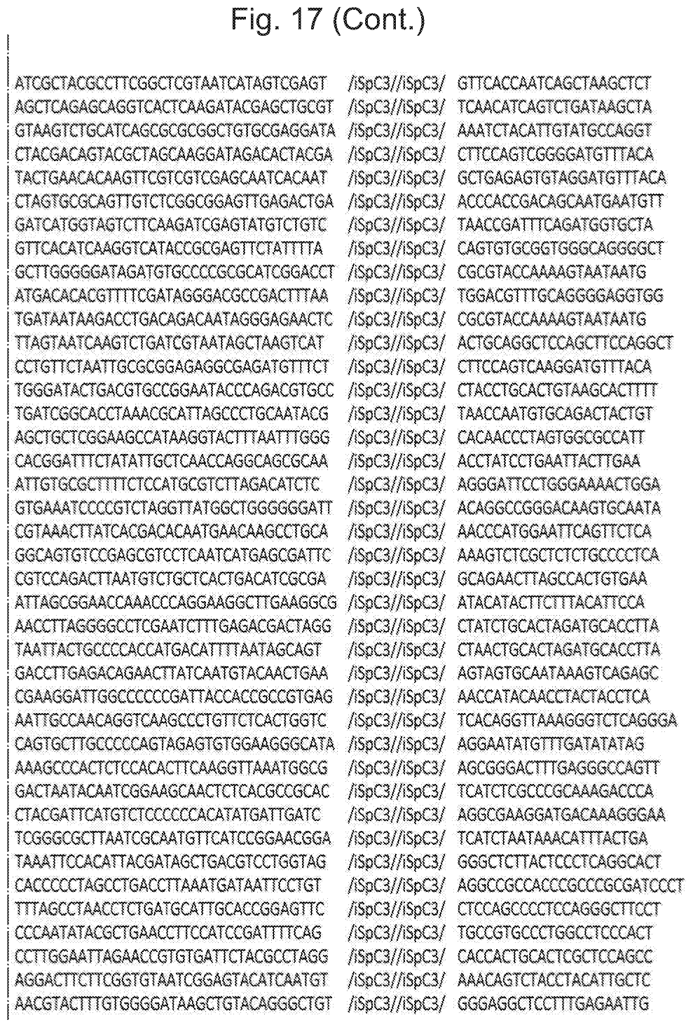

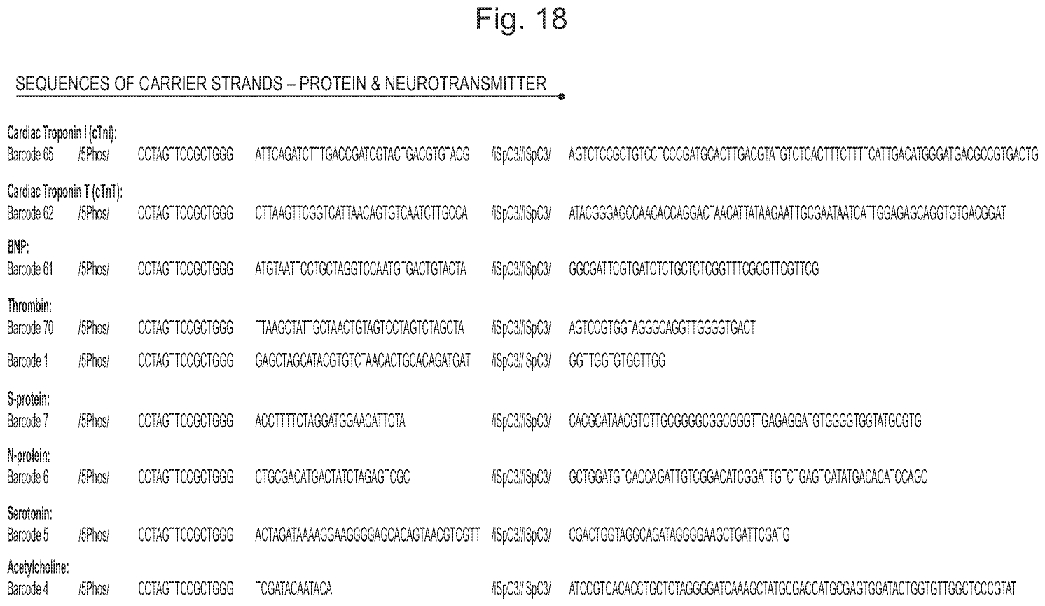

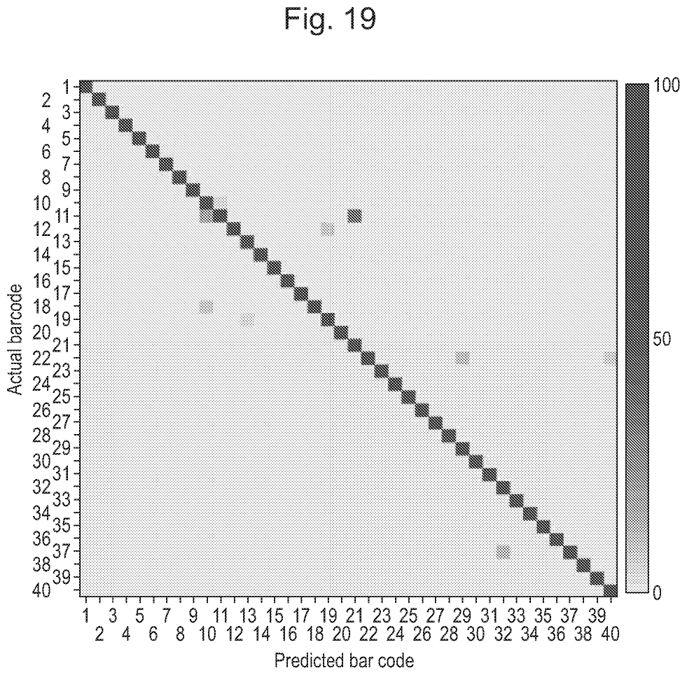

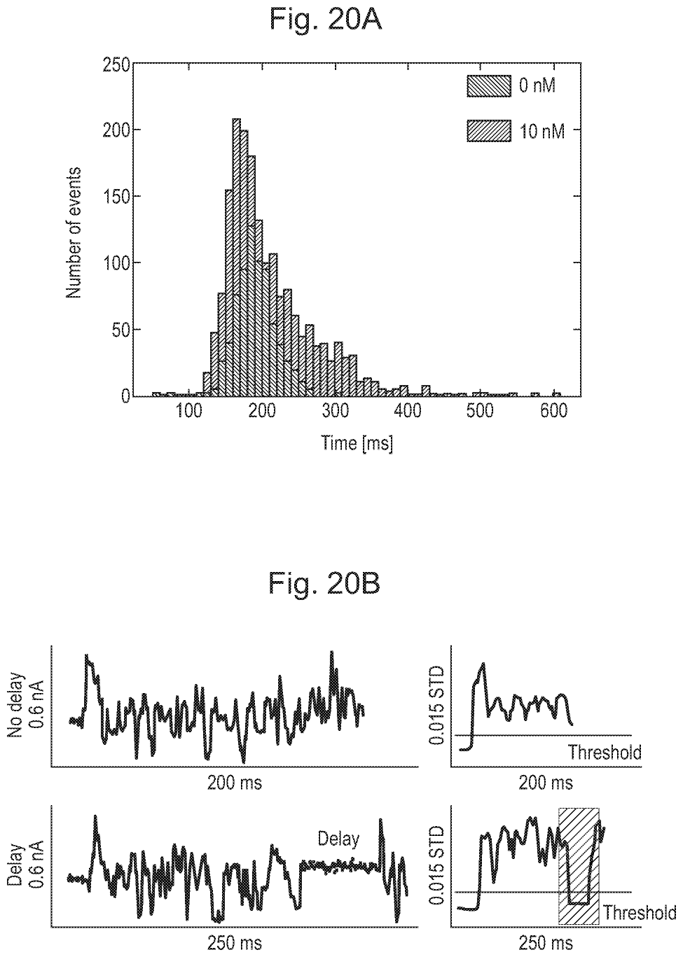

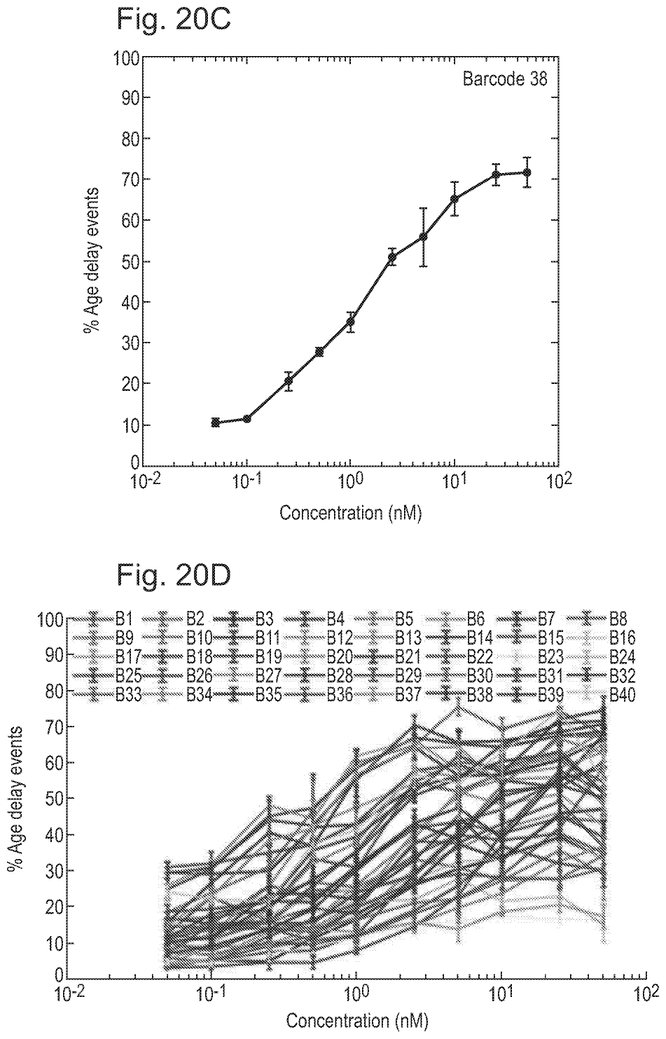

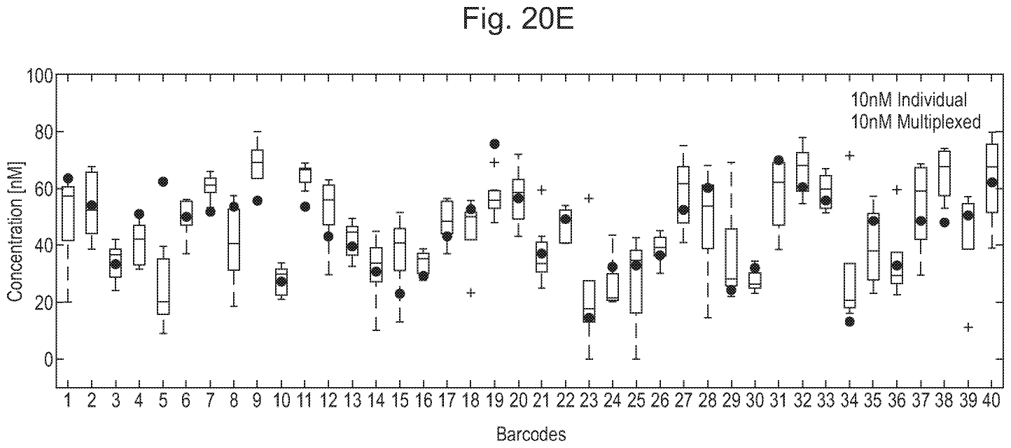

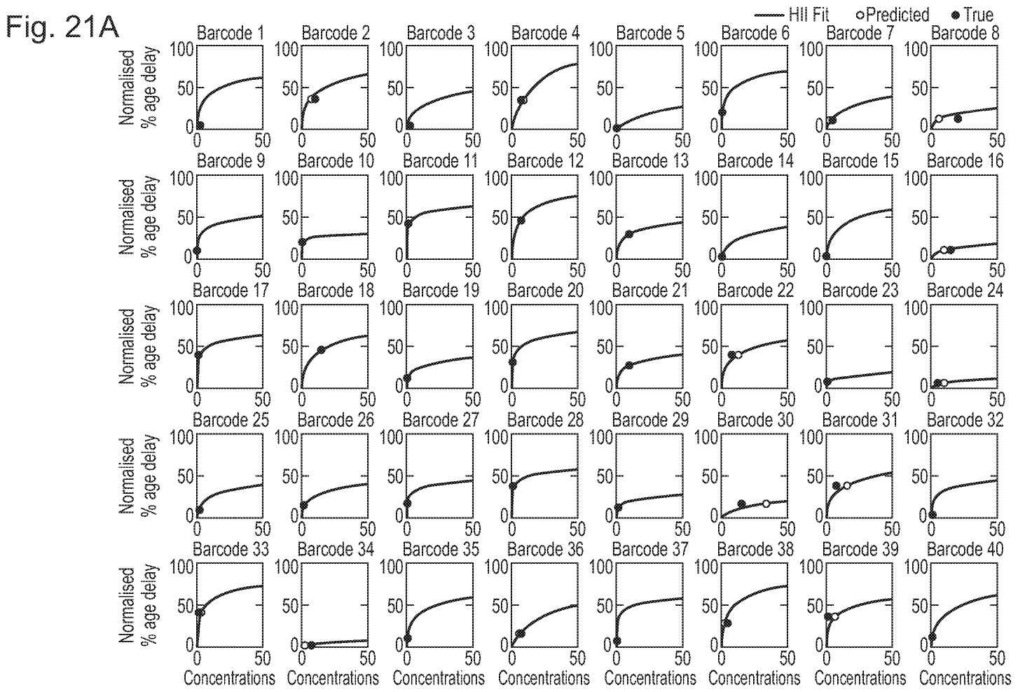

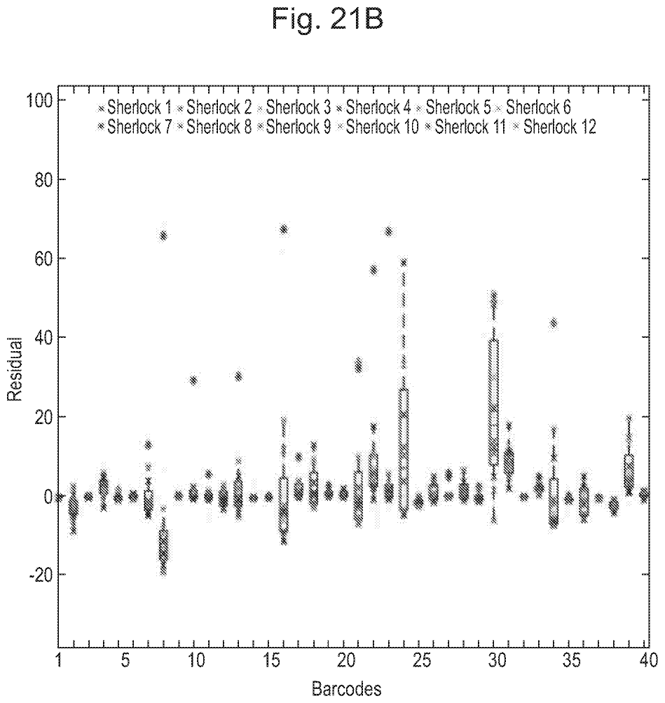

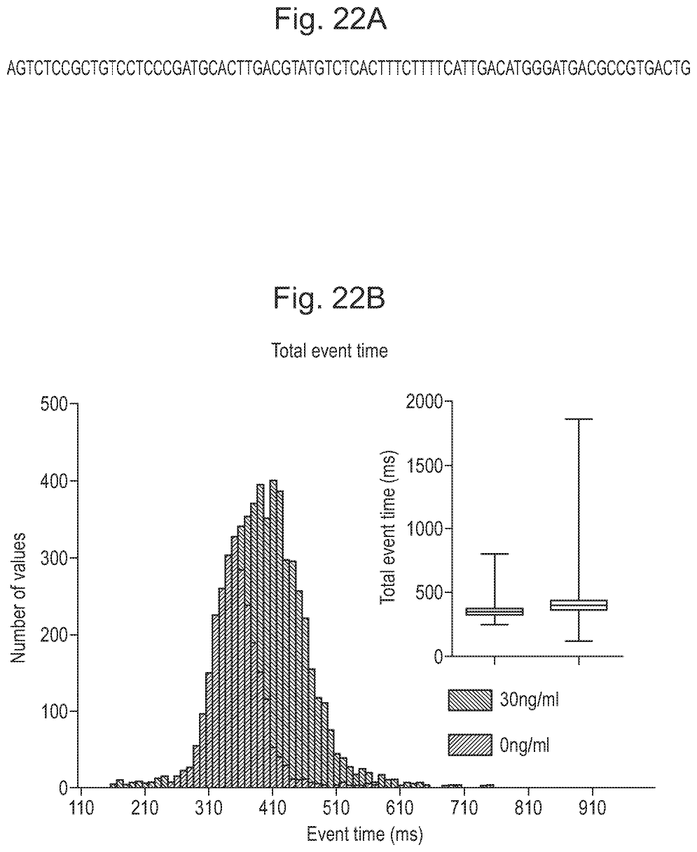

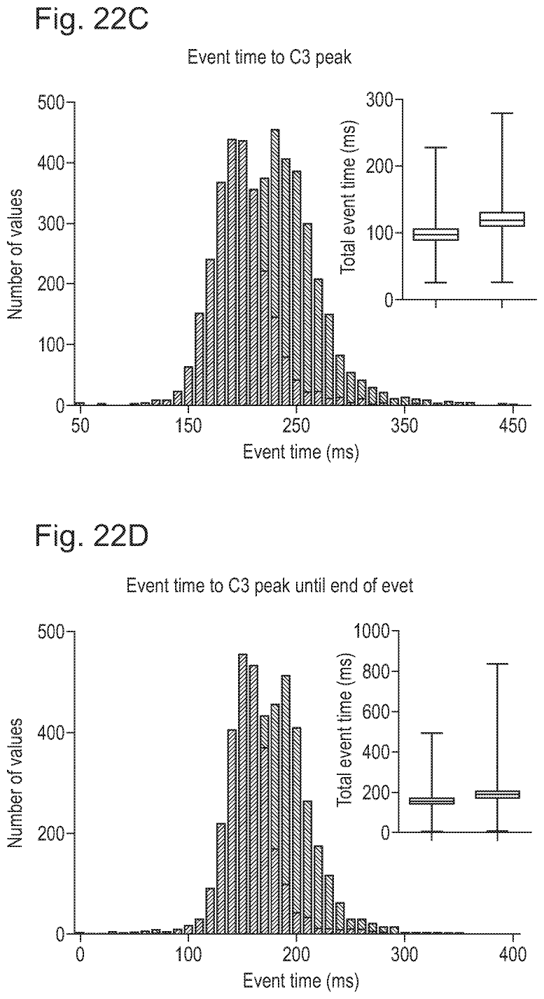

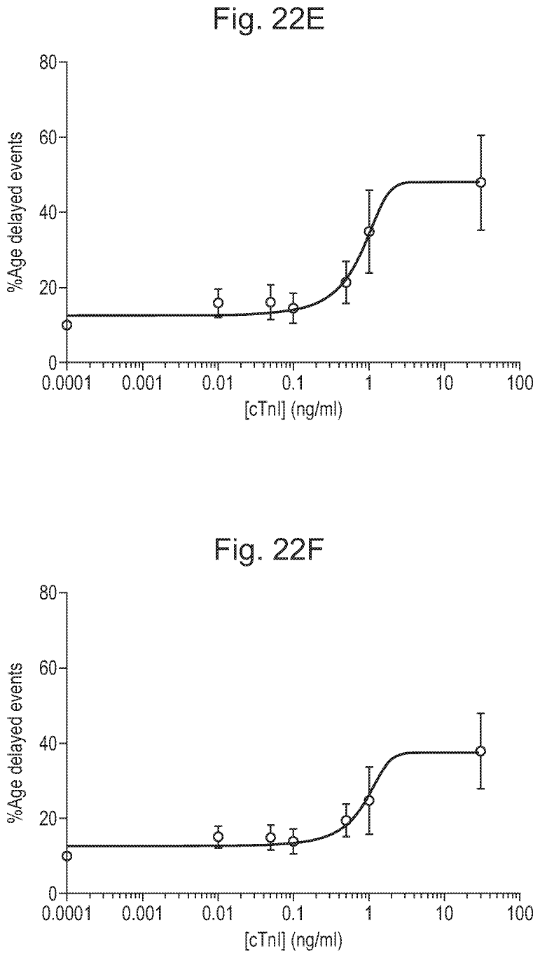

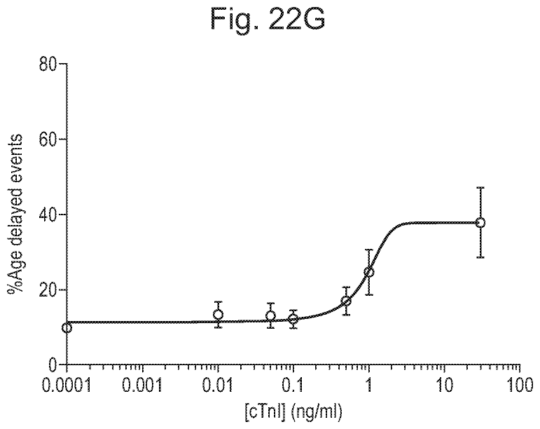

The barcode sequence had an accuracy of more than 90%. A false-positive rate of just 0.0001% was observed (FIG. 5(a)). The method had a very low preference for wrong barcode classification, as shown in the confusion matrix of FIG. 5(b). Improved algorithms and detection technologies will continue to improve barcode classification. Another way to improve barcode classification is include repeats of the barcode in the carrier as a proof-reading mechanism. The example shows that 10 barcoded carriers could be successfully discriminated within a complex mixture, allowing for highly multiplexed assays.

Identification of the Carriers that are Bound to an Analyte by Stalling Analysis.

In this example, stalling analysis was used to determine whether a target has bound to the barcoded strand or not. If the target analyte is not bound to the barcoded strand the current signal does not indicate stalling (in dwell time, current amplitude). Bound analytes (here an example is given with a complementary miRNA) stall the carrier which results an unique current profile (see FIG. 7). Stalling may be due, for example, to (1) unzipping of double stranded nucleotide structures (e.g. miRNA, DNA detection), (2) unravelling of either G-quadruplex or stem-loops aptamer structures (e.g. for the detection of protein, neurotransmitter and small bound molecular analytes), or (3) striping of bound antibody-antigens. The relative concentration of the target molecule can be determined by correlating the fraction of stalled vs non-stalled carriers (see FIG. 8). The absolute concentration of carriers in a sample can be determined by measuring time between individual single molecule detection events (inter event time) as widely described in nanopore literature.

Another way to determine the concentration of a molecule using the method described herein is to determine the concentration dependence of the molecule on the detection of carrier bound to molecule. For example, a standard curve may be prepared, in a similar manner to that shown in FIG. 9.

In another experiment, detection of binding between thrombin and a 15-mer thrombin binding aptamer was studied. Upon binding with thrombin, the “squiggle” events showed much longer dwell time, with significant increase in stalling found upon binding with 400 nM thrombin in terms of current flipping, corresponding to the unwinding of G-quadruplex and aptamer-protein interactions. Concentration dependence of the binding between thrombin and 15-mer thrombin binding aptamer was further verified by increasing thrombin concentration from 0 nM to 400 nM, and the increase of stalling was observed as more squiggle events with longer dwell times (see FIGS. 10 and 11).

In another experiment, detection of binding serotonin using the stem-loop aptamer was studied. The increase in stalling upon binding with serotonin was attributed to the structural reorganization of aptamer from loop to G-quadruplex upon binding with serotonin, resulting in the unwinding of loop, G-quadruplex and aptamer-serotonin interactions (see FIG. 12). Concentration dependence of the binding between serotonin and the aptamer was further verified by increasing serotonin concentration between 0 nM and 40 nM. The increase of stalling was observed as more squiggle events and longer dwell times (see FIG. 13).

Concentration dependence of the binding between serotonin and the aptamer was also observed based on average delay time vs concentration, as shown in Table 3, below.

TABLE 3

| Concentration (nM) | Average delay time (ms) |
|---|---|
| 0 | 95 |
| 2.5 | 97 |
| 5 | 103 |
| 10 | 112 |
| 20 | 135 |
| 40 | 144 |
| 60 | 187 |
| 80 | 388 |

In another experiment, detection of binding acetylcholine using a step-loop aptamer was studied. The barcode(underlined)-spacer-aptamer(italics) used was:

(SEQ ID NO: 48)
TCGATACAATACA
(SEQ ID NO: 175)
/spacer/ATCCGTCACACCTGCTCTAGGGGATCAAAGCT
ATGCGACCATGCGAGTGGATACTGGTGTTGGCTCCCGTAT

A clear stalling event can be seen upon binding with acetylcholine. Concentration dependence of the binding between acetylcholine and the aptamer on the delay (stalling) percentage was observed by increasing acetylcholine concentration between 0 nM and 40 nM (FIG. 14).

### Example 3

Alternative methods for identifying carriers that are bound to an analyte are available.

In some embodiments, it is not necessary to perform a specific stalling analysis. The basecalling software programs GUPPY and MinKNOW (Oxford Nanopore Technologies) may be used to directly analyse data generated as a carrier moves within a pore.

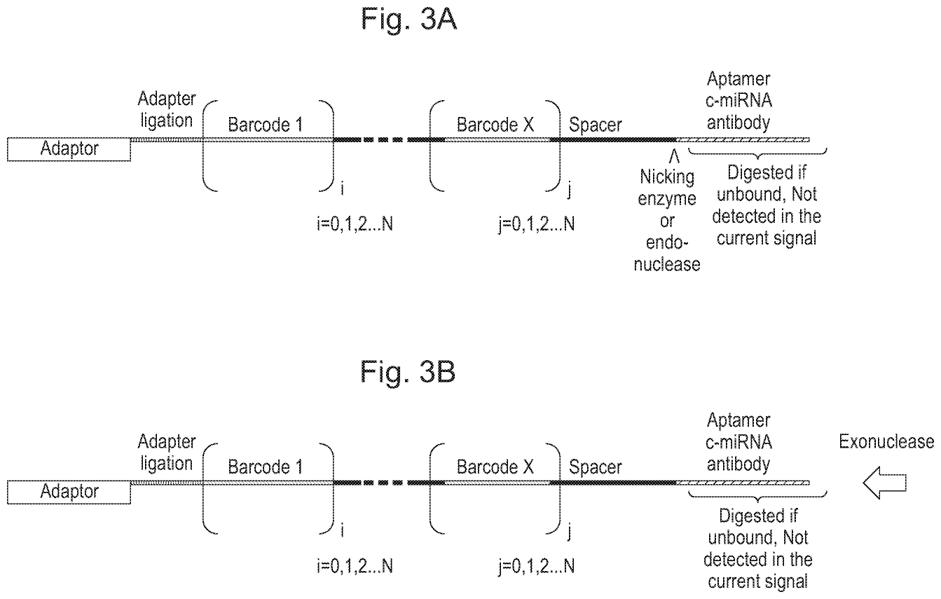

Separately, and as described above, another option is to use enzymatic digestion to remove/digest any part of carriers which have unbound molecule-binding region. This can be achieved by using a) endonucleases or b) exonuclease that target the molecule-binding region in carriers that are not bound to an analyte target (see FIG. 3). As such after digestion, the carriers that are bound to target and that are not bound to targets will be discriminated based the presence of current signal or the lack thereof after the barcode sequence. Alternatively, if the molecule-binding region is a polypeptide, proteases may be used that digest parts of carriers which have unbound molecule-binding region, provided that a difference in the signal may be observed when a carrier comprising an unbound and enzymatically-digested molecule-binding region passes within the pore, when compared to a carrier comprising a molecule bound to the molecule-binding region.

### Example 4

### Protocol for Assembly of Carriers

The barcodes (total concentration 30 nM, equal concentration for each) were incubated with ligation c-strand in a molar ratio of 1:3. The hybridisation was initiated by centrifuging at 4° C. for 1 minute, followed by incubating at room temperature for 1 h. The resultant mixture was mixed with 10 nM adapter, and the ligation was performed by adding an equal volume of TA ligase master mix (New England Biolabs). The sample was centrifuged at 4° C. for 1 minute to thoroughly mix different components while preserving the ligase at low temperature. After incubating at room temperature for 20 minutes, 1.4 times of the total volume of Ampure XP beads (Beckmann Coulter) was added to absorb nucleic acids for further purification. The beads were washed twice with short fragment buffer (Oxford Nanopore Technologies) to selectively remove excess amount of unhybridised ligation c-strands and barcodes that were not ligated. After purification, the beads were washed with nuclease-free water to wash off purified nucleic acids containing ligated motor protein-barcode complexes. The final solution of sequencing experiments was made up by the eluent, sequencing buffer, tether (100 nM), nuclease-free water, and incubated for at least 30 minutes with certain concentration of targeted analytes. For miRNA experiments, concentrations of 0.05 nM, 0.1 nM, 0.25 nM, 0.5 nM, 1 nM, 2.5 nM, 5 nM, 10 nM, 25 nM & 50 nM were used. For protein experiments, concentrations of 10 pg/mL, 50 pg/mL, 100 pg/mL, 500 pg/mL, 1 ng/mL, 30 ng/mL were used.

### Protocol for Experimental Run

All experiments were run for 30 minutes using the research and/or customer script at 37° C.

### Data Analysis Workflow

All experiments were analysed with the Nanopore App, a pre-existing custom-written MATLAB code. FAST5 sequencing files output from the nanopore sequencing device (MinION device; Oxford Nanopore Technologies) were uploaded into the app, where they were further processed. The app allows all 512 channels of the MinION to be uploaded and analysed individually or in bulk.

### Event Detection

Translocation events are detected by using a thresholding algorithm. First, a linear baseline is set between −0.16 and −2 nA, depending on the experiment. A step offset of 1.8 is used to define the start of the events. If the events cross the threshold of std 30, they are detected by the algorithm. Those events are further analysed by filtering for events larger than 0.1 s. All events for this time frame are defined as events.

Sequencing and Basecalling GUPPY and/or MinKNOW software (Oxford Nanopore Technologies) was used to sequence and basecall the events detected in the previous step.

### Alignment

After basecalling the raw read signals, the sequenced events were aligned to the reference sequences, in this case barcode sequences 1-40. The algorithm assigned a barcode to the event of the highest alignment score, if the following points were true:
1) sequence has to start with ‘GGG’,
2) at least 15 bases need to be aligned,
3) Only 1 mismatch in first 10 bases,
4) Only 1 mismatches in all aligned bases.

Only when all criteria were fulfilled, the max alignment was classified as barcode. If not, the event was removed and not considered for further analysis.

### Stalling

For the stalling analysis, the C3 parts, as well as the start and end of the event were defined. This was used to calculate the mean translocation time of all sequenced reads. If the translocation time of an event was significantly longer (typically used moving std 75 bins) compared to the mean, the event was classified as stalled. Moreover, each stalling needed to be greater than 10 bins to be considered as stalling.

### Results: Heatmap of 40 Barcodes (FIG. 19)

FIG. 19 presents a Confusion Matrix demonstrating a very low preference for incorrect barcode classification. All 40 barcodes tested were called with an accuracy of >95%.

Results: Detection of Multiple miRNAs

The multiplexed barcode sequencing method described herein enabled detection of 40 different miRNAs. Results are presented in FIG. 20.

Results: Quantification of Unknown miRNA Concentrations

The method described herein enabled accurate prediction of miRNA concentration, using blind testing of multiple different samples of known miRNA concentrations. Results are presented in FIG. 21.

Results: Detection of cTnI

Data relating to detection of the protein cardiac troponin I (cTnI) are shown in FIG. 22. The troponin aptamer sequence shown is as follows:

(SEQ ID NO: 49)
AGTCTCCGCTGTCCTCCCGATGCACTTGACGTATGTCTCA
CTTTCCTTTTCATTGAATGGGATGACGCCGTGACTG

### Annex to Example 4

Sequences of Carrier Strands—miRNAs (FIG. 17)

Adapter_barcode12_c-hsa-miR-27b-5p
(SEQ ID NO: 50)
/5Phos/ CCTAGTTCCGCTGGG ATCGCTACGCCTTCGGCTCGTAATCATAGTCGAGT
(SEQ ID NO: 176)
/iSpC3//iSpC3/GTTCACCAATCAGCTAAGCTCT
Adapter_barcode13_c-hsa-miR-21-5p
(SEQ ID NO: 51)
/5Phos/CCTAGTTCCGCTGGG AGCTCAGAGCAGGTCACTCAAGATACGAGCTGCGT
(SEQ ID NO: 177)
/iSpC3//iSpC3/TCAACATCAGTCTGATAAGCTA
Adapter_barcode14_c-hsa-miR-221-5p
(SEQ ID NO: 52)
/5Phos/CCTAGTTCCGCTGGG GTAAGTCTGCATCAGCGCGCGGCTGTGCGAGGATA
(SEQ ID NO: 178)
/iSpC3//iSpC3/AAATCTACATTGTATGCCAGGT
Adapter_barcode15_c-hsa-miR-30d-5p
(SEQ ID NO: 53)
/5Phos/CCTAGTTCCGCTGGG CTACGACAGTACGCTAGCAAGGATAGACACTACGA
(SEQ ID NO: 179)
/iSpC3//iSpC3/CTTCCAGTCGGGGATGTTTACA
Adapter_barcode16_c-hsa-miR-30c-5p
(SEQ ID NO: 54)
/5Phos/CCTAGTTCCGCTGGG TACTGAACACAAGTTCGTCGTCGAGCAATCACAAT
(SEQ ID NO: 180)
/iSpC3//iSpC3/GCTGAGAGTGTAGGATGTTTACA
Adapter_barcode19_c-hsa-miR-181b-5p
(SEQ ID NO: 55)
/5Phos/CCTAGTTCCGCTGGG CTAGTGCGCAGTIGTCTCGGCGGAGTTGAGACTGA
(SEQ ID NO: 181)
/iSpC3//iSpC3/ACCCACCGACAGCAATGAATGTT
Adapter_barcode20_c-hsa-miR-29a-3p
(SEQ ID NO: 56)
/5Phos/CCTAGTTCCGCTGGG GATCATGGTAGTCTTCAAGATCGAGTATGTCTGTC
(SEQ ID NO: 182)
/iSpC3//iSpC3/TAACCGATTTCAGATGGTGCTA
Adapter_barcode22_c-hsa-miR-210-5p
(SEQ ID NO: 57)
/5Phos/CCTAGTTCCGCTGGG GTTCACATCAAGGTCATACCGCGAGTTCTATTTTA
(SEQ ID NO: 183)
/iSpC3//iSpC3/CAGTGTGCGGTGGGCAGGGGCT
Adapter_barcode24_c-hsa-miR-126a-5p
(SEQ ID NO: 58)
/5Phos/CCTAGTTCCGCTGGG GCTTGGGGGATAGATGTGCCCCGCGCATCGGACCT
(SEQ ID NO: 184)
/iSpC3//iSpC3/CGCGTACCAAAAGTAATAATG
Adapter_barcode25_c-hsa-mir-1306-5p
(SEQ ID NO: 59)
/5Phos/CCTAGTTCCGCTGGG ATGACACACGTTTTCGATAGGGACGCCGACTTTAA
(SEQ ID NO: 185)
/iSpC3//iSpC3/TGGACGTTTGCAGGGGAGGTGG
Adapter_barcode26_c-hsa-miR-126-5p
(SEQ ID NO: 60)
/5Phos/CCTAGTTCCGCTGGG TGATAATAAGACCTGACAGACAATAGGGAGAACTC
(SEQ ID NO: 186)
/iSpC3//iSpC3/CGCGTACCAAAAGTAATAATG
Adapter_barcode27_c-hsa-miR-1254
(SEQ ID NO: 61)
/5Phos/CCTAGTTCCGCTGGG TTAGTAATCAAGTCTGATCGTAATAGCTAAGTCAT
/iSpC3//iSpC3/ACTGCAGGCTCCAGCTTCCAGGCT
(SEQ ID NO: 187)
Adapter_barcode29_c-hsa-miR-30e-5p
(SEQ ID NO: 62)
/5Phos/CCTAGTTCCGCTGGG CCTGTTCTAATTGCGCGGAGAGGCGAGATGTTTCT
(SEQ ID NO: 188)
/iSpC3//iSpC3/CTTCCAGTCAAGGATGTTTACA
Adapter_barcode30_c-hsa-miR-106a-5p
(SEQ ID NO: 63)
/5Phos/CCTAGTTCCGCTGGG TGGGATACTGACGTGCCGGAATACCCAGACGTGCC
(SEQ ID NO: 189)
/iSpC3//iSpC3/CTACCTGCACTGTAAGCACTTTT
Adapter_barcode31_c-hsa-miR-199a-3p
(SEQ ID NO: 64)
/5Phos/CCTAGTTCCGCTGGG TGATCGGCACCTAAACGCATTAGCCCTGCAATACG
(SEQ ID NO: 190)
/iSpC3//iSpC3/TAACCAATGTGCAGACTACTGT
Adapter_barcode32_c-hsa-miR-652-3p
(SEQ ID NO: 65)
/5Phos/CCTAGTTCCGCTGGG AGCTGCTCGGAAGCCATAAGGTACTTTAATTTGGG
(SEQ ID NO: 191)
/iSpC3//iSpC3/CACAACCCTAGTGGCGCCATT
Adapter_barcode33_c-hsa-miR-26b-5p
(SEQ ID NO: 66)
/5Phos/CCTAGTTCCGCTGGG CACGGATTTCTATATTGCTCAACCAGGCAGCGCAA
(SEQ ID NO: 192)
/iSpC3//iSpC3/ACCTATCCTGAATTACTTGAA
Adapter_barcode34_c-hsa-miR-145-5p
(SEQ ID NO: 67)
/5Phos/CCTAGTTCCGCTGGG ATTGTGCGCTTTTCTCCATGCGTCTTAGACATCTC
(SEQ ID NO: 193)
/iSpC3//iSpC3/AGGGATTCCTGGGAAAACTGGAC
Adapter_barcode35_c-hsa-miR-92a-3p
(SEQ ID NO: 68)
/5Phos/CCTAGTTCCGCTGGG GTGAAATCCCCGTCTAGGTTATGGCTGGGGGGATT
(SEQ ID NO: 194)
/iSpC3//iSpC3/ACAGGCCGGGACAAGTGCAATA
Adapter_barcode36_c-hsa-miR-146a-5p
(SEQ ID NO: 69)
/5Phos/CCTAGTTCCGCTGGG CGTAAACTTATCACGACACAATGAACAAGCCTGCA
(SEQ ID NO: 195)
/iSpC3//iSpC3/AACCCATGGAATTCAGTTCTCA
Adapter_barcode37_c-hsa-miR-423-5p
(SEQ ID NO: 70)
/5Phos/CCTAGTTCCGCTGGG GGCAGTGTCCGAGCGTCCTCAATCATGAGCGATTC
(SEQ ID NO: 196)
/iSpC3//iSpC3/AAAGTCTCGCTCTCTGCCCCTCA
Adapter_barcode39_c-hsa-miR-27b-3p
(SEQ ID NO: 71)
/5Phos/CCTAGTTCCGCTGGG CGTCCAGACTTAATGTCTGCTCACTGACATCGCGA
(SEQ ID NO: 197)
/iSpC3//iSpC3/GCAGAACTTAGCCACTGTGAA
Adapter_barcode40_c-hsa-miR-1-3p
(SEQ ID NO: 72)
/5Phos/CCTAGTTCCGCTGGG ATTAGCGGAACCAAACCCAGGAAGGCTTGAAGGCG
(SEQ ID NO: 198)
/iSpC3//iSpC3/ATACATACTTCTTTACATTCCA
Adapter_barcode42_c-hsa-miR-18a-5p
(SEQ ID NO: 73)
/5Phos/CCTAGTTCCGCTGGG AACCTTAGGGGCCTCGAATCTTTGAGACGACTAGG
(SEQ ID NO: 199)
/iSpC3//iSpC3/CTATCTGCACTAGATGCACCTTA
Adapter_barcode43_c-hsa-miR-18b-5p
(SEQ ID NO: 74)
/5Phos/CCTAGTTCCGCTGGG TAATTACTGCCCCACCATGACATTTTAATAGCAGT
(SEQ ID NO: 200)
/iSpC3//iSpC3/CTAACTGCACTAGATGCACCTTA
Adapter_barcode45_c-hsa-miR-301a-5p
(SEQ ID NO: 75)
/5Phos/CCTAGTTCCGCTGGG GACCTTGAGACAGAACTTATCAATGTACAACTGAA
(SEQ ID NO: 201)
/iSpC3//iSpC3/AGTAGTGCAATAAAGTCAGAGC
Adapter_barcode46_c-hsa-let7c-5p
(SEQ ID NO: 76)
/5Phos/CCTAGTTCCGCTGGG CGAAGGATTGGCCCCCCGATTACCACCGCCGTGAG
(SEQ ID NO: 202)
/iSpC3//iSpC3/AACCATACAACCTACTACCTCA
Adapter_barcode47_c-hsa-miR-125a-5p
(SEQ ID NO: 77)
/5Phos/CCTAGTTCCGCTGGG AATTGCCAACAGGTCAAGCCCTGTTCTCACTGGTC
(SEQ ID NO: 203)
/iSpC3//iSpC3/TCACAGGTTAAAGGGTCTCAGGGA
Adapter_barcode49_c-hsa-miR-190a-3p
(SEQ ID NO: 78)
/5Phos/CCTAGTTCCGCTGGG CAGTGCTTGCCCCCAGTAGAGTGTGGAAGGGCATA
(SEQ ID NO: 204)
/iSpC3//iSpC3/AGGAATATGTTTGATATATAG
Adapter_barcode50_c-hsa-miR-193b-3p
(SEQ ID NO: 79)
/5Phos/CCTAGTTCCGCTGGG AAAGCCCACTCTCCACACTTCAAGGTTAAATGGCG
(SEQ ID NO: 205)
/iSpC3//iSpC3/AGCGGGACTTTGAGGGCCAGTT
Adapter_barcode51_c-hsa-miR-193a-5p
(SEQ ID NO: 80)
/5Phos/CCTAGTTCCGCTGGG GACTAATACAATCGGAAGCAACTCTCACGCCGCAC
(SEQ ID NO: 206)
/iSpC3//iSpC3/TCATCTCGCCCGCAAAGACCCA
Adapter_barcode52_c-hsa-miR-211-5p
(SEQ ID NO: 81)
/5Phos/CCTAGTTCCGCTGGG CTACGATTCATGTCTCCCCCCACATATGATTGATC
(SEQ ID NO: 207)
/iSpC3//iSpC3/AGGCGAAGGATGACAAAGGGAA
Adapter_barcode53_c-hsa-miR-545-5p
(SEQ ID NO: 82)
/5Phos/CCTAGTTCCGCTGGG TCGGGCGCTTAATCGCAATGTTCATCCGGAACGGA
(SEQ ID NO: 208)
/iSpC3//iSpC3/TCATCTAATAAACATTTACTGA
Adapter_barcode54_c-hsa-miR-550a-5p
(SEQ ID NO: 83)
/5Phos/CCTAGTTCCGCTGGG TAAATTCCACATTACGATAGCTGACGTCCTGGTAG
(SEQ ID NO: 209)
/iSpC3//iSpC3/GGGCTCTTACTCCCTCAGGCACT
Adapter_barcode55_c-hsa-miR-638
(SEQ ID NO: 84)
/5Phos/CCTAGTTCCGCTGGG CACCCCCTAGCCTGACCTTAAATGATAATTCCTGT
(SEQ ID NO: 210)
/iSpC3//iSpC3/AGGCCGCCACCCGCCCGCGATCCCT
Adapter_barcode56_c-hsa-miR-671-5p
(SEQ ID NO: 85)
/5Phos/CCTAGTTCCGCTGGG TTTAGCCTAACCTCTGATGCATTGCACCGGAGTTC
(SEQ ID NO: 211)
/iSpC3//iSpC3/CTCCAGCCCCTCCAGGGCTTCCT
Adapter_barcode57_c-hsa-miR-1233-5p
(SEQ ID NO: 86)
/5Phos/CCTAGTTCCGCTGGG CCCAATATACGCTGAACCTTCCATCCGATTTTCAG
(SEQ ID NO: 212)
/iSpC3//iSpC3/TGCCGTGCCCTGGCCTCCCACT
Adapter_barcode58_c-hsa-miR-3135b
(SEQ ID NO: 87)
/5Phos/CCTAGTTCCGCTGGG CCTTGGAATTAGAACCGTGTGATTCTACGCCTAGG
(SEQ ID NO: 213)
/iSpC3//iSpC3/CACCACTGCACTCGCTCCAGCC
Adapter_barcode59_c-hsa-miR-3908
(SEQ ID NO: 88)
/5Phos/CCTAGTTCCGCTGGG AGGACTTCTTCGGTGTAATCGGAGTACATCAATGT
(SEQ ID NO: 214)
/iSpC3//iSpC3/AAACAGTCTACCTACATTGCTC
Adapter_barcode60_c-hsa-miR-5571-5p
(SEQ ID NO: 89)
/5Phos/CCTAGTTCCGCTGGG AACGTACTTTGTGGGGATAAGCTGTACAGGGCTGT
(SEQ ID NO: 215)
/iSpC3//iSpC3/GGGAGGCTCCTTTGAGAATTG

### Sequences of Carrier Strands—Protein & Neurotransmitter (FIG. 18)

Cardiac Troponin I (cTnI):

Barcode 65
(SEQ ID NO: 90)
/5Phos/CCTAGTTCCGCTGGG ATTCAGATCTTTGACCGATCGTACTGACGTGTACG
(SEQ ID NO: 216)
/iSpC3//iSpC3/AGTCTCCGCTGTCCTCCCGATGCACTTGACGTATGTCTCACTTT
CTTTTCATTGACATGGGATGACGCCGTGACTG
Cardiac troponin T (cTnT):
Barcode 62
(SEQ ID NO: 91)
/5Phos/CCTAGTTCCGCTGGG CTTAAGTTCGGTCATTAACAGTGTCAATCTTGCCA
(SEQ ID NO: 217)
/iSpC3//iSpC3/ATACGGGAGCCAACACCAGGACTAACATTATAAGAATTGCGAATA
ATCATTGGAGAGCAGGTGTGACGGAT
BNP:
Barcode 61
(SEQ ID NO: 92)
/5Phos/CCTAGTTCCGCTGGG ATGTAATTCCTGCTAGGTCCAATGTGACTGTACTA
(SEQ ID NO: 218)
/iSpC3//iSpC3/GGCGATTCGTGATCTCTGCTCTCGGTTTCGCGTTCGTTCG
Thrombin:
Barcode 70
(SEQ ID NO: 93)
/5Phos/CCTAGTTCCGCTGGG TTAAGCTATTGCTAACTGTAGTCCTAGTCTAGCTA
(SEQ ID NO: 219)
/iSpC3//iSpC3/AGTCCGTGGTAGGGCAGGTTGGGGTGACT
Barcode 1
(SEQ ID NO: 94)
/5Phos/CCTAGTTCCGCTGGG GAGCTAGCATACGTGTCTAACACTGCACAGATGAT
(SEQ ID NO: 220)
/iSpC3//iSpC3/ GGTTGGTGTGGTTGG
S-protein:
Barcode 7
(SEQ ID NO:95)
/5Phos/CCTAGTTCCGCTGGG ACCTTTTCTAGGATGGAACATTCTA
(SEQ ID NO: 221)
/iSpC3//iSpC3/CACGCATAACGTCTTGCGGGGCGGCGGGTTGAGAGGATGTGGGG
TGGTTATGCGTG
N-protein:
Barcode 6
(SEQ ID NO:96)
/5Phos/CCTAGTTCCGCTGGG CTGCGACATGACTATCTAGAGTCGC
(SEQ ID NO: 222)
/iSpC3/iSpC3/GCTGGATGTCACCAGATTGTCGGACATCGGATTGTCTGAGTCAT
ATGACACATCCAGC
Serotonin:
Barcode 5
(SEQ ID NO: 97)
/5Phos/CCTAGTTCCGCTGGG ACTAGATAAAAGGAAGGGAGCACAGTAACGTCGTT
(SEQ ID NO: 223)
/iSpC3/iSpC3/CGACTGGTAGGCAGATAGGGGAAGCTGATTCGATG
Acetylcholine:
Barcode 4
(SEQ ID NO: 98)
/5Phos/CCTAGTTCCGCTGGG TCGATACAATACA
(SEQ ID NO: 224)
/iSpC3/iSpC3/ATCCGTCACACCTGCTCTAGGGGATCAAAGCTATGCGACCATGC
GAGTGGATACTGGTGTTGGCTCCCGTAT

### Target Analytes

miRNAs:

| | | |
|---|---|---|
| 11 | hsa-miR-497-5p | rCrArGrCrArGrCrArCrArCrUrGrUrGrGrUrUrUrGrU |
| | | (SEQ ID NO: 99) |
| 12 | hsa-miR-27b-5p | rArGrArGrCrUrUrArGrCrUrGrArUrUrGrGrUrGrArArC |
| | | (SEQ ID NO: 100) |
| 13 | hsa-miR-21-5p | rUrArGrCrUrUrArUrCrArGrArCrUrGrArUrGrUrUrGrA |
| | | (SEQ ID NO: 101) |
| 14 | hsa-miR-221-5p | rArCrCrUrGrGrCrArUrArCrArArUrGrUrArGrArUrUrU |
| | | (SEQ ID NO: 102) |
| 15 | hsa-miR-30d-5p | rUrGrUrArArArCrArUrCrCrCrCrGrArCrUrGrGrArArG |
| | | (SEQ ID NO: 103) |
| 16 | hsa-miR-30c-5p | rUrGrUrArArArCrArUrCrCrUrArCrArCrUrCrUrCrArGrC |
| | | (SEQ ID NO: 104) |
| 17 | hsa-miR-133a-5p | rArGrCrUrGrGrUrArArArArUrGrGrArArCrCrArArArU |
| | | (SEQ ID NO: 105) |
| 18 | hsa-miR-208a-5p | rGrArGrCrUrUrUrUrGrGrCrCrCrGrGrGrUrUrArUrArC |
| | | (SEQ ID NO: 106) |
| 19 | hsa-miR-181b-5p | rArArCrArUrUrCrArUrUrGrCrUrGrUrCrGrGrUrGrGrGrU |
| | | (SEQ ID NO: 107) |
| 20 | hsa-miR-29a-3p | rUrArGrCrArCrCrArUrCrUrGrArArArUrCrGrGrUrUrA |
| | | (SEQ ID NO: 108) |
| 22 | hsa-miR-210-5p | rArGrCrCrCrCrUrGrCrCrCrArCrCrGrCrArCrArCrUrG |
| | | (SEQ ID NO: 109) |
| 24 | hsa-miR-126a-5p | rCrArUrUrArUrUrArCrUrUrUrUrGrGrUrArCrGrCrG |
| | | (SEQ ID NO: 110) |
| 25 | hsa-mir-1306-5p | rCrCrArCrCrUrCrCrCrCrUrGrCrArArArCrGrUrCrCrA |
| | | (SEQ ID NO: 111) |
| 26 | hsa-miR-126-5p | rCrArUrUrArUrUrArCrUrUrUrUrGrGrUrArCrGrCrG |
| | | (SEQ ID NO: 112) |
| 27 | hsa-miR-1254 | rArGrCrCrUrGrGrArArGrCrUrGrGrArGrCrCrUrGrCrArGrU |
| | | (SEQ ID NO: 113) |
| 29 | hsa-miR-30e-5p | rUrGrUrArArArCrArUrCrCrUrUrGrArCrUrGrGrArArG |
| | | (SEQ ID NO: 114) |
| 30 | hsa-miR-106a-5p | rArArArArGrUrGrCrUrUrArCrArGrUrGrCrArGrGrUrArG |
| | | (SEQ ID NO: 115) |
| 31 | hsa-miR-199a-3p | rArCrArGrUrArGrUrCrUrGrCrArCrArUrUrGrGrUrUrA |
| | | (SEQ ID NO: 116) |
| 32 | hsa-miR-652-3p | rArArUrGrGrCrGrCrCrArCrUrArGrGrGrUrUrGrUrG |
| | | (SEQ ID NO: 117) |
| 33 | hsa-miR-26b-5p | rUrUrCrArArGrUrArArUrUrCrArGrGrArUrArGrGrU |
| | | (SEQ ID NO: 118) |
| 34 | hsa-miR-145-5p | rGrUrCrCrArGrUrUrUrUrCrCrCrArGrGrArArUrCrCrCrU |
| | | (SEQ ID NO: 119) |
| 35 | hsa-miR-92a-3p | rUrArUrUrGrCrArCrUrUrGrUrCrCrCrGrGrCrCrUrGrU |
| | | (SEQ ID NO: 120) |
| 36 | hsa-miR-146a-5p | rUrGrArGrArArCrUrGrArArUrUrCrCrArUrGrGrGrUrU |
| | | (SEQ ID NO: 121) |
| 37 | hsa-miR-423-5p | rUrGrArGrGrGrGrCrArGrArGrArGrCrGrArGrArCrUrUrU |
| | | (SEQ ID NO: 122) |
| 39 | hsa-miR-27b-3p | rUrUrCrArCrArGrUrGrGrCrUrArArGrUrUrCrUrGrC |
| | | (SEQ ID NO: 123) |
| 40 | hsa-miR-1-3p | rUrGrGrArArUrGrUrArArArGrArArGrUrArUrGrUrArU |
| | | (SEQ ID NO: 124) |
| 42 | hsa-miR-18a-5p | rUrArArGrGrUrGrCrArUrCrUrArGrUrGrCrArGrArUrArG |
| | | (SEQ ID NO: 125) |
| 43 | hsa-miR-18b-5p | rUrArArGrGrUrGrCrArUrCrUrArGrUrGrCrArGrUrUrArG |
| | | (SEQ ID NO: 126) |
| 45 | hsa-miR-301a-5p | rGrCrUrCrUrGrArCrUrUrUrArUrUrGrCrArCrUrArCrU |
| | | (SEQ ID NO: 127) |
| 46 | hsa-let7c-5p | rUrGrArGrGrUrArGrUrArGrGrUrUrGrUrArUrGrGrUrU |
| | | (SEQ ID NO: 128) |
| 47 | hsa-miR-125a-5p | rUrCrCrCrUrGrArGrArCrCrCrUrUrUrArArCrCrUrGrUrGrA |
| | | (SEQ ID NO: 129) |
| 49 | hsa-miR-190a-3p | rCrUrArUrArUrArUrCrArArArCrArUrArUrUrCrCrU |
| | | (SEQ ID NO: 130) |
| 50 | hsa-miR-193b-3p | rArArCrUrGrGrCrCrCrUrCrArArArGrUrCrCrCrGrCrU |
| | | (SEQ ID NO: 131) |
| 51 | hsa-miR-193a-5p | rUrGrGrGrUrCrUrUrUrGrCrGrGrGrCrGrArGrArUrGrA |
| | | (SEQ ID NO: 132) |
| 52 | hsa-miR-211-5p | rUrUrCrCrCrUrUrUrGrUrCrArUrCrCrUrUrCrGrCrCrU |
| | | (SEQ ID NO: 133) |
| 53 | hsa-miR-545-5p | rUrCrArGrUrArArArUrGrUrUrUrArUrUrArGrArUrGrA |
| | | (SEQ ID NO: 134) |
| 54 | hsa-miR-550a-5p | rArGrUrGrCrCrUrGrArGrGrGrArGrUrArArGrArGrCrCrC |
| | | (SEQ ID NO: 135) |
| 55 | hsa-miR-638 | rArGrGrGrArUrCrGrCrGrGrGrCrGrGrGrUrGrGrCrGrGrCrCrU |
| | | (SEQ ID NO: 136) |
| 56 | hsa-miR-671-5p | rArGrGrArArGrCrCrCrUrGrGrArGrGrGrGrCrUrGrGrArG |
| | | (SEQ ID NO: 137) |
| 57 | hsa-miR-1233-5p | rArGrUrGrGrGrArGrGrCrCrArGrGrGrCrArCrGrGrCrA |
| | | (SEQ ID NO: 138) |
| 58 | hsa-miR-3135b | rGrGrCrUrGrGrArGrCrGrArGrUrGrCrArGrUrGrGrUrG |
| | | (SEQ ID NO: 139) |
| 59 | hsa-miR-3908 | rGrArGrCrArArUrGrUrArGrGrUrArGrArCrUrGrUrUrU |
| | | (SEQ ID NO: 140) |
| 60 | hsa-miR-5571-5p | rCrArArUrUrCrUrCrArArArGrGrArGrCrCrUrCrCrC |
| | | (SEQ ID NO: 141) |

### Proteins:

Cardiac troponin I (cTnI) [Genscript]
Cardiac Troponin T (cTnT): TNNT2 Protein Human Recombinant|CTnT Antigen|
ProSpec (prospecbio.com)
BNP-32 peptide: [Bachem]
Human alpha-Thrombin Native protein, Biotin (RP-43103) [Thermofisher]

### Reference Example 1

Limitations of multiplex assays that do not use a motor protein.

As described above, the presence of a motor protein on the carrier allows for accurate identification of the identifier region and the determination of the presence or absence of a molecule on the molecule-binding region. When the methods are carried out in the absence of a motor protein, there are limitations placed on the measurements that can be obtained.

In one experiment, increasing concentrations of thrombin were detected by nanopore measurements using a carrier comprising an aptamer (underlined) and a 30*T threading strand:

(SEQ ID NO: 142)
TTTTTTTTTTTTTTTTTTTTTTTTTTTTTTAGTCCG
TGGTAGGGCAGGTTGGGGTGACT (G4)

With this approach, it is possible to quantify the concentration of thrombin but there is no way of confirming false positives, nor is there an easy way to multiplex the assay. All that is measured is the difference in the signal level between aptamer and protein-bound aptamer (FIG. 15A).

In a second experiment, increasing concentrations of serotonin were detected by nanopore measurement using a carrier comprising a stem-loop aptamer (underlined) and a 30*T threading strand:

(SEQ ID NO: 143)
TTTTTTTTTTTTTTTTTTTTTTTTTTTTTTCGACTGG
TAGGCAGATAGGGGAAGCTGATTCGATGCGTGGGTCG

In the same way as the thrombin experiment, it is possible to quantify the concentration of serotonin but there is no way of confirming false positives, nor is there an easy way to multiplex the assay. All that is measured is the difference in the signal level between aptamer and neurotransmitter-bound aptamer (FIG. 15B).

In a third experiment, the levels of miRNA were detected in a multiplex format. Barcodes 1 to 6 (ACGTA, GGACT, TTAAC, GCTAG, CTGAG and TAGCG) were identified by unique average currents observed for each barcode (122.4 pA, 121.8 pA, 111.5 pA, 152.7 pA, 129.3 pA, 135.4 pA, respectively)(FIG. 14C). However, this approach is limited in its multiplexing ability and limited by the amplitude fluctuation in the signal. Realistically, five barcodes could be used in a multiplex assay. The assignment and classification of the barcodes would not necessarily be accurately classified due to the distributions in amplitudes observed for each barcode which overlap with the characteristic signals of other barcodes.

### SEQUENCE LISTING

SEQUENCE LISTING
exonuclease I from E. coli
SEQ ID NO: 1
MMNDGKQQSTFLFHDYETFGTHPALDRPAQFAAIRTDSEFNVIGE
PEVFYCKPADDYLPQPGAVLITGITPQEARAKGENEAAFAARIHS
LFTVPKTCILGYNNVREDDEVTRNIFYRNFYDPYAWSWQHDNSRW
DLLDVMRACYALRPEGINWPENDDGLPSFRLEHLTKANGIEHSNA
HDAMADVYATIAMAKLVKTRQPRLFDYLFTHRNKHKLMALIDVPQ
MKPLVHVSGMFGAWRGNTSWVAPLAWHPENRNAVIMVDLAGDIS
PLLELDSDTLRERLYTAKTDLGDNAAVPVKLVHINKCPVLAQANT
LRPEDADRLGINRQHCLDNLKILRENPQVREKVVAIFAEAEPFTP
SDNVDAQLYNGFFSDADRAAMKIVLETEPRNLPALDITFVDKRIE
KLLFNYRARNFPGTLDYAEQQRWLEHRRQVFTPEFLQGYADELQM
LVQQYADDKEKVALLKALWQYAEEIVSGSGHHHHHH
exonuclease III enzyme from E. coli
SEQ ID NO: 2
MKFVSFNINGLRARPHQLEAIVEKHQPDVIGLQETKVHDDMFPLE
EVAKLGYNVFYHGQKGHYGVALLTKETPIAVRRGFPGDDEEAQRR
IIMAEIPSLLGNVTVINGYFPQGESRDHPIKFPAKAQFYQNLQNY
LETELKRDNPVLIMGDMNISPTDLDIGIGEENRKRWLRTGKCSFL
PEEREWMDRLMSWGLVDTFRHANPQTADRFSWFDYRSKGFDDNRG
LRIDLLLASQPLAECCVETGIDYEIRSMEKPSDHAPVWATFRR
RecJ enzyme from T. thermophilus
SEQ ID NO: 3
MFRRKEDLDPPLALLPLKGLREAAALLEEALRQGKRIRVHGDYDA
DGLTGTAILVRGLAALGADVHPFIPHRLEEGYGVLMERVPEHLEA
SDLFLTVDCGITNHAELRELLENGVEVIVTDHHTPGKTPPPGLVV
HPALTPDLKEKPTGAGVAFLLLWALHERLGLPPPLEYADLAAVGT
IADVAPLWGWNRALVKEGLARIPASSWVGLRLLAEAVGYTGKAVE
VAFRIAPRINAASRLGEAEKALRLLLTDDAAEAQALVGELHRLNA
RRQTLEEAMLRKLLPQADPEAKAIVLLDPEGHPGVMGIVASRILE
ATLRPVFLVAQGKGTVRSLAPISAVEALRSAEDLLLRYGGHKEAA
GFAMDEALFPAFKARVEAYAARFPDPVREVALLDLLPEPGLLPQV
FRELALLEPYGEGNPEPLFL
bacteriophage lambda exonuclease
SEQ ID NO: 4
MTPDIILQRTGIDVRAVEQGDDAWHKLRLGVITASEVHNVIAKPR
SGKKWPDMKMSYFHTLLAEVCTGVAPEVNAKALAWGKQYENDART
LFEFTSGVNVTESPIIYRDESMRTACSPDGLCSDGNGLELKCPFT
SRDFMKFRLGGFEAIKSAYMAQVQYSMWVTRKNAWYFANYDPRMK
REGLHYVVIERDEKYMASFDEIVPEFIEKMDEALAEIGFVFGEQW
R
Phi29 DNA polymerase
SEQ ID NO: 5
MKHMPRKMYSCAFETTTKVEDCRVWAYGYMNIEDHSEYKIGNSLD
EFMAWVLKVQADLYFHNLKFDGAFIINWLERNGFKWSADGLPNTY
NTIISRMGQWYMIDICLGYKGKRKIHTVIYDSLKKLPFPVKKIAK
DFKLTVLKGDIDYHKERPVGYKITPEEYAYIKNDIQIIAEALLIQ
FKQGLDRMTAGSDSLKGFKDIITTKKFKKVFPTLSLGLDKEVRYA
YRGGFTWLNDRFKEKEIGEGMVFDVNSLYPAQMYSRLLPYGEPIV
FEGKYVWDEDYPLHIQHIRCEFELKEGYIPTIQIKRSRFYKGNEY
LKSSGGEIADLWLSNVDLELMKEHYDLYNVEYISGLKFKATTGLF
KDFIDKWTYIKTTSEGAIKQLAKLMLNSLYGKFASNPDVTGKVPY
LKENGALGFRLGEEETKDPVYTPMGVFITAWARYTTITAAQACYD
RIIYCDTDSIHLTGTEIPDVIKDIVDPKKLGYWAHESTFKRAKYL
RQKTYIQDIYMKEVDGKLVEGSPDDYTDIKFSVKCAGMTDKIKKE
VTFENFKVGFSRKMKPKPVQVPGGVVLVDDTFTIKSGGSAWSHPQ
FEKGGGSGGGSGGSAWSHPQFEK
Trwc Cba helicase
SEQ ID NO: 6
MLSVANVRSPSAAASYFASDNYYASADADRSGQWIGDGAKRLGLE
GKVEARAFDALLRGELPDGSSVGNPGQAHRPGTDLTFSVPKSWSL
LALVGKDERIIAAYREAVVEALHWAEKNAAETRVVEKGMVVTQAT
GNLAIGLFQHDTNRNQEPNLHFHAVIANVTQGKDGKWRTLKNDRL
WQLNTTLNSIAMARFRVAVEKLGYEPGPVLKHGNFEARGISREQV
MAFSTRRKEVLEARRGPGLDAGRIAALDTRASKEGIEDRATLSKQ
WSEAAQSIGLDLKPLVDRARTKALGQGMEATRIGSLVERGRAWLS
RFAAHVRGDPADPLVPPSVLKQDRQTIAAAQAVASAVRHLSQREA
AFERTALYKAALDFGLPTTIADVEKRTRALVRSGDLIAGKGEHKG
WLASRDAVVTEQRILSEVAAGKGDSSPAITPQKAAASVQAAALTG
QGFRLNEGQLAAARLILISKDRTIAVQGIAGAGKSSVLKPVAEVL
RDEGHPVIGLAIQNTLVQMLERDTGIGSQTLARFLGGWNKLLDDP
GNVALRAEAQASLKDHVLVLDEASMVSNEDKEKLVRLANLAGVHR
LVLIGDRKQLGAVDAGKPFALLQRAGIARAEMATNLRARDPVVRE
AQAAAQAGDVRKALRHLKSHTVEARGDGAQVAAETWLALDKETRA
RTSIYASGRAIRSAVNAAVQQGLLASREIGPAKMKLEVLDRVNTT
REELRHLPAYRAGRVLEVSRKQQALGLFIGEYRVIGQDRKGKLVE
VEDKRGKRFRFDPARIRAGKGDDNLTLLEPRKLEIHEGDRIRWTR
NDHRRGLENADQARVVEIANGKVTFETSKGDLVELKKDDPMLKRI
DLAYALNVHMAQGLTSDRGIAVMDSRERNLSNQKTFLVTVTRLRD
HLTLVVDSADKLGAAVARNKGEKASAIEVTGSVKPTATKGSGVDQ
PKSVEANKAEKELTRSKSKTLDFGI
Hel308 Mbu helicase
SEQ ID NO: 7
MMIRELDIPRDIIGFYEDSGIKELYPPQAEAIEMGLLEKKNLLAA
IPTASGKTLLAELAMIKAIREGGKALYIVPLRALASEKFERFKEL
APFGIKVGISTGDLDSRADWLGVNDIIVATSEKTDSLLRNGTSWM
DEITTVVVDEIHLLDSKNRGPTLEVTITKLMRLNPDVQVVALSAT
VGNAREMADWLGAALVLSEWRPTDLHEGVLFGDAINFPGSQKKID
RLEKDDAVNLVLDTIKAEGQCLVFESSRRNCAGFAKTASSKVAKI
LDNDIMIKLAGIAEEVESTGETDTAIVLANCIRKGVAFHHAGLNS
NHRKLVENGFRQNLIKVISSTPTLAAGLNLPARRVIIRSYRRFD
SNFGMQPIPVLEYKQMAGRAGRPHLDPYGESVLLAKTYDEFAQLM
ENYVEADAEDIWSKLGTENALRTHVLSTIVNGFASTRQELFDFFG
ATFFAYQQDKWMLEEVINDCLEFLIDKAMVSETEDIEDASKLFLR
GTRLGSLVSMLYIDPLSGSKIVDGFKDIGKSTGGNMGSLEDDKGD
DITVTDMTLLHLVCSTPDMRQLYLRNTDYTIVNEYIVAHSDEFHE
IPDKLKETDYEWFMGEVKTAMLLEEWVTEVSAEDITRHFNVGEGD
IHALADTSEWLMHAAAKLAELLGVEYSSHAYSLEKRIRYGSGLDL
MELVGIRGVGRVRARKLYNAGFVSVAKLKGADISVLSKLVGPKVA
YNILSGIGVRVNDKHENSAPISSNTLDTLLDKNQKTENDFQ
Dda helicase
SEQ ID NO: 8
MTFDDLTEGQKNAFNIVMKAIKEKKHHVTINGPAGTGKTTLTKFI
IEALISTGETGIILAAPTHAAKKILSKLSGKEASTIHSILKINPV
TYEENVLFEQKEVPDLAKCRVLICDEVSMYDRKLFKILLSTIPPW
CTIIGIGDNKQIRPVDPGENTAYISPFFTHKDFYQCELTEVKRSN
APIIDVATDVRNGKWIYDKVVDGHGVRGFTGDTALRDFMVNYFSI
VKSLDDLFENRVMAFTNKSVDKLNSIIRKKIFETDKDFIVGEIIV
MQEPLFKTYKIDGKPVSEIIFNNGQLVRIIEAEYTSTFVKARGVP
GEYLIRHWDLTVETYGDDEYYREKIKIISSDEELYKFNLFLGKTA
ETYKNWNKGGKAPWSDFWDAKSQFSKVKALPASTFHKAQGMSVDR
AFIYTPCIHYADVELAQQLLYVGVTRGRYDVFYV

1. A method for detecting multiple molecules in a sample, the method comprising:
(a) contacting the sample with a carrier and a nanopore, wherein the carrier comprises a single-stranded leader, an identifier region, and a molecule-binding region specific for a molecule to be detected, and wherein a motor protein is bound to the carrier such that it can control the movement of the identifier region within the nanopore;
(b) taking one or more optical or electrical measurements as a carrier moves within the nanopore to characterise the identifier region and to determine whether or not the molecule is bound to the molecule-binding region.

2. The method of claim 1, wherein the carrier further comprises a spacer between the bound motor protein and the molecule-binding region.

3. The method of claim 2, wherein the carrier comprises, in order, a single-stranded leader, an identifier region, a spacer and a molecule-binding region.

4. The method of claim 1, wherein the molecule-binding region and/or the identifier region is a polynucleotide.

5. The method of claim 1, wherein the molecule-binding region or a part thereof is the identifier region, optionally wherein the identifier region is a polynucleotide and comprises a barcode sequence.

6. (canceled)

7. The method of claim 1, wherein the carrier comprises more than one identifier region and/or more than one molecule-binding region.

8. (canceled)

9. The method of claim 1, wherein the molecules comprise neurotransmitters, proteins and/or miRNAs.

10. (canceled)

11. The method of claim 1, wherein the molecule-binding region is an aptamer, antibody, antibody fragment, nanobody or affibody.

12. The method of claim 1, wherein the molecule-binding region is complementary to a miRNA.

13. The method of claim 1, wherein the identifier region is a polynucleotide and the method comprises determining the polynucleotide sequence of the identifier region.

14. The method of claim 1, wherein the method is used to detect the presence or absence of the molecules.

15. The method of claim 1, wherein the method is used to determine the concentration of the molecules.

16. The method of claim 1, wherein the multiple molecules are 10 or more different molecules.

17. The method of claim 1, wherein the motor protein is a helicase, a polymerase, a nuclease, a translocase or a topoisomerase.

18. The method of claim 1, wherein the nanopore is a protein pore, a solid state pore, or a DNA origami pore.

19. A carrier comprising a single-stranded leader, an identifier region, and a molecule-binding region specific for a molecule to be detected, wherein a motor protein is bound to the carrier at a position between the single-stranded leader and the identifier region.

20. The carrier of claim 19, further comprising a spacer between the identifier region and the molecule-binding region and/or a molecule specifically bound to the molecule-binding region.

21. A population of carriers for multiple molecules, wherein the carriers are as defined in claim 19, and different carriers in the population comprise different identifier regions and different molecule-binding regions.

22. A kit for detecting multiple molecules in a sample comprising:
(i) a population of carriers, wherein each carrier comprises an identifier region and a molecule-binding region specific for a molecule to be detected, and different carriers in the population comprise different identifier regions and different molecule-binding regions;
(ii) an adaptor comprising a single-stranded leader; and
(iii) a motor protein.

23. A system for detecting multiple molecules in a sample comprising:
(i) a population of carriers according to claim 21; and
(iii) a nanopore.


SEQUENCE LISTING (229 sequences)

SEQ ID NO: 1 - protein, 485 aa, Escherichia coli
MMNDGKQQSTFLFHDYETFGTHPALDRPAQFAAIRTDSEFNVIGEPEVFYCKPADDYLPQPGAVLITGITPQEARAKGENEAAFAARIHSLFTVPKTCILGYNNVRFDDEVTRNIFYRNFYDPYAWSWQHDNSRWDLLDVMRACYALRPEGINWPENDDGLPSFRLEHLTKANGIEHSNAHDAMADVYATIAMAKLVKTRQPRLFDYLFTHRNKHKLMALIDVPQMKPLVHVSGMFGAWRGNTSWVAPLAWHPENRNAVIMVDLAGDISPLLELDSDTLRERLYTAKTDLGDNAAVPVKLVHINKCPVLAQANTLRPEDADRLGINRQHCLDNLKILRENPQVREKVVAIFAEAEPFTPSDNVDAQLYNGFFSDADRAAMKIVLETEPRNLPALDITFVDKRIEKLLFNYRARNFPGTLDYAEQQRWLEHRRQVFTPEFLQGYADELQMLVQQYADDKEKVALLKALWQYAEEIVSGSGHHHHHH

SEQ ID NO: 2 - protein, 268 aa, Escherichia coli
MKFVSFNINGLRARPHQLEAIVEKHQPDVIGLQETKVHDDMFPLEEVAKLGYNVFYHGQKGHYGVALLTKETPIAVRRGFPGDDEEAQRRIIMAEIPSLLGNVTVINGYFPQGESRDHPIKFPAKAQFYQNLQNYLETELKRDNPVLIMGDMNISPTDLDIGIGEENRKRWLRTGKCSFLPEEREWMDRLMSWGLVDTFRHANPQTADRFSWFDYRSKGFDDNRGLRIDLLLASQPLAECCVETGIDYEIRSMEKPSDHAPVWATFRR

SEQ ID NO: 3 - protein, 425 aa, Escherichia coli
MFRRKEDLDPPLALLPLKGLREAAALLEEALRQGKRIRVHGDYDADGLTGTAILVRGLAALGADVHPFIPHRLEEGYGVLMERVPEHLEASDLFLTVDCGITNHAELRELLENGVEVIVTDHHTPGKTPPPGLVVHPALTPDLKEKPTGAGVAFLLLWALHERLGLPPPLEYADLAAVGTIADVAPLWGWNRALVKEGLARIPASSWVGLRLLAEAVGYTGKAVEVAFRIAPRINAASRLGEAEKALRLLLTDDAAEAQALVGELHRLNARRQTLEEAMLRKLLPQADPEAKAIVLLDPEGHPGVMGIVASRILEATLRPVFLVAQGKGTVRSLAPISAVEALRSAEDLLLRYGGHKEAAGFAMDEALFPAFKARVEAYAARFPDPVREVALLDLLPEPGLLPQVFRELALLEPYGEGNPEPLFL

SEQ ID NO: 4 - protein, 226 aa, Bacteriophage lambda
MTPDIILQRTGIDVRAVEQGDDAWHKLRLGVITASEVHNVIAKPRSGKKWPDMKMSYFHTLLAEVCTGVAPEVNAKALAWGKQYENDARTLFEFTSGVNVTESPIIYRDESMRTACSPDGLCSDGNGLELKCPFTSRDFMKFRLGGFEAIKSAYMAQVQYSMWVTRKNAWYFANYDPRMKREGLHYVVIERDEKYMASFDEIVPEFIEKMDEALAEIGFVFGEQWR

SEQ ID NO: 5 - protein, 608 aa, Bacteriophage phi-29
MKHMPRKMYSCAFETTTKVEDCRVWAYGYMNIEDHSEYKIGNSLDEFMAWVLKVQADLYFHNLKFDGAFIINWLERNGFKWSADGLPNTYNTIISRMGQWYMIDICLGYKGKRKIHTVIYDSLKKLPFPVKKIAKDFKLTVLKGDIDYHKERPVGYKITPEEYAYIKNDIQIIAEALLIQFKQGLDRMTAGSDSLKGFKDIITTKKFKKVFPTLSLGLDKEVRYAYRGGFTWLNDRFKEKEIGEGMVFDVNSLYPAQMYSRLLPYGEPIVFEGKYVWDEDYPLHIQHIRCEFELKEGYIPTIQIKRSRFYKGNEYLKSSGGEIADLWLSNVDLELMKEHYDLYNVEYISGLKFKATTGLFKDFIDKWTYIKTTSEGAIKQLAKLMLNSLYGKFASNPDVTGKVPYLKENGALGFRLGEEETKDPVYTPMGVFITAWARYTTITAAQACYDRIIYCDTDSIHLTGTEIPDVIKDIVDPKKLGYWAHESTFKRAKYLRQKTYIQDIYMKEVDGKLVEGSPDDYTDIKFSVKCAGMTDKIKKEVTFENFKVGFSRKMKPKPVQVPGGVVLVDDTFTIKSGGSAWSHPQFEKGGGSGGGSGGSAWSHPQFEK

SEQ ID NO: 6 - protein, 970 aa, Artificial Sequence
  Trwc Cba helicase
MLSVANVRSPSAAASYFASDNYYASADADRSGQWIGDGAKRLGLEGKVEARAFDALLRGELPDGSSVGNPGQAHRPGTDLTFSVPKSWSLLALVGKDERIIAAYREAVVEALHWAEKNAAETRVVEKGMVVTQATGNLAIGLFQHDTNRNQEPNLHFHAVIANVTQGKDGKWRTLKNDRLWQLNTTLNSIAMARFRVAVEKLGYEPGPVLKHGNFEARGISREQVMAFSTRRKEVLEARRGPGLDAGRIAALDTRASKEGIEDRATLSKQWSEAAQSIGLDLKPLVDRARTKALGQGMEATRIGSLVERGRAWLSRFAAHVRGDPADPLVPPSVLKQDRQTIAAAQAVASAVRHLSQREAAFERTALYKAALDFGLPTTIADVEKRTRALVRSGDLIAGKGEHKGWLASRDAVVTEQRILSEVAAGKGDSSPAITPQKAAASVQAAALTGQGFRLNEGQLAAARLILISKDRTIAVQGIAGAGKSSVLKPVAEVLRDEGHPVIGLAIQNTLVQMLERDTGIGSQTLARFLGGWNKLLDDPGNVALRAEAQASLKDHVLVLDEASMVSNEDKEKLVRLANLAGVHRLVLIGDRKQLGAVDAGKPFALLQRAGIARAEMATNLRARDPVVREAQAAAQAGDVRKALRHLKSHTVEARGDGAQVAAETWLALDKETRARTSIYASGRAIRSAVNAAVQQGLLASREIGPAKMKLEVLDRVNTTREELRHLPAYRAGRVLEVSRKQQALGLFIGEYRVIGQDRKGKLVEVEDKRGKRFRFDPARIRAGKGDDNLTLLEPRKLEIHEGDRIRWTRNDHRRGLFNADQARVVEIANGKVTFETSKGDLVELKKDDPMLKRIDLAYALNVHMAQGLTSDRGIAVMDSRERNLSNQKTFLVTVTRLRDHLTLVVDSADKLGAAVARNKGEKASAIEVTGSVKPTATKGSGVDQPKSVEANKAEKELTRSKSKTLDFGI

SEQ ID NO: 7 - protein, 760 aa, Artificial Sequence
  Hel308 Mbu helicase
MMIRELDIPRDIIGFYEDSGIKELYPPQAEAIEMGLLEKKNLLAAIPTASGKTLLAELAMIKAIREGGKALYIVPLRALASEKFERFKELAPFGIKVGISTGDLDSRADWLGVNDIIVATSEKTDSLLRNGTSWMDEITTVVVDEIHLLDSKNRGPTLEVTITKLMRLNPDVQVVALSATVGNAREMADWLGAALVLSEWRPTDLHEGVLFGDAINFPGSQKKIDRLEKDDAVNLVLDTIKAEGQCLVFESSRRNCAGFAKTASSKVAKILDNDIMIKLAGIAEEVESTGETDTAIVLANCIRKGVAFHHAGLNSNHRKLVENGFRQNLIKVISSTPTLAAGLNLPARRVIIRSYRRFDSNFGMQPIPVLEYKQMAGRAGRPHLDPYGESVLLAKTYDEFAQLMENYVEADAEDIWSKLGTENALRTHVLSTIVNGFASTRQELFDFFGATFFAYQQDKWMLEEVINDCLEFLIDKAMVSETEDIEDASKLFLRGTRLGSLVSMLYIDPLSGSKIVDGFKDIGKSTGGNMGSLEDDKGDDITVTDMTLLHLVCSTPDMRQLYLRNTDYTIVNEYIVAHSDEFHEIPDKLKETDYEWFMGEVKTAMLLEEWVTEVSAEDITRHFNVGEGDIHALADTSEWLMHAAAKLAELLGVEYSSHAYSLEKRIRYGSGLDLMELVGIRGVGRVRARKLYNAGFVSVAKLKGADISVLSKLVGPKVAYNILSGIGVRVNDKHFNSAPISSNTLDTLLDKNQKTFNDFQ

SEQ ID NO: 8 - protein, 439 aa, Artificial Sequence
  Dda helicase
MTFDDLTEGQKNAFNIVMKAIKEKKHHVTINGPAGTGKTTLTKFIIEALISTGETGIILAAPTHAAKKILSKLSGKEASTIHSILKINPVTYEENVLFEQKEVPDLAKCRVLICDEVSMYDRKLFKILLSTIPPWCTIIGIGDNKQIRPVDPGENTAYISPFFTHKDFYQCELTEVKRSNAPIIDVATDVRNGKWIYDKVVDGHGVRGFTGDTALRDFMVNYFSIVKSLDDLFENRVMAFTNKSVDKLNSIIRKKIFETDKDFIVGEIIVMQEPLFKTYKIDGKPVSEIIFNNGQLVRIIEAEYTSTFVKARGVPGEYLIRHWDLTVETYGDDEYYREKIKIISSDEELYKFNLFLGKTAETYKNWNKGGKAPWSDFWDAKSQFSKVKALPASTFHKAQGMSVDRAFIYTPCIHYADVELAQQLLYVGVTRGRYDVFYV

SEQ ID NO: 9 - DNA, 35 bp, Artificial Sequence
  Barcode 1
tgctactctcctcataagcagtccggtgtatcgat

SEQ ID NO: 10 - DNA, 35 bp, Artificial Sequence
  Barcode 2
atcgctacgccttcggctcgtaatcatagtcgagt

SEQ ID NO: 11 - DNA, 35 bp, Artificial Sequence
  Barcode 3
agctcagagcaggtcactcaagatacgagctgcgt

SEQ ID NO: 12 - DNA, 35 bp, Artificial Sequence
  Barcode 4
gtaagtctgcatcagcgcgcggctgtgcgaggata

SEQ ID NO: 13 - DNA, 35 bp, Artificial Sequence
  Barcode 5
ctacgacagtacgctagcaaggatagacactacga

SEQ ID NO: 14 - DNA, 35 bp, Artificial Sequence
  Barcode 6
tactgaacacaagttcgtcgtcgagcaatcacaat

SEQ ID NO: 15 - DNA, 35 bp, Artificial Sequence
  Barcode 7
agtctaccattacttggatcggattagcctcactc

SEQ ID NO: 16 - DNA, 35 bp, Artificial Sequence
  Barcode 8
tgcacgagtgcgtgtcaaccgtccagatgctcgtg

SEQ ID NO: 17 - DNA, 35 bp, Artificial Sequence
  Barcode 9
ctagtgcgcagttgtctcggcggagttgagactga

SEQ ID NO: 18 - DNA, 35 bp, Artificial Sequence
  Barcode 10
gatcatggtagtcttcaagatcgagtatgtctgtc

SEQ ID NO: 19 - RNA, 21 nt, Artificial Sequence
  miRNA 1
cagcagcacacugugguuugu

SEQ ID NO: 20 - RNA, 22 nt, Artificial Sequence
  miRNA 2
agagcuuagcugauuggugaac

SEQ ID NO: 21 - RNA, 21 nt, Artificial Sequence
  miRNA 3
uagcuuaucagacugauguug

SEQ ID NO: 22 - RNA, 22 nt, Artificial Sequence
  miRNA 4
accuggcauacaauguagauuu

SEQ ID NO: 23 - RNA, 22 nt, Artificial Sequence
  miRNA 5
uguaaacauccccgacuggaag

SEQ ID NO: 24 - RNA, 23 nt, Artificial Sequence
  miRNA 6
uguaaacauccuacacucucagc

SEQ ID NO: 25 - RNA, 22 nt, Artificial Sequence
  miRNA 7
agcugguaaaauggaaccaaau

SEQ ID NO: 26 - RNA, 22 nt, Artificial Sequence
  miRNA 8
gagcuuuuggcccggguuauac

SEQ ID NO: 27 - RNA, 23 nt, Artificial Sequence
  miRNA 9
aacauucauugcugucggugggu

SEQ ID NO: 28 - RNA, 22 nt, Artificial Sequence
  miRNA 10
uagcaccaucugaaaucgguua

SEQ ID NO: 29 - DNA, 50 bp, Artificial Sequence
  Synthetic construct
  misc_feature (50)..(50): thymine attached to 2 x iSpC3 which is joined to another nucleic acid
cctagttccgctggggagctagcatacgtgtctaacactgcacagatgat

SEQ ID NO: 30 - DNA, 50 bp, Artificial Sequence
  Synthetic construct
  misc_feature (50)..(50): cytosine attached to 2 x iSpC3 which is joined to another nucleic acid
cctagttccgctgggatataggcataaagtaaaatcgtaccaactcaatc

SEQ ID NO: 31 - DNA, 50 bp, Artificial Sequence
  Synthetic construct
  misc_feature (50)..(50): thymine attached to 2 x iSpC3 which is joined to another nucleic acid
cctagttccgctgggctcggcgttgtgtgtcaaatggcgtagatctggat

SEQ ID NO: 32 - DNA, 35 bp, Artificial Sequence
  Synthetic construct
  misc_feature (35)..(35): thymine attached to 2 x iSp18 which is joined to another nucleic acid
cctagttccgctgggcacagccccatgtaacccat

SEQ ID NO: 33 - DNA, 50 bp, Artificial Sequence
  Synthetic construct
  misc_feature (50)..(50): thymine attached to 2 x iSpC3 which is joined to another nucleic acid
cctagttccgctgggactagataaaaggaagggagcacagtaacgtcgtt

SEQ ID NO: 34 - DNA, 50 bp, Artificial Sequence
  Synthetic construct
  misc_feature (50)..(50): adenine attached to 2 x iSpC3 which is joined to another nucleic acid
cctagttccgctgggtctcccgtatccgtggctaaacgccttcaatctta

SEQ ID NO: 35 - DNA, 40 bp, Artificial Sequence
  Synthetic construct
  misc_feature (40)..(40): adenine attached to 2 x iSpC3 which is joined to another nucleic acid
cctagttccgctgggaccttttctaggatggaacattcta

SEQ ID NO: 36 - DNA, 51 bp, Artificial Sequence
  Synthetic construct
  misc_feature (51)..(51): thymine attached to 2 x iSpC3 which is joined to another nucleic acid
cctagttccgctgggtgctactcttcctcataagcagtccggtgtatcgat

SEQ ID NO: 37 - DNA, 50 bp, Artificial Sequence
  Synthetic construct
  misc_feature (50)..(50): thymine attached to 2 x iSpC3 which is joined to another nucleic acid
cctagttccgctgggatcgctacgccttcggctcgtaatcatagtcgagt

SEQ ID NO: 38 - DNA, 50 bp, Artificial Sequence
  Synthetic construct
  misc_feature (50)..(50): thymine attached to 2 x iSpC3 which is joined to another nucleic acid
cctagttccgctgggagctcagagcaggtcactcaagatacgagctgcgt

SEQ ID NO: 39 - DNA, 50 bp, Artificial Sequence
  Synthetic construct
  misc_feature (50)..(50): adenine attached to 2 x iSpC3 which is joined to another nucleic acid
cctagttccgctggggtaagtctgcatcagcgcgcggctgtgcgaggata

SEQ ID NO: 40 - DNA, 50 bp, Artificial Sequence
  Synthetic construct
  misc_feature (50)..(50): adenine attached to 2 x iSpC3 which is joined to another nucleic acid
cctagttccgctgggctacgacagtacgctagcaaggatagacactacga

SEQ ID NO: 41 - DNA, 50 bp, Artificial Sequence
  Synthetic construct
  misc_feature (50)..(50): thymine attached to 2 x iSpC3 which is joined to another nucleic acid
cctagttccgctgggtactgaacacaagttcgtcgtcgagcaatcacaat

SEQ ID NO: 42 - DNA, 50 bp, Artificial Sequence
  Synthetic construct
  misc_feature (50)..(50): cytosine attached to 2 x iSpC3 which is joined to another nucleic acid
cctagttccgctgggagtctaccattacttggatcggattagcctcactc

SEQ ID NO: 43 - DNA, 50 bp, Artificial Sequence
  Synthetic construct
  misc_feature (50)..(50): guanine attached to 2 x iSpC3 which is joined to another nucleic acid
cctagttccgctgggtgcacgagtgcgtgtcaaccgtccagatgctcgtg

SEQ ID NO: 44 - DNA, 50 bp, Artificial Sequence
  Synthetic construct
  misc_feature (50)..(50): adenine attached to 2 x iSpC3 which is joined to another nucleic acid
cctagttccgctgggctagtgcgcagttgtctcggcggagttgagactga

SEQ ID NO: 45 - DNA, 50 bp, Artificial Sequence
  Synthetic construct
  misc_feature (50)..(50): cytosine attached to 2 x iSpC3 which is joined to another nucleic acid
cctagttccgctggggatcatggtagtcttcaagatcgagtatgtctgtc

SEQ ID NO: 46 - DNA, 73 bp, Artificial Sequence
  Synthetic construct
cctagttccgctgggtactgaacacaagttcgtcgtcgagcaatcacaatgctgagagtgtaggatgtttaca

SEQ ID NO: 47 - DNA, 72 bp, Artificial Sequence
  Synthetic construct
cctagttccgctgggcgtgaagagagtttcataatacgtccagccgcatgtaaccgatttcagatggtgcta

SEQ ID NO: 48 - DNA, 13 bp, Artificial Sequence
  Synthetic construct
  misc_feature (13)..(13): adenine attached to spacer which is joined to another nucleic acid
tcgatacaataca

SEQ ID NO: 49 - DNA, 76 bp, Artificial Sequence
  Synthetic construct
agtctccgctgtcctcccgatgcacttgacgtatgtctcactttcttttcattgacatgggatgacgccgtgactg

SEQ ID NO: 50 - DNA, 50 bp, Artificial Sequence; identical to SEQ ID NO: 37
  Synthetic construct
  misc_feature (50)..(50): thymine attached to 2 x iSpC3 which is joined to another nucleic acid

SEQ ID NO: 51 - DNA, 50 bp, Artificial Sequence; identical to SEQ ID NO: 38
  Synthetic construct
  misc_feature (50)..(50): thymine attached to 2 x iSpC3 which is joined to another nucleic acid

SEQ ID NO: 52 - DNA, 50 bp, Artificial Sequence; identical to SEQ ID NO: 39
  Synthetic construct
  misc_feature (50)..(50): adenine attached to 2 x iSpC3 which is joined to another nucleic acid

SEQ ID NO: 53 - DNA, 50 bp, Artificial Sequence; identical to SEQ ID NO: 40
  Synthetic construct
  misc_feature (50)..(50): adenine attached to 2 x iSpC3 which is joined to another nucleic acid

SEQ ID NO: 54 - DNA, 50 bp, Artificial Sequence; identical to SEQ ID NO: 41
  Synthetic construct
  misc_feature (50)..(50): thymine attached to 2 x iSpC3 which is joined to another nucleic acid

SEQ ID NO: 55 - DNA, 50 bp, Artificial Sequence; identical to SEQ ID NO: 44
  Synthetic construct
  misc_feature (50)..(50): adenine attached to 2 x iSpC3 which is joined to another nucleic acid

SEQ ID NO: 56 - DNA, 50 bp, Artificial Sequence; identical to SEQ ID NO: 45
  Synthetic construct
  misc_feature (50)..(50): cytosine attached to 2 x iSpC3 which is joined to another nucleic acid

SEQ ID NO: 57 - DNA, 50 bp, Artificial Sequence
  Synthetic construct
  misc_feature (50)..(50): adenine attached to 2 x iSpC3 which is joined to another nucleic acid
cctagttccgctggggttcacatcaaggtcataccgcgagttctatttta

SEQ ID NO: 58 - DNA, 50 bp, Artificial Sequence
  Synthetic construct
  misc_feature (50)..(50): thymine attached to 2 x iSpC3 which is joined to another nucleic acid
cctagttccgctggggcttgggggatagatgtgccccgcgcatcggacct

SEQ ID NO: 59 - DNA, 50 bp, Artificial Sequence
  Synthetic construct
  misc_feature (50)..(50): adenine attached to 2 x iSpC3 which is joined to another nucleic acid
cctagttccgctgggatgacacacgttttcgatagggacgccgactttaa

SEQ ID NO: 60 - DNA, 50 bp, Artificial Sequence
  Synthetic construct
  misc_feature (50)..(50): cytosine attached to 2 x iSpC3 which is joined to another nucleic acid
cctagttccgctgggtgataataagacctgacagacaatagggagaactc

SEQ ID NO: 61 - DNA, 50 bp, Artificial Sequence
  Synthetic construct
  misc_feature (50)..(50): thymine attached to 2 x iSpC3 which is joined to another nucleic acid
cctagttccgctgggttagtaatcaagtctgatcgtaatagctaagtcat

SEQ ID NO: 62 - DNA, 50 bp, Artificial Sequence
  Synthetic construct
  misc_feature (50)..(50): thymine attached to 2 x iSp3C which is joined to another nucleic acid
cctagttccgctgggcctgttctaattgcgcggagaggcgagatgtttct

SEQ ID NO: 63 - DNA, 50 bp, Artificial Sequence
  Synthetic construct
  misc_feature (50)..(50): cytosine attached to 2 x iSpC3 which is joined to another nucleic acid
cctagttccgctgggtgggatactgacgtgccggaatacccagacgtgcc

SEQ ID NO: 64 - DNA, 50 bp, Artificial Sequence
  Synthetic construct
  misc_feature (50)..(50): guanine attached to 2 x iSpC3 which is joined to another nucleic acid
cctagttccgctgggtgatcggcacctaaacgcattagccctgcaatacg

SEQ ID NO: 65 - DNA, 50 bp, Artificial Sequence
  Synthetic construct
  misc_feature (50)..(50): guanine attached to 2 x iSpC3 which is joined to another nucleic acid
cctagttccgctgggagctgctcggaagccataaggtactttaatttggg

SEQ ID NO: 66 - DNA, 50 bp, Artificial Sequence
  Synthetic construct
  misc_feature (50)..(50): adenine attached to 2 x iSpC3 which is joined to another nucleic acid
cctagttccgctgggcacggatttctatattgctcaaccaggcagcgcaa

SEQ ID NO: 67 - DNA, 50 bp, Artificial Sequence
  Synthetic construct
  misc_feature (50)..(50): cytosine attached to 2 x iSpC3 which is joined to another nucleic acid
cctagttccgctgggattgtgcgcttttctccatgcgtcttagacatctc

SEQ ID NO: 68 - DNA, 50 bp, Artificial Sequence
  Synthetic construct
  misc_feature (50)..(50): thymine attached to 2 x iSpC3 which is joined to another nucleic acid
cctagttccgctggggtgaaatccccgtctaggttatggctggggggatt

SEQ ID NO: 69 - DNA, 50 bp, Artificial Sequence
  Synthetic construct
  misc_feature (50)..(50): adenine attached to 2 x iSpC3 which is joined to another nucleic acid
cctagttccgctgggcgtaaacttatcacgacacaatgaacaagcctgca

SEQ ID NO: 70 - DNA, 50 bp, Artificial Sequence
  Synthetic construct
  misc_feature (50)..(50): cytosine attached to 2 x iSpC3 which is joined to another nucleic acid
cctagttccgctgggggcagtgtccgagcgtcctcaatcatgagcgattc

SEQ ID NO: 71 - DNA, 50 bp, Artificial Sequence
  Synthetic construct
  misc_feature (50)..(50): adenine attached to 2 x iSpC3 which is joined to another nucleic acid
cctagttccgctgggcgtccagacttaatgtctgctcactgacatcgcga

SEQ ID NO: 72 - DNA, 50 bp, Artificial Sequence
  Synthetic construct
  misc_feature (50)..(50): guanine attached to 2 x iSpC3 which is joined to another nucleic acid
cctagttccgctgggattagcggaaccaaacccaggaaggcttgaaggcg

SEQ ID NO: 73 - DNA, 50 bp, Artificial Sequence
  Synthetic construct
  misc_feature (50)..(50): guanine attached to 2 x iSpC3 which is joined to another nucleic acid
cctagttccgctgggaaccttaggggcctcgaatctttgagacgactagg

SEQ ID NO: 74 - DNA, 50 bp, Artificial Sequence
  Synthetic construct
  misc_feature (50)..(50): thymine attached to 2 x iSpC3 which is joined to another nucleic acid
cctagttccgctgggtaattactgccccaccatgacattttaatagcagt

SEQ ID NO: 75 - DNA, 50 bp, Artificial Sequence
  Synthetic construct
  misc_feature (50)..(50): adenine attached to 2 x iSpC3 which is joined to another nucleic acid
cctagttccgctggggaccttgagacagaacttatcaatgtacaactgaa

SEQ ID NO: 76 - DNA, 50 bp, Artificial Sequence
  Synthetic construct
  misc_feature (50)..(50): guanine attached to 2 x iSpC3 which is joined to another nucleic acid
cctagttccgctgggcgaaggattggccccccgattaccaccgccgtgag

SEQ ID NO: 77 - DNA, 50 bp, Artificial Sequence
  Synthetic construct
  misc_feature (50)..(50): cytosine attached to 2 x iSpC3 which is joined to another nucleic acid
cctagttccgctgggaattgccaacaggtcaagccctgttctcactggtc

SEQ ID NO: 78 - DNA, 50 bp, Artificial Sequence
  Synthetic construct
  misc_feature (50)..(50): adenine attached to 2 x iSpC3 which is joined to another nucleic acid
cctagttccgctgggcagtgcttgcccccagtagagtgtggaagggcata

SEQ ID NO: 79 - DNA, 50 bp, Artificial Sequence
  Synthetic construct
  misc_feature (50)..(50): guanine attached to 2 x iSpC3 which is joined to another nucleic acid
cctagttccgctgggaaagcccactctccacacttcaaggttaaatggcg

SEQ ID NO: 80 - DNA, 50 bp, Artificial Sequence
  Synthetic construct
  misc_feature (50)..(50): cytosine attached to 2 x iSpC3 which is joined to another nucleic acid
cctagttccgctggggactaatacaatcggaagcaactctcacgccgcac

SEQ ID NO: 81 - DNA, 50 bp, Artificial Sequence
  Synthetic construct
  misc_feature (50)..(50): cytosine attached to 2 x iSpC3 which is joined to another nucleic acid
cctagttccgctgggctacgattcatgtctccccccacatatgattgatc

SEQ ID NO: 82 - DNA, 50 bp, Artificial Sequence
  Synthetic construct
  misc_feature (50)..(50): adenine attached to 2 x iSpC3 which is joined to another nucleic acid
cctagttccgctgggtcgggcgcttaatcgcaatgttcatccggaacgga

SEQ ID NO: 83 - DNA, 50 bp, Artificial Sequence
  Synthetic construct
  misc_feature (50)..(50): guanine attached to 2 x iSpC3 which is joined to another nucleic acid
cctagttccgctgggtaaattccacattacgatagctgacgtcctggtag

SEQ ID NO: 84 - DNA, 50 bp, Artificial Sequence
  Synthetic construct
  misc_feature (50)..(50): thymine attached to 2 x iSpC3 which is joined to another nucleic acid
cctagttccgctgggcaccccctagcctgaccttaaatgataattcctgt

SEQ ID NO: 85 - DNA, 50 bp, Artificial Sequence
  Synthetic construct
  misc_feature (50)..(50): cytosine attached to 2 x iSpC3 which is joined to another nucleic acid
cctagttccgctgggtttagcctaacctctgatgcattgcaccggagttc

SEQ ID NO: 86 - DNA, 50 bp, Artificial Sequence
  Synthetic construct
  misc_feature (50)..(50): guanine attached to 2 x iSpC3 which is joined to another nucleic acid
cctagttccgctgggcccaatatacgctgaaccttccatccgattttcag

SEQ ID NO: 87 - DNA, 50 bp, Artificial Sequence
  Synthetic construct
  misc_feature (50)..(50): guanine attached to 2 x iSpC3 which is joined to another nucleic acid
cctagttccgctgggccttggaattagaaccgtgtgattctacgcctagg

SEQ ID NO: 88 - DNA, 50 bp, Artificial Sequence
  Synthetic construct
  misc_feature (50)..(50): thymine attached to 2 x iSpC3 which is joined to another nucleic acid
cctagttccgctgggaggacttcttcggtgtaatcggagtacatcaatgt

SEQ ID NO: 89 - DNA, 50 bp, Artificial Sequence
  Synthetic construct
  misc_feature (50)..(50): thymine attached to 2 x iSpC3 which is joined to another nucleic acid
cctagttccgctgggaacgtactttgtggggataagctgtacagggctgt

SEQ ID NO: 90 - DNA, 50 bp, Artificial Sequence
  Synthetic construct
  misc_feature (50)..(50): guanine attached to 2 x iSpC3 which is joined to another nucleic acid
cctagttccgctgggattcagatctttgaccgatcgtactgacgtgtacg

SEQ ID NO: 91 - DNA, 50 bp, Artificial Sequence
  Synthetic construct
  misc_feature (50)..(50): adenine attached to 2 x iSpC3 which is joined to another nucleic acid
cctagttccgctgggcttaagttcggtcattaacagtgtcaatcttgcca

SEQ ID NO: 92 - DNA, 50 bp, Artificial Sequence
  Synthetic construct
  misc_feature (50)..(50): adenine attached to 2 x iSpC3 which is joined to another nucleic acid
cctagttccgctgggatgtaattcctgctaggtccaatgtgactgtacta

SEQ ID NO: 93 - DNA, 50 bp, Artificial Sequence
  Synthetic construct
  misc_feature (50)..(50): adenine attached to 2 x iSpC3 which is joined to another nucleic acid
cctagttccgctgggttaagctattgctaactgtagtcctagtctagcta

SEQ ID NO: 94 - DNA, 50 bp, Artificial Sequence; identical to SEQ ID NO: 29
  Synthetic construct
  misc_feature (50)..(50): thymine attached to 2 x iSpC3 which is joined to another nucleic acid

SEQ ID NO: 95 - DNA, 40 bp, Artificial Sequence; identical to SEQ ID NO: 35
  Synthetic construct
  misc_feature (40)..(40): adenine attached to 2 x iSpC3 which is joined to another nucleic acid

SEQ ID NO: 96 - DNA, 40 bp, Artificial Sequence
  Synthetic construct
  misc_feature (40)..(40): cytosine attached to 2 x iSpC3 which is joined to another nucleic acid
cctagttccgctgggctgcgacatgactatctagagtcgc

SEQ ID NO: 97 - DNA, 50 bp, Artificial Sequence; identical to SEQ ID NO: 33
  Synthetic construct
  misc_feature (50)..(50): thymine attached to 2 x iSpC3 which is joined to another nucleic acid

SEQ ID NO: 98 - DNA, 28 bp, Artificial Sequence
  Synthetic construct
  misc_feature (28)..(28): adenine attached to 2 x iSpC3 which is joined to another nucleic acid
cctagttccgctgggtcgatacaataca

SEQ ID NO: 99 - RNA, 21 nt, Artificial Sequence
  miRNA 11
cagcagcacacugugguuugu

SEQ ID NO: 100 - RNA, 22 nt, Artificial Sequence
  miRNA 12
agagcuuagcugauuggugaac

SEQ ID NO: 101 - RNA, 22 nt, Artificial Sequence
  miRNA 13
uagcuuaucagacugauguuga

SEQ ID NO: 102 - RNA, 22 nt, Artificial Sequence
  miRNA 14
accuggcauacaauguagauuu

SEQ ID NO: 103 - RNA, 22 nt, Artificial Sequence
  miRNA 15
uguaaacauccccgacuggaag

SEQ ID NO: 104 - RNA, 23 nt, Artificial Sequence
  miRNA 16
uguaaacauccuacacucucagc

SEQ ID NO: 105 - RNA, 22 nt, Artificial Sequence
  miRNA 17
agcugguaaaauggaaccaaau

SEQ ID NO: 106 - RNA, 22 nt, Artificial Sequence
  miRNA 18
gagcuuuuggcccggguuauac

SEQ ID NO: 107 - RNA, 23 nt, Artificial Sequence
  miRNA 19
aacauucauugcugucggugggu

SEQ ID NO: 108 - RNA, 22 nt, Artificial Sequence
  miRNA 20
uagcaccaucugaaaucgguua

SEQ ID NO: 109 - RNA, 22 nt, Artificial Sequence
  miRNA 22
agccccugcccaccgcacacug

SEQ ID NO: 110 - RNA, 21 nt, Artificial Sequence
  miRNA 24
cauuauuacuuuugguacgcg

SEQ ID NO: 111 - RNA, 22 nt, Artificial Sequence
  miRNA 25
ccaccuccccugcaaacgucca

SEQ ID NO: 112 - RNA, 21 nt, Artificial Sequence
  miRNA 26
cauuauuacuuuugguacgcg

SEQ ID NO: 113 - RNA, 24 nt, Artificial Sequence
  miRNA 27
agccuggaagcuggagccugcagu

SEQ ID NO: 114 - RNA, 22 nt, Artificial Sequence
  miRNA 29
uguaaacauccuugacuggaag

SEQ ID NO: 115 - RNA, 23 nt, Artificial Sequence
  miRNA 30
aaaagugcuuacagugcagguag

SEQ ID NO: 116 - RNA, 22 nt, Artificial Sequence
  miRNA 31
acaguagucugcacauugguua

SEQ ID NO: 117 - RNA, 21 nt, Artificial Sequence
  miRNA 32
aauggcgccacuaggguugug

SEQ ID NO: 118 - RNA, 21 nt, Artificial Sequence
  miRNA 33
uucaaguaauucaggauaggu

SEQ ID NO: 119 - RNA, 23 nt, Artificial Sequence
  miRNA 34
guccaguuuucccaggaaucccu

SEQ ID NO: 120 - RNA, 22 nt, Artificial Sequence
  miRNA 35
uauugcacuugucccggccugu

SEQ ID NO: 121 - RNA, 22 nt, Artificial Sequence
  miRNA 36
ugagaacugaauuccauggguu

SEQ ID NO: 122 - RNA, 23 nt, Artificial Sequence
  miRNA 37
ugaggggcagagagcgagacuuu

SEQ ID NO: 123 - RNA, 21 nt, Artificial Sequence
  miRNA 39
uucacaguggcuaaguucugc

SEQ ID NO: 124 - RNA, 22 nt, Artificial Sequence
  miRNA 40
uggaauguaaagaaguauguau

SEQ ID NO: 125 - RNA, 23 nt, Artificial Sequence
  miRNA 42
uaaggugcaucuagugcagauag

SEQ ID NO: 126 - RNA, 23 nt, Artificial Sequence
  miRNA 43
uaaggugcaucuagugcaguuag

SEQ ID NO: 127 - RNA, 22 nt, Artificial Sequence
  miRNA 45
gcucugacuuuauugcacuacu

SEQ ID NO: 128 - RNA, 22 nt, Artificial Sequence
  miRNA 46
ugagguaguagguuguaugguu

SEQ ID NO: 129 - RNA, 24 nt, Artificial Sequence
  miRNA 47
ucccugagacccuuuaaccuguga

SEQ ID NO: 130 - RNA, 21 nt, Artificial Sequence
  miRNA 49
cuauauaucaaacauauuccu

SEQ ID NO: 131 - RNA, 22 nt, Artificial Sequence
  miRNA 50
aacuggcccucaaagucccgcu

SEQ ID NO: 132 - RNA, 22 nt, Artificial Sequence
  miRNA 51
ugggucuuugcgggcgagauga

SEQ ID NO: 133 - RNA, 22 nt, Artificial Sequence
  miRNA 52
uucccuuugucauccuucgccu

SEQ ID NO: 134 - RNA, 22 nt, Artificial Sequence
  miRNA 53
ucaguaaauguuuauuagauga

SEQ ID NO: 135 - RNA, 23 nt, Artificial Sequence
  miRNA 54
agugccugagggaguaagagccc

SEQ ID NO: 136 - RNA, 25 nt, Artificial Sequence
  miRNA 55
agggaucgcgggcggguggcggccu

SEQ ID NO: 137 - RNA, 23 nt, Artificial Sequence
  miRNA 56
aggaagcccuggaggggcuggag

SEQ ID NO: 138 - RNA, 22 nt, Artificial Sequence
  miRNA 57
agugggaggccagggcacggca

SEQ ID NO: 139 - RNA, 22 nt, Artificial Sequence
  miRNA 58
ggcuggagcgagugcaguggug

SEQ ID NO: 140 - RNA, 22 nt, Artificial Sequence
  miRNA 59
gagcaauguagguagacuguuu

SEQ ID NO: 141 - RNA, 21 nt, Artificial Sequence
  miRNA 60
caauucucaaaggagccuccc

SEQ ID NO: 142 - DNA, 59 bp, Artificial Sequence
  Synthetic construct
ttttttttttttttttttttttttttttttagtccgtggtagggcaggttggggtgact

SEQ ID NO: 143 - DNA, 74 bp, Artificial Sequence
  Synthetic construct
ttttttttttttttttttttttttttttttcgactggtaggcagataggggaagctgattcgatgcgtgggtcg

SEQ ID NO: 144 - DNA, 38 bp, Artificial Sequence
  Synthetic construct
gggtgctactctcctcataagcagtccggtgtatcgat

SEQ ID NO: 145 - DNA, 38 bp, Artificial Sequence
  Synthetic construct
gggatcgctacgccttcggctcgtaatcatagtcgagt

SEQ ID NO: 146 - DNA, 38 bp, Artificial Sequence
  Synthetic construct
gggagctcagagcaggtcactcaagatacgagctgcgt

SEQ ID NO: 147 - DNA, 38 bp, Artificial Sequence
  Synthetic construct
ggggtaagtctgcatcagcgcgcggctgtgcgaggata

SEQ ID NO: 148 - DNA, 38 bp, Artificial Sequence
  Synthetic construct
gggctacgacagtacgctagcaaggatagacactacga

SEQ ID NO: 149 - DNA, 38 bp, Artificial Sequence
  Synthetic construct
gggtactgaacacaagttcgtcgtcgagcaatcacaat

SEQ ID NO: 150 - DNA, 38 bp, Artificial Sequence
  Synthetic construct
gggagtctaccattacttggatcggattagcctcactc

SEQ ID NO: 151 - DNA, 38 bp, Artificial Sequence
  Synthetic construct
gggtgcacgagtgcgtgtcaaccgtccagatgctcgtg

SEQ ID NO: 152 - DNA, 38 bp, Artificial Sequence
  Synthetic construct
gggctagtgcgcagttgtctcggcggagttgagactga

SEQ ID NO: 153 - DNA, 38 bp, Artificial Sequence
  Synthetic construct
ggggatcatggtagtcttcaagatcgagtatgtctgtc

SEQ ID NO: 154 - DNA, 85 bp, Artificial Sequence
  Synthetic construct
atattgtacttccgtttcgaatttacgtattgctcctagttccgctggtactgaacacaagttcgtcgtcagtacaatacagtct

SEQ ID NO: 155 - DNA, 35 bp, Artificial Sequence
  Synthetic construct
  misc_feature (35)..(35): thymine attached to 2 x iSpC3 which is joined to another nucleic acid
actagataaaaggaagggagcacagtaacgtcgtt

SEQ ID NO: 156 - DNA, 13 bp, Artificial Sequence
  Synthetic construct
  misc_feature (13)..(13): adenine attached to 2 x iSpC3 which is joined to another nucleic acid
tcgatacaataca

SEQ ID NO: 157 - DNA, 16 bp, Artificial Sequence
  Synthetic construct
cccagcggaactagga

SEQ ID NO: 158 - DNA, 15 bp, Artificial Sequence
  Synthetic construct
  misc_feature (1)..(1): guanine attached to 2 x iSpC3 which is joined to another nucleic acid
ggttggtgtggttgg

SEQ ID NO: 159 - DNA, 15 bp, Artificial Sequence
  Synthetic construct
  misc_feature (1)..(1): guanine attached to 2 x iSpC3 which is joined to another nucleic acid
ggttggtgtggttgg

SEQ ID NO: 160 - DNA, 15 bp, Artificial Sequence
  Synthetic construct
  misc_feature (1)..(1): guanine attached to 2 x iSpC3 which is joined to another nucleic acid
ggttggtgtggttgg

SEQ ID NO: 161 - DNA, 29 bp, Artificial Sequence
  Synthetic construct
  misc_feature (1)..(1): adenine attached to 2 x iSp18 which is joined to another nucleic acid
agtccgtggtagggcaggttggggtgact

SEQ ID NO: 162 - DNA, 35 bp, Artificial Sequence
  Synthetic construct
  misc_feature (1)..(1): cytosine attached to 2 x iSpC3 which is joined to another nucleic acid
cgactggtaggcagataggggaagctgattcgatg

SEQ ID NO: 163 - DNA, 44 bp, Artificial Sequence
  Synthetic construct
  misc_feature (1)..(1): guanine attached to 2 x iSpC3 which is joined to another nucleic acid
ggatattgcgcgattccggtcggcagcttaggaagtgcggtgtc

SEQ ID NO: 164 - DNA, 56 bp, Artificial Sequence
  Synthetic construct
  misc_feature (1)..(1): adenine attached to 2 x iSpC3 which is joined to another nucleic acid
cacgcataacgtcttgcggggcggcgggttgagaggatgtcgggtggttatgcgtg

SEQ ID NO: 165 - DNA, 21 bp, Artificial Sequence
  Synthetic construct
  misc_feature (1)..(1): adenine attached to 2 x iSpC3 which is joined to another nucleic acid
acaaaccacagtgtgctgctg

SEQ ID NO: 166 - DNA, 22 bp, Artificial Sequence
  Synthetic construct
  misc_feature (1)..(1): guanine attached to 2 x iSpC3 which is joined to another nucleic acid
gttcaccaatcagctaagctct

SEQ ID NO: 167 - DNA, 22 bp, Artificial Sequence
  Synthetic construct
  misc_feature (1)..(1): thymine attached to 2 x iSpC3 which is joined to another nucleic acid
tcaacatcagtctgataagcta

SEQ ID NO: 168 - DNA, 22 bp, Artificial Sequence
  Synthetic construct
  misc_feature (1)..(1): adenine attached to 2 x iSpC3 which is joined to another nucleic acid
aaatctacattgtatgccaggt

SEQ ID NO: 169 - DNA, 22 bp, Artificial Sequence
  Synthetic construct
  misc_feature (1)..(1): cytosine attached to 2 x iSpC3 which is joined to another nucleic acid
cttccagtcggggatgtttaca

SEQ ID NO: 170 - DNA, 23 bp, Artificial Sequence
  Synthetic construct
  misc_feature (1)..(1): guanine attached to 2 x iSpC3 which is joined to another nucleic acid
gctgagagtgtaggatgtttaca

SEQ ID NO: 171 - DNA, 22 bp, Artificial Sequence
  Synthetic construct
  misc_feature (1)..(1): adenine attached to 2 x iSpC3 which is joined to another nucleic acid
atttggttccattttaccagct

SEQ ID NO: 172 - DNA, 22 bp, Artificial Sequence
  Synthetic construct
  misc_feature (1)..(1): guanine attached to 2 x iSpC3 which is joined to another nucleic acid
gtataacccgggccaaaagctc

SEQ ID NO: 173 - DNA, 23 bp, Artificial Sequence
  Synthetic construct
  misc_feature (1)..(1): adenine attached to 2 x iSpC3 which is joined to another nucleic acid
acccaccgacagcaatgaatgtt

SEQ ID NO: 174 - DNA, 22 bp, Artificial Sequence
  Synthetic construct
  misc_feature (1)..(1): thymine attached to 2 x iSpC3 which is joined to another nucleic acid
taaccgatttcagatggtgcta

SEQ ID NO: 175 - DNA, 72 bp, Artificial Sequence
  Synthetic construct
  misc_feature (1)..(1): adenine attached to spacer which is joined to another nucleic acid
atccgtcacacctgctctaggggatcaaagctatgcgaccatgcgagtggatactggtgttggctcccgtat

SEQ ID NO: 176 - DNA, 22 bp, Artificial Sequence
  Synthetic construct
  misc_feature (1)..(1): guanine attached to 2 x iSpC3 which is joined to another nucleic acid
gttcaccaatcagctaagctct

SEQ ID NO: 177 - DNA, 22 bp, Artificial Sequence
  Synthetic construct
  misc_feature (1)..(1): thymine attached to 2 x iSpC3 which is joined to another nucleic acid
tcaacatcagtctgataagcta

SEQ ID NO: 178 - DNA, 22 bp, Artificial Sequence
  Synthetic construct
  misc_feature (1)..(1): adenine attached to 2 x iSpC3 which is joined to another nucleic acid
aaatctacattgtatgccaggt

SEQ ID NO: 179 - DNA, 22 bp, Artificial Sequence
  Synthetic construct
  misc_feature (1)..(1): cytosine attached to 2 x iSpC3 which is joined to another nucleic acid
cttccagtcggggatgtttaca

SEQ ID NO: 180 - DNA, 23 bp, Artificial Sequence
  Synthetic construct
  misc_feature (1)..(1): guanine attached to 2 x iSpC3 which is joined to another nucleic acid
gctgagagtgtaggatgtttaca

SEQ ID NO: 181 - DNA, 23 bp, Artificial Sequence
  Synthetic construct
  misc_feature (1)..(1): adenine attached to 2 x iSpC3 which is joined to another nucleic acid
acccaccgacagcaatgaatgtt

SEQ ID NO: 182 - DNA, 22 bp, Artificial Sequence
  Synthetic construct
  misc_feature (1)..(1): thymine attached to 2 x iSpC3 which is joined to another nucleic acid
taaccgatttcagatggtgcta

SEQ ID NO: 183 - DNA, 22 bp, Artificial Sequence
  Synthetic construct
  misc_feature (1)..(1): cytosine attached to 2 x iSpC3 which is joined to another nucleic acid
cagtgtgcggtgggcaggggct

SEQ ID NO: 184 - DNA, 21 bp, Artificial Sequence
  Synthetic construct
  misc_feature (1)..(1): cytosine attached to 2 x iSpC3 which is joined to another nucleic acid
cgcgtaccaaaagtaataatg

SEQ ID NO: 185 - DNA, 22 bp, Artificial Sequence
  Synthetic construct
  misc_feature (1)..(1): thymine attached to 2 x iSpC3 which is joined to another nucleic acid
tggacgtttgcaggggaggtgg

SEQ ID NO: 186 - DNA, 21 bp, Artificial Sequence
  Synthetic construct
  misc_feature (1)..(1): cytosine attached to 2 x iSpC3 which is joined to another nucleic acid
cgcgtaccaaaagtaataatg

SEQ ID NO: 187 - DNA, 24 bp, Artificial Sequence
  Synthetic construct
  misc_feature (1)..(1): adenine attached to 2 x iSpC3 which is joined to another nucleic acid
actgcaggctccagcttccaggct

SEQ ID NO: 188 - DNA, 22 bp, Artificial Sequence
  Synthetic construct
  misc_feature (1)..(1): cytosine attached to 2 x iSp3C which is joined to another nucleic acid
cttccagtcaaggatgtttaca

SEQ ID NO: 189 - DNA, 23 bp, Artificial Sequence
  Synthetic construct
  misc_feature (1)..(1): cytosine attached to 2 x iSpC3 which is joined to another nucleic acid
ctacctgcactgtaagcactttt

SEQ ID NO: 190 - DNA, 22 bp, Artificial Sequence
  Synthetic construct
  misc_feature (1)..(1): thymine attached to 2 x iSpC3 which is joined to another nucleic acid
taaccaatgtgcagactactgt

SEQ ID NO: 191 - DNA, 21 bp, Artificial Sequence
  Synthetic construct
  misc_feature (1)..(1): cytosine attached to 2 x iSpC3 which is joined to another nucleic acid
cacaaccctagtggcgccatt

SEQ ID NO: 192 - DNA, 21 bp, Artificial Sequence
  Synthetic construct
  misc_feature (1)..(1): adenine attached to 2 x iSpC3 which is joined to another nucleic acid
acctatcctgaattacttgaa

SEQ ID NO: 193 - DNA, 23 bp, Artificial Sequence
  Synthetic construct
  misc_feature (1)..(1): adenine attached to 2 x iSpC3 which is joined to another nucleic acid
agggattcctgggaaaactggac

SEQ ID NO: 194 - DNA, 22 bp, Artificial Sequence
  Synthetic construct
  misc_feature (1)..(1): adenine attached to 2 x iSpC3 which is joined to another nucleic acid
acaggccgggacaagtgcaata

SEQ ID NO: 195 - DNA, 22 bp, Artificial Sequence
  Synthetic construct
  misc_feature (1)..(1): adenine attached to 2 x iSpC3 which is joined to another nucleic acid
aacccatggaattcagttctca

SEQ ID NO: 196 - DNA, 23 bp, Artificial Sequence
  Synthetic construct
  misc_feature (1)..(1): adenine attached to 2 x iSpC3 which is joined to another nucleic acid
aaagtctcgctctctgcccctca

SEQ ID NO: 197 - DNA, 21 bp, Artificial Sequence
  Synthetic construct
  misc_feature (1)..(1): guanine attached to 2 x iSpC3 which is joined to another nucleic acid
gcagaacttagccactgtgaa

SEQ ID NO: 198 - DNA, 22 bp, Artificial Sequence
  Synthetic construct
  misc_feature (1)..(1): adenine attached to 2 x iSpC3 which is joined to another nucleic acid
atacatacttctttacattcca

SEQ ID NO: 199 - DNA, 23 bp, Artificial Sequence
  Synthetic construct
  misc_feature (1)..(1): cytosine attached to 2 x iSpC3 which is joined to another nucleic acid
ctatctgcactagatgcacctta

SEQ ID NO: 200 - DNA, 23 bp, Artificial Sequence
  Synthetic construct
  misc_feature (1)..(1): cytosine attached to 2 x iSpC3 which is joined to another nucleic acid
ctaactgcactagatgcacctta

SEQ ID NO: 201 - DNA, 22 bp, Artificial Sequence
  Synthetic construct
  misc_feature (1)..(1): adenine attached to 2 x iSpC3 which is joined to another nucleic acid
agtagtgcaataaagtcagagc

SEQ ID NO: 202 - DNA, 22 bp, Artificial Sequence
  Synthetic construct
  misc_feature (1)..(1): adenine attached to 2 x iSpC3 which is joined to another nucleic acid
aaccatacaacctactacctca

SEQ ID NO: 203 - DNA, 24 bp, Artificial Sequence
  Synthetic construct
  misc_feature (1)..(1): thymine attached to 2 x iSpC3 which is joined to another nucleic acid
tcacaggttaaagggtctcaggga

SEQ ID NO: 204 - DNA, 21 bp, Artificial Sequence
  Synthetic construct
  misc_feature (1)..(1): adenine attached to 2 x iSpC3 which is joined to another nucleic acid
aggaatatgtttgatatatag

SEQ ID NO: 205 - DNA, 22 bp, Artificial Sequence
  Synthetic construct
  misc_feature (1)..(1): adenine attached to 2 x iSpC3 which is joined to another nucleic acid
agcgggactttgagggccagtt

SEQ ID NO: 206 - DNA, 22 bp, Artificial Sequence
  Synthetic construct
  misc_feature (1)..(1): thymine attached to 2 x iSpC3 which is joined to another nucleic acid
tcatctcgcccgcaaagaccca

SEQ ID NO: 207 - DNA, 22 bp, Artificial Sequence
  Synthetic construct
  misc_feature (1)..(1): adenine attached to 2 x iSpC3 which is joined to another nucleic acid
aggcgaaggatgacaaagggaa

SEQ ID NO: 208 - DNA, 22 bp, Artificial Sequence
  Synthetic construct
  misc_feature (1)..(1): thymine attached to 2 x iSpC3 which is joined to another nucleic acid
tcatctaataaacatttactga

SEQ ID NO: 209 - DNA, 23 bp, Artificial Sequence
  Synthetic construct
  misc_feature (1)..(1): guanine attached to 2 x iSpC3 which is joined to another nucleic acid
gggctcttactccctcaggcact

SEQ ID NO: 210 - DNA, 25 bp, Artificial Sequence
  Synthetic construct
  misc_feature (1)..(1): adenine attached to 2 x iSpC3 which is joined to another nucleic acid
aggccgccacccgcccgcgatccct

SEQ ID NO: 211 - DNA, 23 bp, Artificial Sequence
  Synthetic construct
  misc_feature (1)..(1): cytosine attached to 2 x iSpC3 which is joined to another nucleic acid
ctccagcccctccagggcttcct

SEQ ID NO: 212 - DNA, 22 bp, Artificial Sequence
  Synthetic construct
  misc_feature (1)..(1): thymine attached to 2 x iSpC3 which is joined to another nucleic acid
tgccgtgccctggcctcccact

SEQ ID NO: 213 - DNA, 22 bp, Artificial Sequence
  Synthetic construct
  misc_feature (1)..(1): cytosine attached to 2 x iSpC3 which is joined to another nucleic acid
caccactgcactcgctccagcc

SEQ ID NO: 214 - DNA, 22 bp, Artificial Sequence
  Synthetic construct
  misc_feature (1)..(1): adenine attached to 2 x iSpC3 which is joined to another nucleic acid
aaacagtctacctacattgctc

SEQ ID NO: 215 - DNA, 21 bp, Artificial Sequence
  Synthetic construct
  misc_feature (1)..(1): guanine attached to 2 x iSpC3 which is joined to another nucleic acid
gggaggctcctttgagaattg

SEQ ID NO: 216 - DNA, 76 bp, Artificial Sequence; identical to SEQ ID NO: 49
  Synthetic construct
  misc_feature (1)..(1): adenine attached to 2 x iSpC3 which is joined to another nucleic acid

SEQ ID NO: 217 - DNA, 71 bp, Artificial Sequence
  Synthetic construct
  misc_feature (1)..(1): adenine attached to 2 x iSpC3 which is joined to another nucleic acid
atacgggagccaacaccaggactaacattataagaattgcgaataatcattggagagcaggtgtgacggat

SEQ ID NO: 218 - DNA, 40 bp, Artificial Sequence
  Synthetic construct
  misc_feature (1)..(1): guanine attached to 2 x iSpC3 which is joined to another nucleic acid
ggcgattcgtgatctctgctctcggtttcgcgttcgttcg

SEQ ID NO: 219 - DNA, 29 bp, Artificial Sequence
  Synthetic construct
  misc_feature (1)..(1): adenine attached to 2 x iSpC3 which is joined to another nucleic acid
agtccgtggtagggcaggttggggtgact

SEQ ID NO: 220 - DNA, 15 bp, Artificial Sequence
  Synthetic construct
  misc_feature (1)..(1): guanine attached to 2 x iSpC3 which is joined to another nucleic acid
ggttggtgtggttgg

SEQ ID NO: 221 - DNA, 56 bp, Artificial Sequence
  Synthetic construct
  misc_feature (1)..(1): cytosine attached to 2 x iSpC3 which is joined to another nucleic acid
cacgcataacgtcttgcggggcggcgggttgagaggatgtggggtggttatgcgtg

SEQ ID NO: 222 - DNA, 58 bp, Artificial Sequence
  Synthetic construct
  misc_feature (1)..(1): guanine attached to 2 x iSpC3 which is joined to another nucleic acid
gctggatgtcaccagattgtcggacatcggattgtctgagtcatatgacacatccagc

SEQ ID NO: 223 - DNA, 35 bp, Artificial Sequence; identical to SEQ ID NO: 162
  Synthetic construct
  misc_feature (1)..(1): cytosine attached to 2 x iSpC3 which is joined to another nucleic acid

SEQ ID NO: 224 - DNA, 72 bp, Artificial Sequence; identical to SEQ ID NO: 175
  Synthetic construct
  misc_feature (1)..(1): adenine attached to 2 x iSpC3 which is joined to another nucleic acid

SEQ ID NO: 225 - DNA, 35 bp, Artificial Sequence; identical to SEQ ID NO: 162
  Synthetic construct
  misc_feature (1)..(1): cytosine attached to 2 x iSpC3 which is joined to another nucleic acid

SEQ ID NO: 226 - DNA, 72 bp, Artificial Sequence; identical to SEQ ID NO: 175
  Synthetic construct
  misc_feature (1)..(1): adenine attached to 2 x iSpC3 which is joined to another nucleic acid

SEQ ID NO: 227 - DNA, 44 bp, Artificial Sequence
  Synthetic construct
cgactggtagggagataggggaagctgattcgatgcgtgggtcg

SEQ ID NO: 228 - DNA, 39 bp, Artificial Sequence
  Synthetic construct
gggtgctactcttcctcataagcagtccggtgtatcgat

SEQ ID NO: 229 - DNA, 81 bp, Artificial Sequence
  Synthetic construct
atattgtacttccgtttcgaatttacgtattgctcctagttccgctggtactgaacacaagttcgtcgtacaatacagtgt